(19) United States
(12) Patent Application Publication
(10) Pub. No.: US 2026/0365688 A1
(43) Pub. Date: Nov. 19, 2026

(54) STRAINED BICYCLONONENES

(71) Applicant: TAGWORKS PHARMACEUTICALS B.V., Nijmegen (NL)

(72) Inventors: Bing LIU, Nijmegen (NL); Wolter TEN HOEVE, Assen (NL); Raffaella ROSSIN, Eindhoven (NL); Marc Stefan ROBILLARD, Eindhoven (NL); Laurens Henri Johan KLEIJN, Pittsburgh, PA (US); Ronny Mathieu VERSTEEGEN, Hegelsom (NL)

(73) Assignee: TAGWORKS PHARMACEUTICALS B.V., Nijmegen (NL)

(21) Appl. No.: 19/120,580

(22) PCT Filed: Oct. 12, 2023
(86) PCT No.: PCT/NL2023/050532

§ 371 Date: Apr. 11, 2025

(30) Foreign Application Priority Data
Oct. 12, 2022 (EP) 22201078.7

(51) Int. Cl. C07H 15/26; C07C 62/32; C07C 69/757; C07D 207/46

(52) U.S. Cl. CPC C07H 15/26; C07C 62/32; C07C 69/757; C07D 207/46; C07B 2200/07

(57) ABSTRACT

Disclosed herein are dienophiles, in particular strained bicyclononenes, that can be used in fast bioorthogonal reactions, e.g. with dienes such as tetrazines. The strained bicyclononenes of the disclosure are stable under various conditions, including in vivo environments, and be readily synthesized in fewer steps than required for other dienophiles. Thus, the strained bicyclononenes of the disclosure can be applied in a wide range of applications, particularly in relation to therapeutic applications.

### TECHNICAL FIELD

Disclosed herein are compounds comprising an (E)-bicyclo[6.1.0]non-3-ene moiety, and methods for using and preparing same.

### BACKGROUND

The reactions between dienophiles and dienes are widely used, as these are bioorthogonal and typically very fast. Additionally, a payload can be released from the dienophile, diene, or both upon the reaction between these two compounds. Meanwhile, several other functional moieties can be linked to the dienophile and/or diene as well. These properties render the dienophile-diene reactions interesting for various applications, including those where a drug needs to be released at a specific target site in a subject.

In this light, various dienophiles have been described in literature, including ones where a ring has been fused to a trans-cyclooctene ring so as to further increase the ring strain and thus the reactivity of the dienophile. However, the disadvantages of these molecules are typically that they are also more prone to side-reactions due to the higher reactivity, and/or can only be synthesized using long and labour-intensive routes.

Thus, there is still a need for dienophiles that can be readily synthesized, quickly react with dienes, achieve higher release yields and/or faster release upon reacting with dienes, and/or are more stable in vitro and/or in vivo.

It is desired that compounds be developed that address one or more of the abovementioned problems and/or desires.

### SUMMARY

The disclosure provides the following preferred embodiments, which can be combined with any other embodiment as disclosed herein.

In one aspect, the disclosure relates to a compound, or a salt, solvate, hydrate, and/or an enantiomer thereof, wherein the compound comprises an (E)-bicyclo[6.1.0]non-3-ene moiety, wherein at least one allylic carbon of said moiety is in the R-configuration and is substituted with R₄₈;
R₄₈ is selected from the group consisting of —OH, —O-acetyl, —O—C₁₋₄ alkyl, halogen, active carbonate, and a releasable group;
   the carbon atom at position 1 of said moiety is in the R-configuration;
   the carbon atom at position 8 of said moiety is in the S-configuration; and
preferably the carbon atom at position 9 of said moiety is substituted.

In another aspect, the disclosure relates to a composition comprising:
(a) a compound according to the disclosure, or the salt, solvate, or hydrate thereof, and
(b) the enantiomer of said compound, or the salt, solvate, or hydrate thereof, and
   preferably said composition is a racemic mixture of (a) and (b).

In a further aspect, the disclosure pertains to a combination of
(A1) a compound according to the disclosure, or the salt, solvate, hydrate, and/or enantiomer thereof, or
(A2) a composition according to the disclosure: with
(B) a diene or a salt, solvate, or hydrate thereof; preferably the diene is a tetrazine.

In another aspect, the disclosure relates to a compound according to the disclosure, or the salt, solvate, hydrate, and/or enantiomer thereof, the composition according to the disclosure; or the combination according to the disclosure; for use as a medicament.

In yet another aspect, the disclosure pertains to a compound according to the disclosure, or the salt, solvate, hydrate, and/or enantiomer thereof, the composition according to the disclosure; or the combination according to the disclosure; for use in the treatment of a disease in a subject, preferably the subject is a human, preferably the disease is cancer.

In a further aspect, the disclosure relates to a non-therapeutic method for reacting:
(ia) a compound according to the disclosure, or a salt, solvate, hydrate, and/or an enantiomer thereof, or
(iia) a composition according to the disclosure;
   with a diene or a salt, solvate, or hydrate thereof, wherein said method comprises the step of contacting (ia) or (iia) with said diene or salt, solvate, or hydrate thereof, preferably said contacting is in vitro; and preferably said diene is a tetrazine.

In another aspect, the disclosure relates to an intermediate, or a salt, solvate, hydrate, and/or an enantiomer thereof, selected from the group consisting of Formulae (INT15-1), (INT15-2), (INT15-3), (INT13-1), (INTT13-2), (INTT13-3), (INTAX13-4), and (INTEQ13-4):

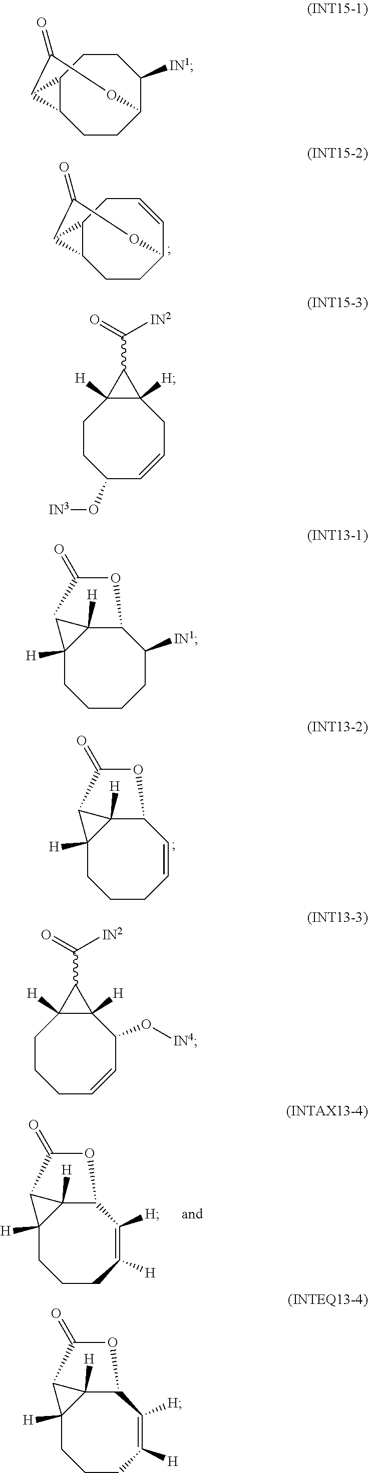

wherein IN¹ is a halogen; IN² is —O—C₁₋₄ alkyl or —OH; IN³ is hydrogen, C₁₋₄ alkyl, or acetyl; IN⁴ is hydrogen, C₁₋₄ alkyl, or acetyl; preferably IN¹ is iodine; preferably IN² is —O—CH₃; preferably IN³ is hydrogen; and preferably IN⁴ is acetyl.

In a further aspect, the disclosure pertains to a method for synthesizing
(i) a compound according to the disclosure, or a salt, solvate, hydrate, and/or an enantiomer thereof, or
(ii) a composition according to the disclosure;
   wherein said method comprises the step of subjecting a compound Z or a salt, solvate, hydrate, and/or an enantiomer thereof, to photoisomerization, wherein compound Z comprises a (Z)-bicyclo[6.1.0]non-3-ene moiety, wherein at least one allylic carbon of said moiety is in the R-configuration and is substituted with R₄₈; R₄₈ is selected from the group consisting of —OH, —O-acetyl, —O—C₁₋₄ alkyl, halogen, active carbonate, and a releasable group; the carbon atom at position 1 of said moiety is in the R-configuration; the carbon atom at position 8 of said moiety is in the S-configuration; and preferably the carbon atom at position 9 of said moiety is substituted; and preferably compound Z is an intermediate, or a salt, solvate, hydrate, and/or an enantiomer thereof; wherein the intermediate is according to Formula (INT15-3) or (INT13-3) as defined herein.

### BRIEF DESCRIPTION OF THE FIGURES

FIG. 1 depicts the results of the kinetc measurements between a compound of the disclosure and a tetrazine as described in Example 4.2. FIG. 1A shows the reaction yields vs. time, and FIG. 1B depicts the pseudo-first-order rate constants vs. the concentration of the compound of the disclosure.

FIG. 2 depicts the % release of N-methylbenzylamine from compound 1.8 according to the disclosure upon reaction with a tetrazine, versus time, as described in Example 4.3.

FIG. 3 relates to the results of Example 4.4. FIG. 3A depicts the In-111/I-125 cpm ratio in the samples obtained from mice plotted vs time. FIG. 3B shows a linear fitting of the data of FIG. 3A, which allowed the calculation of the in vivo half-life of compound of the disclosure 1.8 labeled with iodine-125.

FIG. 4 shows the amino acid sequence of one monomer of diabody AVP0458. AVP0458 consists of two monomers, wherein each of the two monomers has an amino acid sequence according to SEQ ID NO: 1.

### DETAILED DESCRIPTION

The claimed subject-matter, in a broad sense, is based on the judicious insight that the compounds of the disclosure can be readily synthesized in only a few steps, are stable in various conditions, including in physiological environments, react fast in bioorthogonal reactions, and, if required, provide high release yields of a payload connected to an allylic carbon.

Consequently, the claimed subject-matter addresses one or more of the problems and desires as identified herein.

It will be understood that herein, a compound according to claim 1 may be referred to as a dienophile or as “a compound according to the disclosure” or similar phrasing. The (E)-bicyclo[6.1.0]non-3-ene moiety thereof may be referred to as the Trigger or T^{R}. It will be understood that typically the (E)-bicyclo[6.1.0]non-3-ene moiety can be considered a trans-cyclooctene (TCO) moiety fused to a cyclopropyl moiety. Thus, compounds of the disclosure may also be referred to herein as TCO.

Unless stated otherwise, a reference to compounds according to the disclosure is also meant to include all possible salts, solvates, hydrates, and enantiomers thereof.

Moreover, an Activator as referred to herein is a compound that can react with the double bond of the (E)-bicyclo[6.1.0]non-3-ene moiety of the compound of the disclosure. Typically, an Activator is a diene.

### Compounds

The compounds of the disclosure comprise an (E)-bicyclo[6.1.0]non-3-ene moiety. For the sake of clarity, below two examples of such a moiety are drawn wherein the carbon atoms are numbered. These numbers correspond to the positions as mentioned in claim 1. The optional substituents on said moieties are not shown:

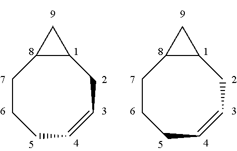

Preferably, in the compounds of the disclosure the vinylic carbons, viz. the carbons at positions 3 and 4, are not substituted. In other words, it is preferred that at the vinylic positions groups —CH═ are present.

Preferably, in the compounds of the disclosure the carbon at position 9 is substituted, more preferably with a group according to RG1 or RG5, more preferably according to RG1. Most preferably, the carbon at position 9 is substituted with a group R₄₉ as defined herein.

In the compounds of the disclosure at least one allylic carbon of the (E)-bicyclo[6.1.0]non-3-ene moiety is in the R-configuration and is substituted with R₄₈. Preferably, only one allylic carbon of the (E)-bicyclo[6.1.0]non-3-ene moiety is in the R-configuration and is substituted with R₄₈
R₄₈ is selected from the group consisting of —OH, —O-acetyl, —O—C₁₋₄ alkyl, halogen, active carbonate, and a releasable group.

In particular when the compound of the disclosure is an intermediate product which may be further adapted, for example to couple said compound to a payload, R₄₈ is preferably selected from the group consisting of —OH, —O-acetyl, —O—C₁₋₄ alkyl, halogen, and an active carbonate.

Active carbonates are well-known to the skilled person. Preferably, the active carbonate is selected from the group consisting of —OC(O)O—N-succinimidyl, —OC(O)O— pentafluorophenyl, —OC(O)O-tetrafluorophenyl, —OC(O)O-4-nitrophenyl, and —OC(O)Cl. More preferably, the active carbonate is —OC(O)O—N-succinimidyl, or —OC(O)O— pentafluorophenyl; most preferably the active carbonate is —OC(O)O—N-succinimidyl.

In particular when the compound of the disclosure is intended to release a payload upon reaction with a diene, it is preferred that R₄₈ is a releasable group. Typically, the releasable group comprises a payload, which is connected to the (E)-bicyclo[6.1.0]non-3-ene moiety in such a way that release occurs upon reaction of the compound of the disclosure with a diene.

While both axial and equatorial isomers are useful and solve the problems indicated above, it is preferred that R₄₈ is in the axial position. Typically, faster reaction rates are observed in bioorthogonal reactions when the compounds of the disclosure have a group R₄₈ in the axial position. Thus, in the Formulae shown below, the ones containing “AX” are preferred over those containing “EQ”, e.g. Formula (II-AX15) is preferred over Formula (II-EQ15).

Likewise, while group R₄₈ can be present on either allylic position (viz. positions 2 and 5 as indicated above), it is preferred that said group be present at the 5 position. Without wishing to be bound by theory, the inventors believe that these molecules are more stable, e.g. in physiological conditions, than the compounds having R₄₈ at the 2 position. Thus, in the Formulae shown below, the ones containing “15” are preferred over those containing “13”, e.g. Formula (II-AX15) is preferred over Formula (II-AX13).

Such releasable groups are well-known and have a clear meaning in the art.

In preferred embodiments, the releasable group is —(Y¹—C(═Y²))ᵢ—(S^{P})ⱼ—C^{A}. Therein, each of Y¹ and Y² are independently selected from O, and S; preferably Y¹ and Y² are O. C^{A} is Construct A, which is the payload. Preferably, C^{A} is an organic molecule or an inorganic molecule. Further preferred embodiments of C^{A} are defined below. For the releasable group, j is 0 or 1; preferably j is 0; and i is 0 or 1; preferably i is 1. If i is 0, —(S^{P})ⱼ—C^{A} is connected to the remainder of the compound via O or S, that is part of —(S^{P})ⱼ—C^{A}. On the other hand, if i is 1, —(S^{P})ⱼ—C^{A} is connected to —C(═Y²)— via O, S, a secondary N, or a tertiary N, that is part of —(S^{P})ⱼ—C^{A}. Preferably, if i is 1, —(S^{P})ⱼ—C^{A} is connected to —C(═Y²)— via a secondary N, or a tertiary N, that is part of —(S^{P})ⱼ—C^{A}.

S^{P} is a spacer, of which preferred embodiments are defined below. Preferably, when S^{P} is part of a releasable group, S^{P} is a self-immolative linker, which is herein also referred to as L^{C}. Such self-immolative linkers are well-known in the art, and preferred embodiments of self-immolative linkers are defined below. If the spacer in the releasable group is a self-immolative linker, upon reaction of a compound of the disclosure with a diene, initially a construct -L^{C}-C^{A} is released. Thereafter, the self-immolative linker self-immolates and releases the payload C^{A}.

Preferably, the compound of the disclosure has a structure according to any one of Formulae (Ia) and (Ib):

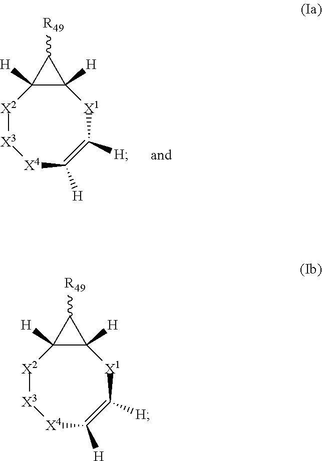

wherein each of X¹, X², X³, and X⁴ is independently a substituted or unsubstituted carbon; at least one of X¹ and X⁴ is —CHR₄₈; preferably one of X¹ and X⁴ is —CHR₄₈; R₄₉ is selected from the group consisting of —C(O)OH, —C(O)O—CH₃, —C(O)NH₂, active esters, and —(S^{P})_{D}—C^{B}; S^{P} is a spacer; D is 0 or 1, preferably D is 1; and C^{B} is a construct B, which is an organic molecule or an inorganic molecule.

The enantiomers of the compounds of Formulae (Ia) and (Ib) are depicted in Formulae (en-Ia) and (en-Ib), respectively:

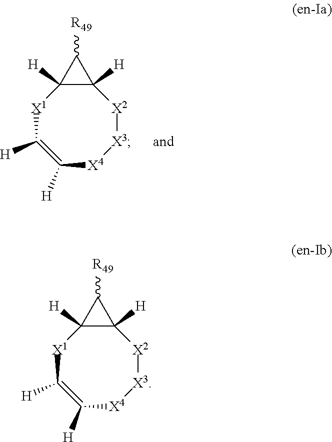

Preferably, if the compound of the disclosure is an intermediate product that may be further adapted, for example to attach a masking moiety or a targeting agent to the compound of the disclosure, R₄₉ is selected from the group consisting of —C(O)OH, —C(O)O—CH₃, —C(O)NH₂, and active esters.

Active esters are well-known to the skilled person. Preferably, for R₄₉ the active ester is selected from the group consisting of —C(O)O—N-succinimidyl, —C(O)O-pentafluorophenyl, —C(O)O-tetrafluorophenyl, —C(O)O-4-nitrophenyl, and —C(O)Cl. More preferably, for R₄₉ the active ester is —C(O)O—N-succinimidyl, or —C(O)O-pentafluorophenyl; most preferably for R₄₉ the active ester is —C(O)O—N-succinimidyl.

Preferably, if the compound of the disclosure is a final product or is intended to release a payload, R₄₉ is —(S^{P})_{D}—C^{B}. This moiety can be used to modulate the pharmacokinetic properties of the compound of the disclosure, further aid in masking the activity of the payload (e.g. if the payload is a drug), and/or target the compound of the disclosure to a certain site, for example in vivo.

In one preferred embodiment, R₄₉ is —(S^{P})_{D}—C^{B}, wherein C^{B} is a polymer, preferably polyethylene glycol. In this embodiment, it is especially preferred that D is 0. As such, R₄₉ modulates the pharmacokinetic properties of the compound of the disclosure, and/or aids in the masking of the payload.

In another preferred embodiment, R₄₉ is —(S^{P})_{D}—C^{B}, wherein C^{B} is a Targeting Agent, preferably an antibody or a diabody, more preferably CC49 or AVP0458. In this embodiment R₄₉ targets the compound of the disclosure. In this embodiment, if D is 1, it is preferred that S^{P} is a polymer, preferably polyethylene glycol. For ease of synthesis, however, D may also be 0 in said embodiment.

Further preferred embodiments of S^{P} and C^{B} are defined below.

In any circumstance, the group —C(O)OH can be advantageously used as R₄₉ to increase the aqueous solubility of compounds of the disclosure, although suitable groups —(S^{P})_{D}—C^{B} can also be selected for R₄₉ in this respect, e.g. if S^{P} and/or C^{B} is a polyethylene glycol or if C^{B} is an amino acid such as glycine.

Preferably, the compound of the disclosure has a structure according to any one of Formulae (II-EQ15), (II-AX15), (II-EQ13), and (II-AX13):

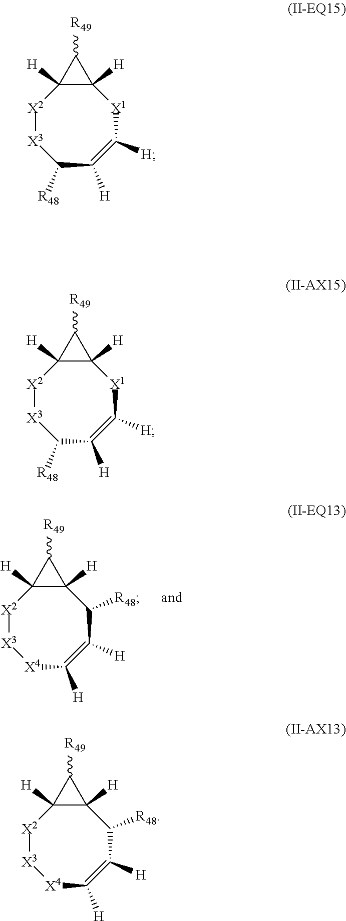

The enantiomers of the compounds of Formulae (II-EQ15), (II-AX15), (II-EQ13), and (II-AX13) are shown in Formulae (en-II-EQ15), (en-II-AX15), (en-II-EQ13), and (en-II-AX13), respectively:

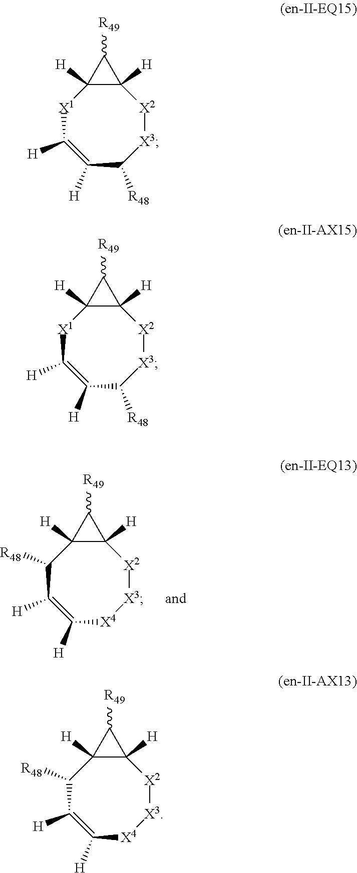

More preferably, the compound of the disclosure has a structure according to any one of Formulae (III-EQ15), (III-AX15), (III-EQ13), and (III-AX13):

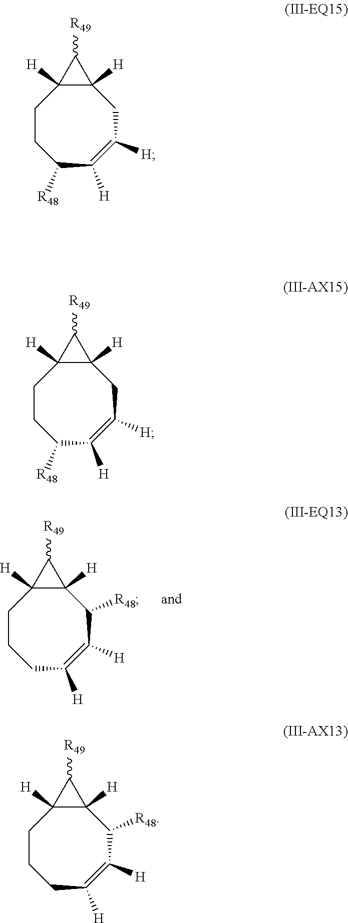

The enantiomers of the compounds of Formulae (III-EQ15), (III-AX15), (III-EQ13), and (III-AX13) are shown in Formulae (en-III-EQ15), (en-III-AX15), (en-III-EQ13), and (en-II-AX13), respectively:

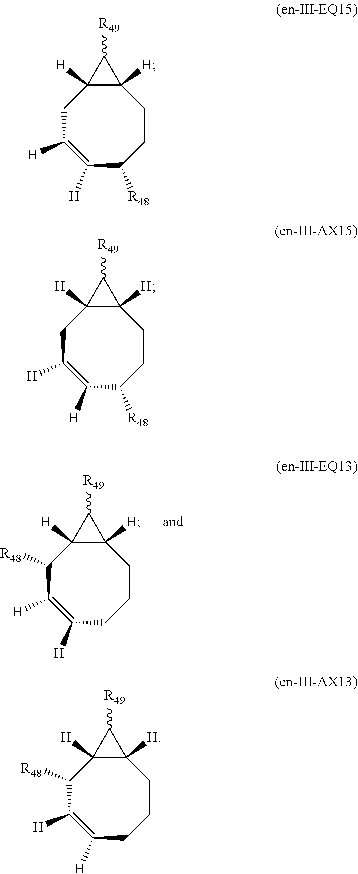

In principle, the stereochemistry of the carbon at position 9, viz. the carbon substituted with R₄₉ in the Formulae depicting compounds of the disclosure, is not relevant. Both endo and exo isomers can be easily obtained, as shown, inter alia, in Examples 1.21 and 1.23 of the present disclosure.

As such, in preferred embodiments the compound according to the disclosure has a structure according to any one of Formulae (IV-EQ15EN), (IV-AX15EN), (IV-EQ13EN), (IV-AX13EN), (IV-EQ15EX), (IV-AX15EX), (IV-EQ13EX), and (IV-AX13EX):

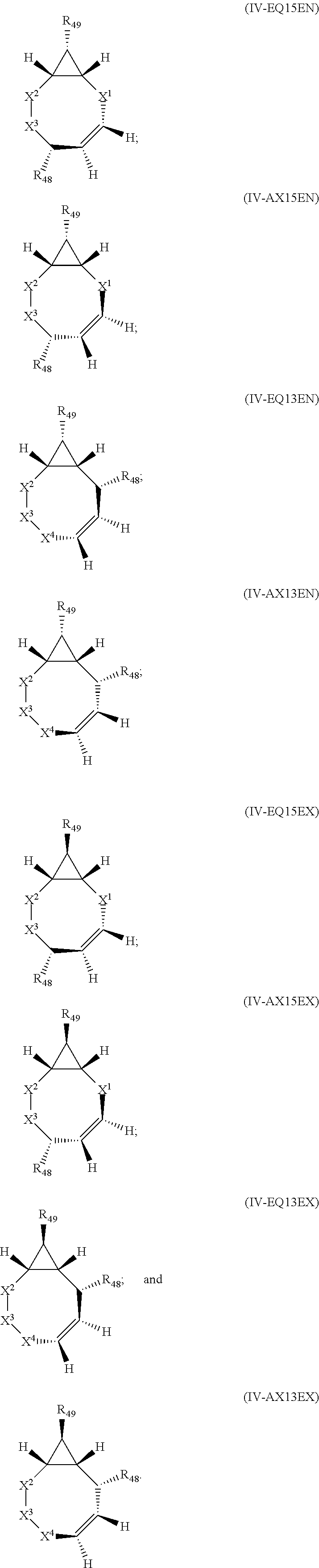

The enantiomers of the compounds of Formulae (IV-EQ15EN), (IV-AX15EN), (IV-EQ13EN), (IV-AX13EN), (IV-EQ15EX), (IV-AX15EX), (IV-EQ13EX), and (IV-AX13EX) are shown in Formulae (en-IV-EQ15EN), (en-IV-AX15EN), (en-IV-EQ13EN), (en-IV-AX13EN), (en-IV-EQ15EX), (en-IV-AX15EX), (en-IV-EQ13EX), and (en-IV-AX13EX), respectively:

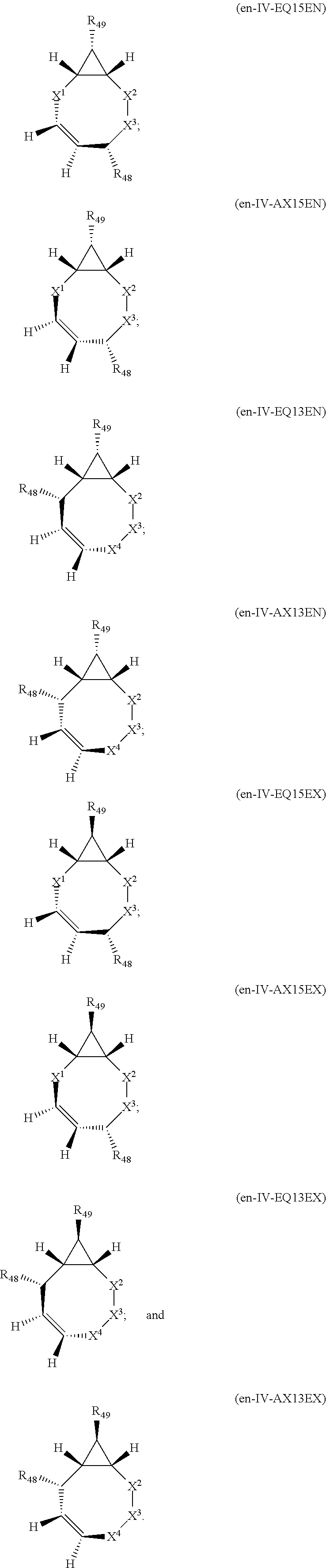

Preferably, the compound according to the disclosure has a structure according to any one of Formulae (V-EQ15EN), (V-AX15EN), (V-EQ13EN), (V-AX13EN), (V-EQ15EX), (V-AX15EX), (V-EQ13EX), and (V-AX13EX):

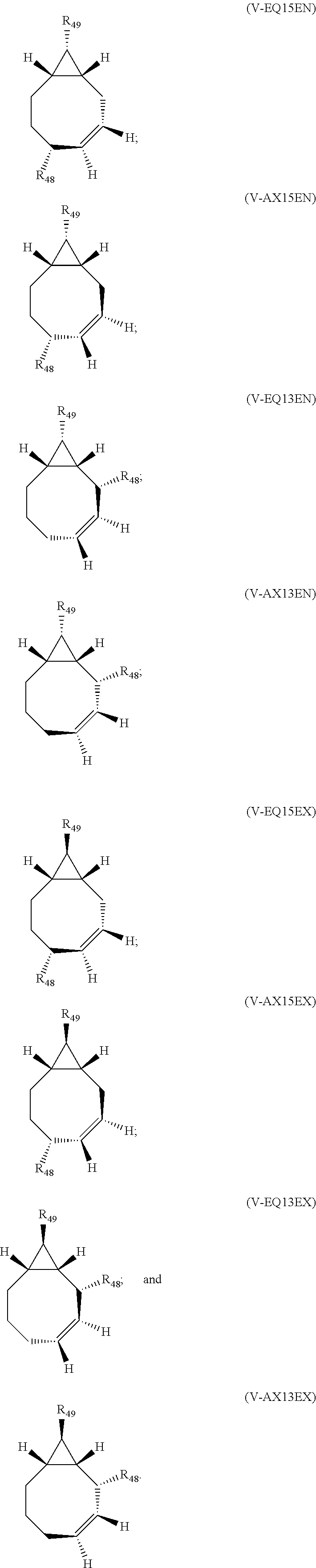

The enantiomers of the compounds of Formulae (V-EQ15EN), (V-AX15EN), (V-EQ13EN), (V-AX13EN), (V-EQ15EX), (V-AX15EX), (V-EQ13EX), and (V-AX13EX) are shown in Formulae (en-V-EQ15EN), (en-V-AX15EN), (en-V-EQ13EN), (en-V-AX13EN), (en-V-EQ15EX), (en-V-AX15EX), (en-V-EQ13EX), and (en-V-AX13EX), respectively:

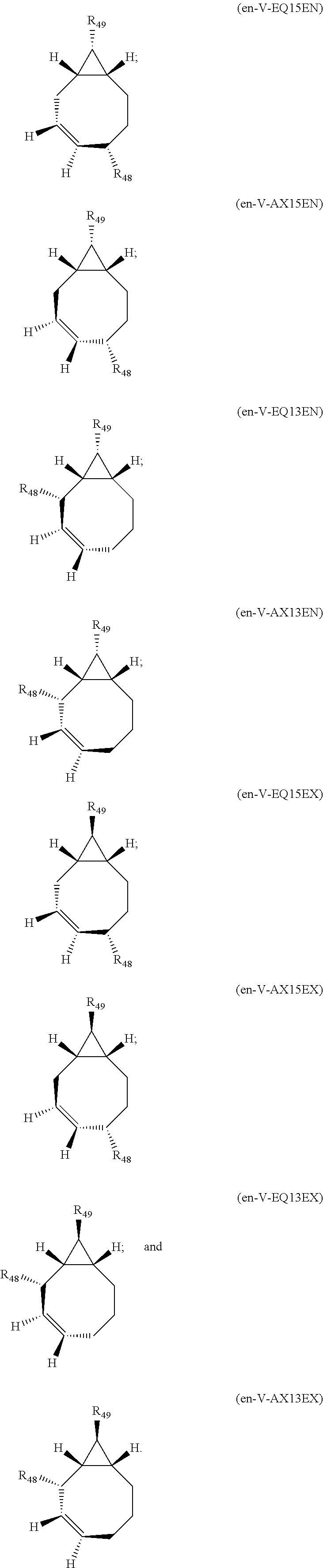

Preferably, in the Formulae depicting compounds of the disclosure comprising X¹, X², X³, and X⁴, each of X¹, X², X³, and X⁴ is independently C(R₄₇)₂; provided that in Formulae (Ia) and (Ib) at least one of X¹ and X⁴ is —CHR₄₈. In this preferred embodiment, for X¹, X², X³, and X⁴ each R₄₇ is independently selected from the group consisting of hydrogen, halogen, (hetero)(cyclo)alkyl, (hetero)(cyclo)alkenyl, (hetero)(cyclo)alkynyl, (hetero)aryl, —(S^{P})_{D}—C^{B}, and combinations thereof; wherein the (hetero)(cyclo)alkyl, (hetero)(cyclo)alkenyl, (hetero)(cyclo)alkynyl, and (hetero)aryl groups are optionally substituted, preferably with a group according to RG1. Preferably, in relation to X¹, X², X³, and X⁴ at most two R₄₇ are not hydrogen, more preferably at most one R₄₇ is not hydrogen; and most preferably all R₄₇ are hydrogen.

Preferably, for a compound according to the disclosure:
(a) R₄₈ is —OH and R₄₉ is —COOH;
(b) R₄₈ is an active carbonate and R₄₉ is an active ester;
(c) R₄₈ is a releasable group and R₄₉ is an active ester; or
(d) R₄₈ is a releasable group and R₄₉ is —(S^{P})_{D}—C^{B}.

Preferably, the compound of the disclosure is according to Formula (Ia) and R₄₈ is —OH and R₄₉ is —COOH. Preferably, the compound of the disclosure is according to Formula (Ia) and R₄₈ is an active carbonate and R₄₉ is an active ester. Preferably, the compound of the disclosure is according to Formula (Ia) and R₄₈ is a releasable group and R₄₉ is an active ester. Preferably, the compound of the disclosure is according to Formula (Ia) and R₄₈ is a releasable group and R₄₉ is —(S^{P})_{D}—C^{B}. Preferably, the compound of the disclosure is according to Formula (Ib) and R₄₈ is —OH and R₄₉ is —COOH. Preferably, the compound of the disclosure is according to Formula (Ib) and R₄₈ is an active carbonate and R₄₉ is an active ester. Preferably, the compound of the disclosure is according to Formula (Ib) and R₄₈ is a releasable group and R₄₉ is an active ester. Preferably, the compound of the disclosure is according to Formula (Ib) and R₄₈ is a releasable group and R₄₉ is —(S^{P})_{D}—C^{B}. Preferably, the compound of the disclosure is according to Formula (II-EQ15) and R₄₈ is —OH and R₄₉ is —COOH. Preferably, the compound of the disclosure is according to Formula (II-EQ15) and R₄₈ is an active carbonate and R₄₉ is an active ester. Preferably, the compound of the disclosure is according to Formula (II-EQ15) and R₄₈ is a releasable group and R₄₉ is an active ester. Preferably, the compound of the disclosure is according to Formula (II-EQ15) and R₄₈ is a releasable group and R₄₉ is —(S^{P})_{D}—C^{B}. Preferably, the compound of the disclosure is according to Formula (II-AX15) and R₄₈ is —OH and R₄₉ is —COOH. Preferably, the compound of the disclosure is according to Formula (II-AX15) and R₄₈ is an active carbonate and R₄₉ is an active ester. Preferably, the compound of the disclosure is according to Formula (II-AX15) and R₄₈ is a releasable group and R₄₉ is an active ester. Preferably, the compound of the disclosure is according to Formula (II-AX15) and R₄₈ is a releasable group and R₄₉ is —(S^{P})_{D}—C^{B}. Preferably, the compound of the disclosure is according to Formula (II-EQ13) and R₄₈ is —OH and R₄₉ is —COOH. Preferably, the compound of the disclosure is according to Formula (II-EQ13) and R₄₈ is an active carbonate and R₄₉ is an active ester. Preferably, the compound of the disclosure is according to Formula (II-EQ13) and R₄₈ is a releasable group and R₄₉ is an active ester. Preferably, the compound of the disclosure is according to Formula (II-EQ13) and R₄₈ is a releasable group and R₄₉ is —(S^{P})_{D}—C^{B}. Preferably, the compound of the disclosure is according to Formula (II-AX13) and R₄₈ is —OH and R₄₉ is —COOH. Preferably, the compound of the disclosure is according to Formula (II-AX13) and R₄₈ is an active carbonate and R₄₉ is an active ester. Preferably, the compound of the disclosure is according to Formula (II-AX13) and R₄₈ is a releasable group and R₄₉ is an active ester. Preferably, the compound of the disclosure is according to Formula (II-AX13) and R₄₈ is a releasable group and R₄₉ is —(S^{P})_{D}—C^{B}. Preferably, the compound of the disclosure is according to Formula (III-EQ15) and R₄₈ is —OH and R₄₉ is —COOH. Preferably, the compound of the disclosure is according to Formula (III-EQ15) and R₄₈ is an active carbonate and R₄₉ is an active ester. Preferably, the compound of the disclosure is according to Formula (III-EQ15) and R₄₈ is a releasable group and R₄₉ is an active ester. Preferably, the compound of the disclosure is according to Formula (III-EQ15) and R₄₈ is a releasable group and R₄₉ is —(S^{P})_{D}—C^{B}. Preferably, the compound of the disclosure is according to Formula (III-AX15) and R₄₈ is —OH and R₄₉ is —COOH. Preferably, the compound of the disclosure is according to Formula (III-AX15) and R₄₈ is an active carbonate and R₄₉ is an active ester. Preferably, the compound of the disclosure is according to Formula (III-AX15) and R₄₈ is a releasable group and R₄₉ is an active ester. Preferably, the compound of the disclosure is according to Formula (III-AX15) and R₄₈ is a releasable group and R₄₉ is —(S^{P})_{D}—C^{B}. Preferably, the compound of the disclosure is according to Formula (III-EQ13) and R₄₈ is —OH and R₄₉ is —COOH. Preferably, the compound of the disclosure is according to Formula (III-EQ13) and R₄₈ is an active carbonate and R₄₉ is an active ester. Preferably, the compound of the disclosure is according to Formula (III-EQ13) and R₄₈ is a releasable group and R₄₉ is an active ester. Preferably, the compound of the disclosure is according to Formula (III-EQ13) and R₄₈ is a releasable group and R₄₉ is —(S^{P})_{D}—C^{B}. Preferably, the compound of the disclosure is according to Formula (III-AX13) and R₄₈ is —OH and R₄₉ is —COOH. Preferably, the compound of the disclosure is according to Formula (III-AX13) and R₄₈ is an active carbonate and R₄₉ is an active ester. Preferably, the compound of the disclosure is according to Formula (III-AX13) and R₄₈ is a releasable group and R₄₉ is an active ester. Preferably, the compound of the disclosure is according to Formula (III-AX13) and R₄₈ is a releasable group and R₄₉ is —(S^{P})_{D}—C^{B}. Preferably, the compound of the disclosure is according to Formula (IV-EQ15EN) and R₄₈ is —OH and R₄₉ is —COOH. Preferably, the compound of the disclosure is according to Formula (IV-EQ15EN) and R₄₈ is an active carbonate and R₄₉ is an active ester. Preferably, the compound of the disclosure is according to Formula (IV-EQ15EN) and R₄₈ is a releasable group and R₄₉ is an active ester. Preferably, the compound of the disclosure is according to Formula (IV-EQ15EN) and R₄₈ is a releasable group and R₄₉ is —(S^{P})_{D}—C^{B}. Preferably, the compound of the disclosure is according to Formula (IV-AX15EN) and R₄₈ is —OH and R₄₉ is —COOH. Preferably, the compound of the disclosure is according to Formula (IV-AX15EN) and R₄₈ is an active carbonate and R₄₉ is an active ester. Preferably, the compound of the disclosure is according to Formula (IV-AX15EN) and R₄₈ is a releasable group and R₄₉ is an active ester. Preferably, the compound of the disclosure is according to Formula (IV-AX15EN) and R₄₈ is a releasable group and R₄₉ is —(S^{P})_{D}—C^{B}. Preferably, the compound of the disclosure is according to Formula (IV-EQ13EN) and R₄₈ is —OH and R₄₉ is —COOH. Preferably, the compound of the disclosure is according to Formula (IV-EQ13EN) and R₄₉ is an active carbonate and R₄₉ is an active ester. Preferably, the compound of the disclosure is according to Formula (IV-EQ13EN) and R₄₉ is a releasable group and R₄₉ is an active ester. Preferably, the compound of the disclosure is according to Formula (IV-EQ13EN) and R₄₈ is a releasable group and R₄₉ is —(S^{P})_{D}—C^{B}. Preferably, the compound of the disclosure is according to Formula (IV-AX13EN) and R₄₈ is —OH and R₄₉ is —COOH. Preferably, the compound of the disclosure is according to Formula (IV-AX13EN) and R₄₈ is an active carbonate and R₄₉ is an active ester. Preferably, the compound of the disclosure is according to Formula (IV-AX13EN) and R₄₉ is a releasable group and R₄₉ is an active ester. Preferably, the compound of the disclosure is according to Formula (IV-AX13EN) and R₄₈ is a releasable group and R₄₉ is —(S^{P})_{D}—C^{B}. Preferably, the compound of the disclosure is according to Formula (IV-EQ15EX) and R₄₉ is —OH and R₄₉ is —COOH. Preferably, the compound of the disclosure is according to Formula (IV-EQ15EX) and R₄₈ is an active carbonate and R₄₉ is an active ester. Preferably, the compound of the disclosure is according to Formula (IV-EQ15EX) and R₄₉ is a releasable group and R₄₉ is an active ester. Preferably, the compound of the disclosure is according to Formula (IV-EQ15EX) and R₄₉ is a releasable group and R₄₉ is —(S^{P})_{D}—C^{B}. Preferably, the compound of the disclosure is according to Formula (IV-AX15EX) and R₄₉ is —OH and R₄₉ is —COOH. Preferably, the compound of the disclosure is according to Formula (IV-AX15EX) and R₄₈ is an active carbonate and R₄₉ is an active ester. Preferably, the compound of the disclosure is according to Formula (IV-AX15EX) and R₄₈ is a releasable group and R₄₉ is an active ester. Preferably, the compound of the disclosure is according to Formula (IV-AX15EX) and R₄₈ is a releasable group and R₄₉ is —(S^{P})_{D}—C^{B}. Preferably, the compound of the disclosure is according to Formula (IV-EQ13EX) and R₄₈ is —OH and R₄₉ is —COOH. Preferably, the compound of the disclosure is according to Formula (IV-EQ13EX) and R₄₉ is an active carbonate and R₄₉ is an active ester. Preferably, the compound of the disclosure is according to Formula (IV-EQ13EX) and R₄₈ is a releasable group and R₄₉ is an active ester. Preferably, the compound of the disclosure is according to Formula (IV-EQ13EX) and R₄₉ is a releasable group and R₄₉ is —(S^{P})_{D}—C^{B}. Preferably, the compound of the disclosure is according to Formula (IV-AX13EX) and R₄₈ is —OH and R₄₉ is —COOH. Preferably, the compound of the disclosure is according to Formula (IV-AX13EX) and R₄₈ is an active carbonate and R₄₉ is an active ester. Preferably, the compound of the disclosure is according to Formula (IV-AX13EX) and R₄₉ is a releasable group and R₄₉ is an active ester. Preferably, the compound of the disclosure is according to Formula (IV-AX13EX) and R₄₉ is a releasable group and R₄₉ is —(S^{P})_{D}—C^{B}. Preferably, the compound of the disclosure is according to Formula (V-EQ15EN) and R₄₈ is —OH and R₄₉ is —COOH. Preferably, the compound of the disclosure is according to Formula (V-EQ15EN) and R₄₈ is an active carbonate and R₄₉ is an active ester. Preferably, the compound of the disclosure is according to Formula (V-EQ15EN) and R₄₉ is a releasable group and R₄₉ is an active ester. Preferably, the compound of the disclosure is according to Formula (V-EQ15EN) and R₄₈ is a releasable group and R₄₉ is —(S^{P})_{D}—C^{B}. Preferably, the compound of the disclosure is according to Formula (V-AX15EN) and R₄₉ is —OH and R₄₉ is —COOH. Preferably, the compound of the disclosure is according to Formula (V-AX15EN) and R₄₈ is an active carbonate and R₄₉ is an active ester. Preferably, the compound of the disclosure is according to Formula (V-AX15EN) and R₄₉ is a releasable group and R₄₉ is an active ester. Preferably, the compound of the disclosure is according to Formula (V-AX15EN) and R₄₈ is a releasable group and R₄₉ is —(S^{P})_{D}—C^{B}. Preferably, the compound of the disclosure is according to Formula (V-EQ13EN) and R₄₉ is —OH and R₄₉ is —COOH. Preferably, the compound of the disclosure is according to Formula (V-EQ13EN) and R₄₈ is an active carbonate and R₄₉ is an active ester. Preferably, the compound of the disclosure is according to Formula (V-EQ13EN) and R₄₈ is a releasable group and R₄₉ is an active ester. Preferably, the compound of the disclosure is according to Formula (V-EQ13EN) and R₄₈ is a releasable group and R₄₉ is —(S^{P})_{D}—C^{B}. Preferably, the compound of the disclosure is according to Formula (V-AX13EN) and R₄₈ is —OH and R₄₉ is —COOH. Preferably, the compound of the disclosure is according to Formula (V-AX13EN) and R₄₈ is an active carbonate and R₄₉ is an active ester. Preferably, the compound of the disclosure is according to Formula (V-AX13EN) and R₄₈ is a releasable group and R₄₉ is an active ester. Preferably, the compound of the disclosure is according to Formula (V-AX13EN) and R₄₈ is a releasable group and R₄₉ is —(S^{P})_{D}—C^{B}. Preferably, the compound of the disclosure is according to Formula (V-EQ15EX) and R₄₈ is —OH and R₄₉ is —COOH. Preferably, the compound of the disclosure is according to Formula (V-EQ15EX) and R₄₈ is an active carbonate and R₄₉ is an active ester. Preferably, the compound of the disclosure is according to Formula (V-EQ15EX) and R₄₈ is a releasable group and R₄₉ is an active ester. Preferably, the compound of the disclosure is according to Formula (V-EQ15EX) and R₄₈ is a releasable group and R₄₉ is —(S^{P})_{D}—C^{B}. Preferably, the compound of the disclosure is according to Formula (V-AX15EX) and R₄₉ is —OH and R₄₉ is —COOH. Preferably, the compound of the disclosure is according to Formula (V-AX15EX) and R₄₈ is an active carbonate and R₄₉ is an active ester. Preferably, the compound of the disclosure is according to Formula (V-AX15EX) and R₄₉ is a releasable group and R₄₉ is an active ester. Preferably, the compound of the disclosure is according to Formula (V-AX15EX) and R₄₈ is a releasable group and R₄₉ is —(S^{P})_{D}—C^{B}. Preferably, the compound of the disclosure is according to Formula (V-EQ13EX) and R₄₈ is —OH and R₄₉ is —COOH. Preferably, the compound of the disclosure is according to Formula (V-EQ13EX) and R₄₈ is an active carbonate and R₄₉ is an active ester. Preferably, the compound of the disclosure is according to Formula (V-EQ13EX) and R₄₈ is a releasable group and R₄₉ is an active ester. Preferably, the compound of the disclosure is according to Formula (V-EQ13EX) and R₄₈ is a releasable group and R₄₉ is —(S^{P})_{D}—C^{B}. Preferably, the compound of the disclosure is according to Formula (V-AX13EX) and R₄₈ is —OH and R₄₉ is —COOH. Preferably, the compound of the disclosure is according to Formula (V-AX13EX) and R₄₈ is an active carbonate and R₄₉ is an active ester. Preferably, the compound of the disclosure is according to Formula (V-AX13EX) and R₄₈ is a releasable group and R₄₉ is an active ester. Preferably, the compound of the disclosure is according to Formula (V-AX13EX) and R₄₈ is a releasable group and R₄₉ is —(S^{P})_{D}—C^{B}. Preferably, the compound of the disclosure is according to Formula (en-Ia) and R₄₈ is —OH and R₄₉ is —COOH. Preferably, the compound of the disclosure is according to Formula (en-Ia) and R₄₈ is an active carbonate and R₄₉ is an active ester. Preferably, the compound of the disclosure is according to Formula (en-Ia) and R₄₈ is a releasable group and R₄₉ is an active ester. Preferably, the compound of the disclosure is according to Formula (en-Ia) and R₄₈ is a releasable group and R₄₉ is —(S^{P})_{D}—C^{B}. Preferably, the compound of the disclosure is according to Formula (en-Ib) and R₄₈ is —OH and R₄₉ is —COOH. Preferably, the compound of the disclosure is according to Formula (en-Ib) and R₄₈ is an active carbonate and R₄₉ is an active ester. Preferably, the compound of the disclosure is according to Formula (en-Ib) and R₄₈ is a releasable group and R₄₉ is an active ester. Preferably, the compound of the disclosure is according to Formula (en-Ib) and R₄₈ is a releasable group and R₄₉ is —(S^{P})_{D}—C^{B}. Preferably, the compound of the disclosure is according to Formula (en-II-EQ15) and R₄₈ is —OH and R₄₉ is —COOH. Preferably, the compound of the disclosure is according to Formula (en-II-EQ15) and R₄₈ is an active carbonate and R₄₉ is an active ester. Preferably, the compound of the disclosure is according to Formula (en-II-EQ15) and R₄₈ is a releasable group and R₄₉ is an active ester. Preferably, the compound of the disclosure is according to Formula (en-II-EQ15) and R₄₈ is a releasable group and R₄₉ is —(S^{P})_{D}—C^{B}. Preferably, the compound of the disclosure is according to Formula (en-II-AX15) and R₄₈ is —OH and R₄₉ is —COOH. Preferably, the compound of the disclosure is according to Formula (en-II-AX15) and R₄₈ is an active carbonate and R₄₉ is an active ester. Preferably, the compound of the disclosure is according to Formula (en-II-AX15) and R₄₈ is a releasable group and R₄₉ is an active ester. Preferably, the compound of the disclosure is according to Formula (en-II-AX15) and R₄₈ is a releasable group and R₄₉ is —(S^{P})_{D}—C^{B}. Preferably, the compound of the disclosure is according to Formula (en-II-EQ13) and R₄₈ is —OH and R₄₉ is —COOH. Preferably, the compound of the disclosure is according to Formula (en-II-EQ13) and R₄₈ is an active carbonate and R₄₉ is an active ester. Preferably, the compound of the disclosure is according to Formula (en-II-EQ13) and R₄₈ is a releasable group and R₄₉ is an active ester. Preferably, the compound of the disclosure is according to Formula (en-II-EQ13) and R₄₈ is a releasable group and R₄₉ is —(S^{P})_{D}—C^{B}. Preferably, the compound of the disclosure is according to Formula (en-II-AX13) and R₄₈ is —OH and R₄₉ is —COOH. Preferably, the compound of the disclosure is according to Formula (en-II-AX13) and R₄₈ is an active carbonate and R₄₉ is an active ester. Preferably, the compound of the disclosure is according to Formula (en-II-AX13) and R₄₈ is a releasable group and R₄₉ is an active ester. Preferably, the compound of the disclosure is according to Formula (en-II-AX13) and R₄₈ is a releasable group and R₄₉ is —(S^{P})_{D}—C^{B}. Preferably, the compound of the disclosure is according to Formula (en-III-EQ15) and R₄₈ is —OH and R₄₉ is —COOH. Preferably, the compound of the disclosure is according to Formula (en-III-EQ15) and R₄₈ is an active carbonate and R₄₉ is an active ester. Preferably, the compound of the disclosure is according to Formula (en-III-EQ15) and R₄₈ is a releasable group and R₄₉ is an active ester. Preferably, the compound of the disclosure is according to Formula (en-III-EQ15) and R₄₈ is a releasable group and R₄₉ is —(S^{P})_{D}—C^{B}. Preferably, the compound of the disclosure is according to Formula (en-III-AX15) and R₄₈ is —OH and R₄₉ is —COOH. Preferably, the compound of the disclosure is according to Formula (en-III-AX15) and R₄₈ is an active carbonate and R₄₉ is an active ester. Preferably, the compound of the disclosure is according to Formula (en-III-AX15) and R₄₈ is a releasable group and R₄₉ is an active ester. Preferably, the compound of the disclosure is according to Formula (en-III-AX15) and R₄₈ is a releasable group and R₄₉ is —(S^{P})_{D}—C^{B}. Preferably, the compound of the disclosure is according to Formula (en-III-EQ13) and R₄₈ is —OH and R₄₉ is —COOH. Preferably, the compound of the disclosure is according to Formula (en-III-EQ13) and R₄₈ is an active carbonate and R₄₉ is an active ester. Preferably, the compound of the disclosure is according to Formula (en-III-EQ13) and R₄₈ is a releasable group and R₄₉ is an active ester. Preferably, the compound of the disclosure is according to Formula (en-III-EQ13) and R₄₈ is a releasable group and R₄₉ is —(S^{P})_{D}—C^{B}. Preferably, the compound of the disclosure is according to Formula (en-III-AX13) and R₄₈ is —OH and R₄₉ is —COOH. Preferably, the compound of the disclosure is according to Formula (en-III-AX13) and R₄₈ is an active carbonate and R₄₉ is an active ester. Preferably, the compound of the disclosure is according to Formula (en-III-AX13) and R₄₈ is a releasable group and R₄₉ is an active ester. Preferably, the compound of the disclosure is according to Formula (en-III-AX13) and R₄₉ is a releasable group and R₄₉ is —(S^{P})_{D}—C^{B}. Preferably, the compound of the disclosure is according to Formula (en-IV-EQ15EN) and R₄₈ is —OH and R₄₉ is —COOH. Preferably, the compound of the disclosure is according to Formula (en-IV-EQ15EN) and R₄₈ is an active carbonate and R₄₉ is an active ester. Preferably, the compound of the disclosure is according to Formula (en-IV-EQ15EN) and R₄₈ is a releasable group and R₄₉ is an active ester. Preferably, the compound of the disclosure is according to Formula (en-IV-EQ15EN) and R₄₈ is a releasable group and R₄₉ is —(S^{P})_{D}—C^{B}. Preferably, the compound of the disclosure is according to Formula (en-IV-AX15EN) and R₄₈ is —OH and R₄₉ is —COOH. Preferably, the compound of the disclosure is according to Formula (en-IV-AX15EN) and R₄₈ is an active carbonate and R₄₉ is an active ester. Preferably, the compound of the disclosure is according to Formula (en-IV-AX15EN) and R₄₈ is a releasable group and R₄₉ is an active ester. Preferably, the compound of the disclosure is according to Formula (en-IV-AX15EN) and R₄₈ is a releasable group and R₄₉ is —(S^{P})_{D}—C^{B}. Preferably, the compound of the disclosure is according to Formula (en-IV-EQ13EN) and R₄₈ is —OH and R₄₉ is —COOH. Preferably, the compound of the disclosure is according to Formula (en-IV-EQ13EN) and R₄₈ is an active carbonate and R₄₉ is an active ester. Preferably, the compound of the disclosure is according to Formula (en-IV-EQ13EN) and R₄₈ is a releasable group and R₄₉ is an active ester. Preferably, the compound of the disclosure is according to Formula (en-IV-EQ13EN) and R₄₈ is a releasable group and R₄₉ is —(S^{P})_{D}—C^{B}. Preferably, the compound of the disclosure is according to Formula (en-IV-AX13EN) and R₄₈ is —OH and R₄₉ is —COOH. Preferably, the compound of the disclosure is according to Formula (en-IV-AX13EN) and R₄₈ is an active carbonate and R₄₉ is an active ester. Preferably, the compound of the disclosure is according to Formula (en-IV-AX13EN) and R₄₈ is a releasable group and R₄₉ is an active ester. Preferably, the compound of the disclosure is according to Formula (en-IV-AX13EN) and R₄₈ is a releasable group and R₄₉ is —(S^{P})_{D}—C^{B}. Preferably, the compound of the disclosure is according to Formula (en-IV-EQ15EX) and R₄₈ is —OH and R₄₉ is —COOH. Preferably, the compound of the disclosure is according to Formula (en-IV-EQ15EX) and R₄₈ is an active carbonate and R₄₉ is an active ester. Preferably, the compound of the disclosure is according to Formula (en-IV-EQ15EX) and R₄₈ is a releasable group and R₄₉ is an active ester. Preferably, the compound of the disclosure is according to Formula (en-IV-EQ15EX) and R₄₈ is a releasable group and R₄₉ is —(S^{P})_{D}—C^{B}. Preferably, the compound of the disclosure is according to Formula (en-IV-AX15EX) and R₄₈ is —OH and R₄₉ is —COOH. Preferably, the compound of the disclosure is according to Formula (en-IV-AX15EX) and R₄₈ is an active carbonate and R₄₉ is an active ester. Preferably, the compound of the disclosure is according to Formula (en-IV-AX15EX) and R₄₈ is a releasable group and R₄₉ is an active ester. Preferably, the compound of the disclosure is according to Formula (en-IV-AX15EX) and R₄₈ is a releasable group and R₄₉ is —(S^{P})_{D}—C^{B}. Preferably, the compound of the disclosure is according to Formula (en-IV-EQ13EX) and R₄₈ is —OH and R₄₉ is —COOH. Preferably, the compound of the disclosure is according to Formula (en-IV-EQ13EX) and R₄₈ is an active carbonate and R₄₉ is an active ester. Preferably, the compound of the disclosure is according to Formula (en-IV-EQ13EX) and R₄₈ is a releasable group and R₄₉ is an active ester. Preferably, the compound of the disclosure is according to Formula (en-IV-EQ13EX) and R₄₈ is a releasable group and R₄₉ is —(S^{P})_{D}—C^{B}. Preferably, the compound of the disclosure is according to Formula (en-IV-AX13EX) and R₄₈ is —OH and R₄₉ is —COOH. Preferably, the compound of the disclosure is according to Formula (en-IV-AX13EX) and R₄₈ is an active carbonate and R₄₉ is an active ester. Preferably, the compound of the disclosure is according to Formula (en-IV-AX13EX) and R₄₈ is a releasable group and R₄₉ is an active ester. Preferably, the compound of the disclosure is according to Formula (en-IV-AX13EX) and R₄₈ is a releasable group and R₄₉ is —(S^{P})_{D}—C^{B}. Preferably, the compound of the disclosure is according to Formula (en-V-EQ15EN) and R₄₈ is —OH and R₄₉ is —COOH. Preferably, the compound of the disclosure is according to Formula (en-V-EQ15EN) and R₄₈ is an active carbonate and R₄₉ is an active ester. Preferably, the compound of the disclosure is according to Formula (en-V-EQ15EN) and R₄₈ is a releasable group and R₄₉ is an active ester. Preferably, the compound of the disclosure is according to Formula (en-V-EQ15EN) and R₄₈ is a releasable group and R₄₉ is —(S^{P})_{D}—C^{B}. Preferably, the compound of the disclosure is according to Formula (en-V-AX15EN) and R₄₈ is —OH and R₄₉ is —COOH. Preferably, the compound of the disclosure is according to Formula (en-V-AX15EN) and R₄₈ is an active carbonate and R₄₉ is an active ester. Preferably, the compound of the disclosure is according to Formula (en-V-AX15EN) and R₄₈ is a releasable group and R₄₉ is an active ester. Preferably, the compound of the disclosure is according to Formula (en-V-AX15EN) and R₄₈ is a releasable group and R₄₉ is —(S^{P})_{D}—C^{B}. Preferably, the compound of the disclosure is according to Formula (en-V-EQ13EN) and R₄₈ is —OH and R₄₉ is —COOH. Preferably, the compound of the disclosure is according to Formula (en-V-EQ13EN) and R₄₈ is an active carbonate and R₄₉ is an active ester. Preferably, the compound of the disclosure is according to Formula (en-V-EQ13EN) and R₄₈ is a releasable group and R₄₉ is an active ester. Preferably, the compound of the disclosure is according to Formula (en-V-EQ13EN) and R₄₈ is a releasable group and R₄₉ is —(S^{P})_{D}—C^{B}. Preferably, the compound of the disclosure is according to Formula (en-V-AX13EN) and R₄₉ is —OH and R₄₉ is —COOH. Preferably, the compound of the disclosure is according to Formula (en-V-AX13EN) and R₄₈ is an active carbonate and R₄₉ is an active ester. Preferably, the compound of the disclosure is according to Formula (en-V-AX13EN) and R₄₈ is a releasable group and R₄₉ is an active ester. Preferably, the compound of the disclosure is according to Formula (en-V-AX13EN) and R₄₈ is a releasable group and R₄₉ is —(S^{P})_{D}—C^{B}. Preferably, the compound of the disclosure is according to Formula (en-V-EQ15EX) and R₄₈ is —OH and R₄₉ is —COOH. Preferably, the compound of the disclosure is according to Formula (en-V-EQ15EX) and R₄₈ is an active carbonate and R₄₉ is an active ester. Preferably, the compound of the disclosure is according to Formula (en-V-EQ15EX) and R₄₈ is a releasable group and R₄₉ is an active ester. Preferably, the compound of the disclosure is according to Formula (en-V-EQ15EX) and R₄₈ is a releasable group and R₄₉ is —(S^{P})_{D}—C^{B}. Preferably, the compound of the disclosure is according to Formula (en-V-AX15EX) and R₄₈ is —OH and R₄₉ is —COOH. Preferably, the compound of the disclosure is according to Formula (en-V-AX15EX) and R₄₈ is an active carbonate and R₄₉ is an active ester. Preferably, the compound of the disclosure is according to Formula (en-V-AX15EX) and R₄₈ is a releasable group and R₄₉ is an active ester. Preferably, the compound of the disclosure is according to Formula (en-V-AX15EX) and R₄₈ is a releasable group and R₄₉ is —(S^{P})_{D}—C^{B}. Preferably, the compound of the disclosure is according to Formula (en-V-EQ13EX) and R₄₈ is —OH and R₄₉ is —COOH. Preferably, the compound of the disclosure is according to Formula (en-V-EQ13EX) and R₄₈ is an active carbonate and R₄₉ is an active ester. Preferably, the compound of the disclosure is according to Formula (en-V-EQ13EX) and R₄₈ is a releasable group and R₄₉ is an active ester. Preferably, the compound of the disclosure is according to Formula (en-V-EQ13EX) and R₄₈ is a releasable group and R₄₉ is —(S^{P})_{D}—C^{B}. Preferably, the compound of the disclosure is according to Formula (en-V-AX13EX) and R₄₈ is —OH and R₄₉ is —COOH. Preferably, the compound of the disclosure is according to Formula (en-V-AX13EX) and R₄₈ is an active carbonate and R₄₉ is an active ester. Preferably, the compound of the disclosure is according to Formula (en-V-AX13EX) and R₄₈ is a releasable group and R₄₉ is an active ester. Preferably, the compound of the disclosure is according to Formula (en-V-AX13EX) and R₄₈ is a releasable group and R₄₉ is —(S^{P})_{D}—C^{B}.

### Construct A (C^{A})

C^{A} as described herein is a payload. Importantly, release occurs upon reaction of a compound of the disclosure with a diene, regardless of the nature of C^{A}, with typically the only requirement being that C^{A} be connected to the allylic carbon of the (E)-bicyclo[6.1.0]non-3-ene moiety as defined herein. This is confirmed by various reaction mechanisms for the release, as for example disclosed in WO 2020/256546 (Scheme 2, page 39).

As such, C^{A} is preferably an organic molecule or an inorganic molecule. More preferably, Construct A is according to RG3 or RG4. Even more preferably, C^{A} is selected from the group consisting of a drug, a nucleic acid, a peptide, a protein, a carbohydrate, an aptamer, a hormone, a toxin, a steroid, a cytokine, a lipid, a small organic molecule, a polymer, LNA, PNA, an amino acid, a peptoid, a chelating moiety, a molecule comprising a radionuclide, a fluorescent dye, a phosphorescent dye, a resin, a bead, an organic particle, a gel, an organic surface, an organometallic compound, a cell, an inorganic surface, an inorganic particle, an allotrope of carbon, an inorganic drug, a radionuclide, and combinations thereof. Even more preferably C^{A} is a drug, more preferably still C^{A} is a cancer drug. Most preferably, C^{A} is a protein, a toxin, a chelating moiety, monomethyl auristatin E, or doxorubicin; wherein preferably the chelating moiety comprises a radionuclide.

Further preferred embodiments of the categories from which Construct A can be chosen (i.e. drugs, nucleic acids, petpides, etc.) are further defined below and also apply to Construct A.

### Construct B (C^{B})

Preferably, the dienes and dienophiles as disclosed herein, especially Trigger moieties, also contain one or more, preferably at most two, and most preferably one, construct B (C^{B}) as defined herein. C^{B} may be attached to the remainder of the dienophile or diene via a spacer S′ as defined herein. In general, each C^{B} is independently an organic molecule or an inorganic molecule. C^{B} may have one or more functions, and may have multiple functions at the same time. C^{B} may act as a masking moiety, a targeting agent, and/or a pharmacokinetics-modulating moiety (e.g. a half-life extension moiety). In its latter purpose, C^{B} can for example be used to optimize the aqueous solubility of the dienophile, and/or the diene, for example if C^{B} is an amino acid, in particular glycine.

Preferably, each C^{B} is independently selected from the group consisting of organic molecules, and inorganic molecules. More preferably, Construct B is according to RG3 or RG4. Thus, C^{B} is preferably selected from the group consisting of a nucleic acid, a peptide, a protein, a carbohydrate, an aptamer, a hormone, a toxin, a steroid, a cytokine, a lipid, a small organic molecule as defined herein, a polymer, LNA, PNA, an amino acid, a peptoid, a chelating moiety, a molecule comprising a radionuclide, a fluorescent dye, a phosphorescent dye, a drug, a resin, a bead, an organic particle, a gel, an organic surface, an organometallic compound, a cell, an inorganic surface, an inorganic particle, an allotrope of carbon, an inorganic drug, a radionuclide, and combinations thereof. More preferably C^{B} is a peptide, protein or a polymer, and most preferably C^{B} is an antibody or polyethylene glycol.

If C^{B} is part of the dienophile, C^{B} preferably comprises a polymer, and more preferably C^{B} comprises polyethylene glycol (PEG). This is advantageous, as the polymer typically enables further masking of the payload and/or to optimize the pharmacokinetic properties of the dienophile. Preferably, C^{B} is a PEG moiety coupled to a targeting agent as defined herein, preferably an antibody, optionally via a spacer S′ as defined herein. Preferably, C^{B} comprises or is a PEG moiety of at most 40,000 Da, more preferably at most 30,000 Da, even more preferably at most 25,000 Da, more preferably still at most 20,000 Da, even more preferably at most 15,000 Da, yet more preferably at most 10,000 Da, even more preferably at most 5,000 Da, more preferably at most 2,500 Da, more preferably still at most 1,000 Da, most preferably at most 500 Da. Preferably, C^{B} comprises or is a PEG moiety having of from 1 to 114 repeating units, more preferably of from 2 to 57 repeating units, even more preferably of from 3 to 30 repeating units, and most preferably of from 4 to 20 repeating units. Preferably, C^{B} comprises or is a PEG moiety having at most 24 repeating units, more preferably at most 2 repeating units. The PEG moiety of C^{B} can be linear or branched. Preferably, C^{B} is a polymer, and more preferably C^{B} is polyethylene glycol (PEG).

Preferably, C^{B} is a polymer, preferably PEG, linked, optionally via a spacer S′ as defined herein, to a Targeting Agent, preferably an antibody. In this embodiment, it is preferred that the polymer is closest to the (E)-bicyclo[6.1.0]non-3-ene moiety or the tetrazine moiety, i.e. that the polymer acts as a linker between said moiety and the Targeting Agent.

Preferably, especially if C^{B} acts as a targeting agent, C^{B} is a small molecule, a carbohydrate, biotin, peptide, peptoid, lipid, protein, oligonucleotide, DNA, RNA, PNA, LNA, aptamer, hormone, toxin, steroid, cytokine, antibody, antibody fragment (e.g. Fab2, Fab, scFV, diabody, triabodies, VHH), and antibody (fragment) fusions (e.g. bi-specific and trispecific mAb fragments). In other embodiments C^{B} is a drug or an imaging probe such as a fluorscent dye.

Construct B can also be a radical according to RG1f or a moiety comprising RG1f, as defined herein, wherein RG1f can be used to bind to a further Construct B. For example, Construct B can be RG1f being a maleimide or photocrosslinker that is bound to the remainder of the dienophile (of which the (E)-bicyclo[6.1.0]non-3-ene moiety may be referred to as the Trigger or T^{R}) via a Spacer S^{P}. The maleimide or photocrosslinker can be used to further conjugate the T^{R} to another Construct-B such as a protein. In this particular embodiment C^{B} is a biomolecule-binding moiety.

In another preferred embodiment, each C^{B} is independently a radical according to RG5 as defined herein. Preferably, each C^{B} is independently a masking moiety as defined herein. Preferably, each C^{B} is independently a targeting agent as defined herein. As noted herein, the masking moiety may also act as a targeting agent and vice versa, so that C^{B} can also have multiple functions.

Preferably, at most three C^{B} are comprised in the dienophile, more preferably at most two, most preferably at most one C^{B} is comprised in the dienophile.

When C^{B} is present in the Trigger, Preferably C^{B} is bound to the remainder of the molecule via a residue of RG1f as defined herein, wherein preferably said residue of RG1f equals or is comprised in a Spacer. A person skilled in the art will understand that “residue of RG1f” means the conjugation reaction product of RG1f with another chemical group so as to form a conjugate between C^{B} with the Trigger, L^{C} or S^{P}.

Preferably, when C^{B} is present in the dienophile, C^{B} is bound to the remainder of the molecule via a radical according to any one of RG2a, RG2b, and RG2c as defined herein, wherein preferably RG2a, RG2b, and RG2c equals or is comprised in a Spacer.

### Compositions

The disclosure also pertains to compositions comprising:
(a) a compound according to the disclosure, or the salt, solvate, or hydrate thereof, and
(b) the enantiomer of said compound, or the salt, solvate, or hydrate thereof.

Preferably, in said composition the molar ratio of (a) over (b) is in a range of from 1:4 to 4:1, more preferably in a range of from 1:3 to 3:1, even more preferably in a range of from 1:2 to 2:1, more preferably still in a range of from 1:1.5 to 1.5:1. Most preferably said composition is a racemic mixture of (a) and (b), i.e. the molar ratio of (a) over (b) is about 1:1.

Preferably, (a) is a compound of Formula (Ia) and (b) is a compound of Formula (en-Ia). Preferably, (a) is a compound of Formula (Ib) and (b) is a compound of Formula (en-Ib). Preferably, (a) is a compound of Formula (II-EQ15) and (b) is a compound of Formula (en-II-EQ15). Preferably, (a) is a compound of Formula (II-AX15) and (b) is a compound of Formula (en-II-AX15). Preferably, (a) is a compound of Formula (II-EQ13) and (b) is a compound of Formula (en-II-EQ13). Preferably, (a) is a compound of Formula (II-AX13) and (b) is a compound of Formula (en-II-AX13). Preferably, (a) is a compound of Formula (III-EQ15) and (b) is a compound of Formula (en-III-EQ15). Preferably, (a) is a compound of Formula (III-AX15) and (b) is a compound of Formula (en-III-AX15). Preferably, (a) is a compound of Formula (III-EQ13) and (b) is a compound of Formula (en-III-EQ13). Preferably, (a) is a compound of Formula (III-AX13) and (b) is a compound of Formula (en-III-AX13). Preferably, (a) is a compound of Formula (IV-EQ15EN) and (b) is a compound of Formula (en-IV-EQ15EN). Preferably, (a) is a compound of Formula (IV-AX15EN) and (b) is a compound of Formula (en-IV-AX15EN). Preferably, (a) is a compound of Formula (IV-AX15EN) and (b) is a compound of Formula (en-IV-AX15EN). Preferably, (a) is a compound of Formula (IV-AX15EN) and (b) is a compound of Formula (en-IV-AX15EN). Preferably, (a) is a compound of Formula (IV-EQ13EN) and (b) is a compound of Formula (en-IV-EQ13EN). Preferably, (a) is a compound of Formula (IV-AX13EN) and (b) is a compound of Formula (en-IV-AX13EN). Preferably, (a) is a compound of Formula (IV-EQ15EX) and (b) is a compound of Formula (en-IV-EQ15EX). Preferably, (a) is a compound of Formula (IV-AX15EX) and (b) is a compound of Formula (en-IV-AX15EX). Preferably, (a) is a compound of Formula (IV-EQ13EX) and (b) is a compound of Formula (en-IV-EQ13EX). Preferably, (a) is a compound of Formula (IV-AX13EX) and (b) is a compound of Formula (en-IV-AX13EX). Preferably, (a) is a compound of Formula (V-EQ15EN) and (b) is a compound of Formula (en-V-EQ15EN). Preferably, (a) is a compound of Formula (V-AX15EN) and (b) is a compound of Formula (en-V-AX15EN). Preferably, (a) is a compound of Formula (V-AX15EN) and (b) is a compound of Formula (en-V-AX15EN). Preferably, (a) is a compound of Formula (V-AX15EN) and (b) is a compound of Formula (en-V-AX15EN). Preferably, (a) is a compound of Formula (V-EQ13EN) and (b) is a compound of Formula (en-V-EQ13EN). Preferably, (a) is a compound of Formula (V-AX13EN) and (b) is a compound of Formula (en-V-AX13EN). Preferably, (a) is a compound of Formula (V-EQ15EX) and (b) is a compound of Formula (en-V-EQ15EX). Preferably, (a) is a compound of Formula (V-AX15EX) and (b) is a compound of Formula (en-V-AX15EX). Preferably, (a) is a compound of Formula (V-EQ13EX) and (b) is a compound of Formula (en-V-EQ13EX). Preferably, (a) is a compound of Formula (AX13EX) and (b) is a compound of Formula (en-V-AX13EX).

### INTERMEDIATES

The disclosure also pertains to intermediates, or salts, solvates, hydrates, and/or enantiomers thereof. These intermediates are advantageous, since they result in a short synthetic route towards the compounds of the disclosure. The intermediates are selected from the group consisting of Formulae (INT15-1), (INT15-2), (INT15-3), (INT13-1), (INT13-2), (INT13-3), (INTAX13-4), and (INTEQ-4):

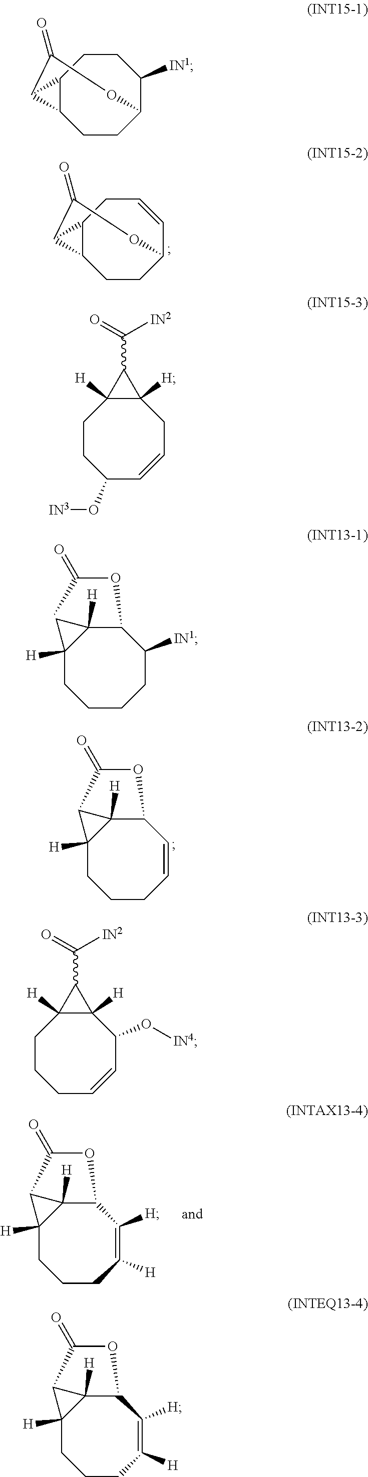

In Formulae (INT15-1) and (INT13-1) IN is a halogen; preferably iodine. In Formulae (INT15-3) and (INT13-3) IN² is —O—C₁₋₄ alkyl or —OH; preferably —O—CH₃.

In Formula (INT15-3) IN³ is hydrogen, C₁₋₄ alkyl, or acetyl; preferably hydrogen.

In Formula (INT13-3) IN⁴ is hydrogen, C₁₋₄ alkyl, or acetyl; preferably acetyl.

The enantiomers of the intermediates of Formulae (INT15-1), (INT15-2), (INT15-3), (INT13-1), (INT 13-2), (INT 13-3), (INTAX13-4), and (INTEQ13-4) are shown in Formulae (en-INT15-1), (en-INT15-2), (en-INT15-3), (en-INT13-1), (en-INT13-2), (en-INT13-3), (en-INTAX13-4), and (en-INTEQ13-4), respectively:

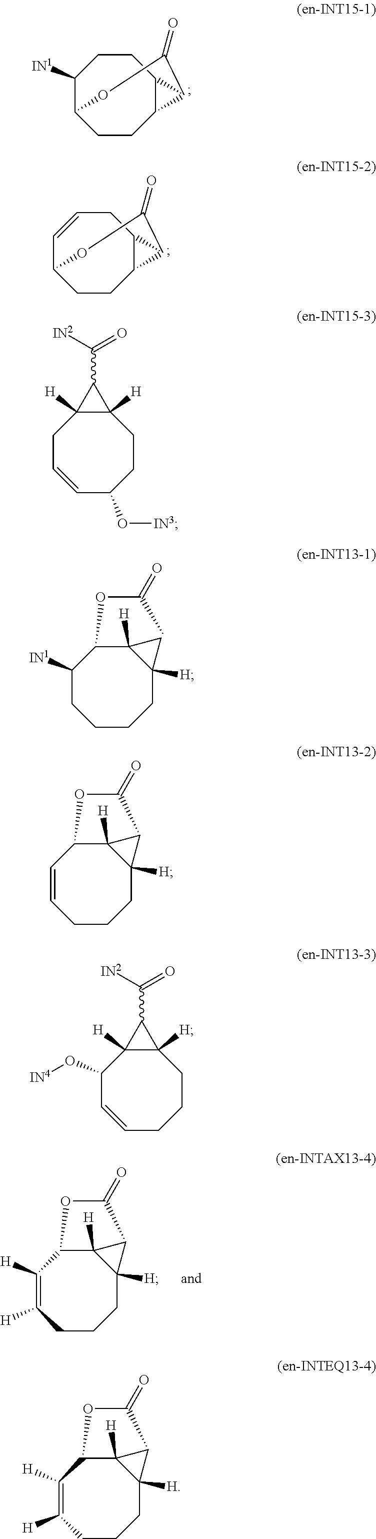

Preferred isomers of Formula (INT15-3) are shown below in Formulae (INT15-3EN) and (INT15-3EX):

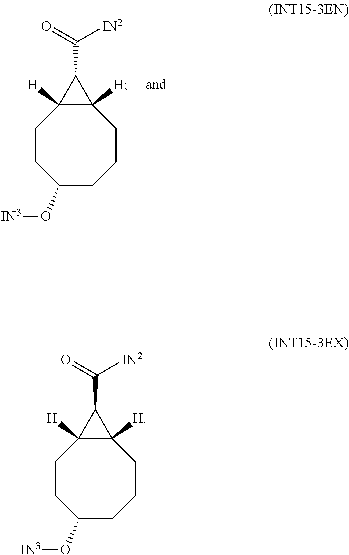

Enantiomers of the intermediates of Formulae (INT15-3EN) and (INT15-3EX) are depicted below in Formulae (en-INT15-3EN) and (en-INT15-3EX), respectively:

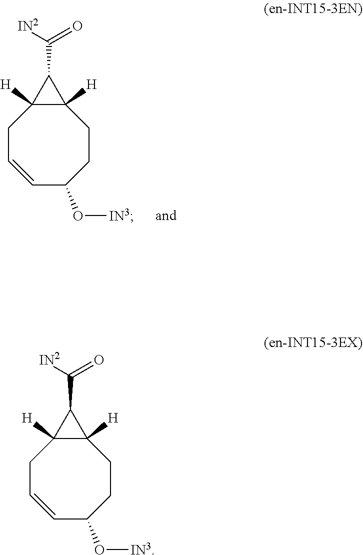

Preferred isomers of Formula (INT13-3) are shown below in Formulae (INT13-3EN) and (INT13-3EX):

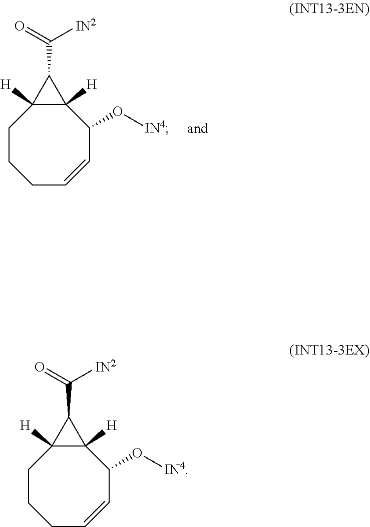

Enantiomers of the intermediates of Formulae (INT13-3EN) and (INT13-3EX) are depicted below in Formulae (en-INT13-3EN) and (en-INT13-3EX), respectively:

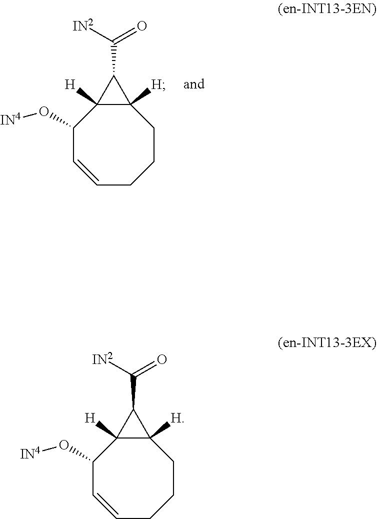

### Combinations

The disclosure also pertains to a combination of
(A1) a compound according to the disclosure, or the salt, solvate, hydrate, and/or enantiomer thereof, or
(A2) a composition according to the disclosure: with
(B) a diene or a salt, solvate, or hydrate thereof.

Preferably, the diene is a tetrazine. Preferred embodiments of dienes and tetrazines are further described below. The disclosure also relates to a combination of (A1) or (A2) with an Activator, or a salt, solvate, or hydrate thereof.

Components (A1), (A2), and (B) may be kept separately. Preferably, the combination of the disclosure is a kit. Preferably, in a kit one or more compounds of the disclosure and one or more dienes are provided in separate containers.

### Dienes

It will be understood that all dienes herein can be provided as a salt, hydrate, and/or solvate.

The Activator comprises a diene, preferably a tetrazine, more preferably a 1,2,4,5-tetrazine. The diene typically reacts with a dienophile. Preferably, the Activator is a tetrazine, more preferably a 1,2,4,5-tetrazine. Synthesis routes to tetrazines in general are readily available to the skilled person, based on standard knowledge in the art. References to tetrazine synthesis routes include for example Lions et al, J. Org. Chem., 1965, 30, 318-319; Horwitz et al, J. Am. Chem. Soc., 1958, 80, 3155-3159; Hapiot et al, New J. Chem., 2004, 28, 387-392, Kaim et al, Z. Naturforsch., 1995, 50b, 123-127; Yang et al., Angew. Chem. 2012, 124, 5312-5315; Mao et al., Angew. Chem. Int. Ed. 2019, 58, 1106-1109; Qu et al. Angew. Chem. Int. Ed. 2018, 57, 12057-12061; Selvaraj et al., Tetrahedron Lett. 2014, 55, 4795-4797; Fan et al., Angew. Chem. Int. Ed. 2016, 55, 14046-14050.

Preferably, the diene is a tetrazine satisfying Formula (4) or a salt, solvate, or hydrate thereof:

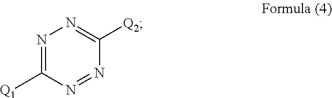

wherein each moiety Q₁ and Q₂ is independently selected from the group consisting of hydrogen, organic molecules, and inorganic molecules; and preferably at least one of moieties Q₁ and Q₂ is not hydrogen. Preferably, each moiety Q₁ and Q₂ is independently selected from RG1, RG3, RG4, and RG5.

Preferably, the tetrazine is symmetrical, i.e. Q₂ equals Q₁. This is advantageous, as it typically simplifies the synthesis of the tetrazine.

Preferably, in Formula (4) Q₁ and Q₂ are selected from the group of hydrogen, (cyclo)alkyl, (cyclo)alkenyl, (cyclo)alkynyl, hetero(cyclo)alkyl, hetero(cyclo)alkenyl, hetero(cyclo)alkynyl, aryl, heteroaryl, linear or cyclic vinyl ethers, and combinations thereof, and Q₁ and Q₂ not being hydrogen are optionally substituted, preferably with one or more moieties according to RG1 not being hydrogen. Preferably, each individual Q₁ and Q₂ group comprises at most 4 substituents, more preferably at most 3 substituents, even more preferably at most 2 substituents, and most preferably at most 1 substituent. Preferably, in relation to Formula (4) aryl is phenyl.

In some embodiments, the diene is a multimeric compound, comprising a plurality of tetrazines. These multimeric compounds can be peptide, peptoid, protein, oligonucleotide, oligosaccharide, polymersome, biomolecules, polymers, dendrimers, liposomes, micelles, particles, nanoparticles, microparticles, polymer particles, or other polymeric constructs. If the diene is a multimeric compound, it is preferred that it comprises a tetrazine coupled, optionally via a spacer, to a polymer, more preferably hyaluronic acid.

In Formula (4), the Q₁ and Q₂ are optionally bound to a moiety according to RG3 or RG4, preferably RG3, more preferably a polymer or a protein.

In some embodiments, the Q₁ and Q₂ not being hydrogen are not substituted.

Preferably, in Formula (4):
(a) Q₁ and Q₂ are independently selected from the group consisting of 2-pyridyl, 3-pyridyl, and 4-pyridyl;
(b) Q₁ is selected from the group consisting of 2,6-pyrimidyl, 2,5-pyrimidyl, 3,5-pyrmidyl, and 2,4-pyrimidyl; and Q₂ is (hetero)alkyl; or
(c) Q₁ is phenyl and Q₂ is hydrogen;
(d) Q₁ is phenyl and Q₂ is phenyl;
(e) Q₁ is phenyl and Q₂ is C₁-C₈ (hetero)alkyl;
(f) Q₁ and Q₂ are C₁-C₈ (hetero)alkyl;
(g) Q₁ and Q₂ are C₃-C₈ (cyclo)alkenyl;
(h) Q₁ and Q₂ are vinyl ether, preferably cyclic vinyl ether;
and in (a)-(h) all Q₁ and Q₂ not being hydrogen are optionally substituted by a group according to RG1 not being hydrogen.

Preferably, Q₁ in Formula (4) is selected from the group consisting of phenyl, vinyl ether, and C₃-C₅ heteroaryl, and is optionally further substituted with at least one moiety RG5, preferably not more than two, more preferably not more than one moiety RG5. Herein, in relation to Formula (4) preferred heteroaryls are 2-pyridyl, 3-pyridyl, 4-pyridyl, 2,6-pyrimidyl, 3,5-pyrimidyl, 2,5-pyrimidyl, 2,4-pyrimidyl, 2,4 imidazyl, 2,5 imidazyl, 2,3-pyrazyl, 3,4-pyrazyl, oxazol, isoxazol, thiazol, oxazoline, 2-pyrryl, 3-pyrryl, 2-thiophene, and 3-thiophene. More preferably, in relation to Formula (4) heteroaryl is 2-pyridyl, 3-pyridyl, 4-pyridyl, 2,6-pyrimidyl, 2,5-pyrimidyl, 3,5-pyrmidyl, or 2,4-pyrimidyl.

Particularly preferred dienes are those of Formula (4) wherein Q₁ and Q₂ are selected from the group consisting of phenyl, (hetero)alkyl, and linear or cyclic vinyl ether, and wherein at least one of Q₁ and Q₂ is attached to a Targeting Agent, preferably an antibody.

These dienes can be advantageously used to prelocalize the diene prior to administrating a compound of the disclosure, preferably a Prodrug according to the disclosure.

Preferably, Q₁ in Formula (4) is C₃-C₅ heteroaryl or vinyl ether, and is optionally further substituted with at least one moiety RG5, preferably not more than two, more preferably not more than one moiety RG5, and Q₂ is C₃-C₅ heteroaryl or vinyl ether, and is optionally further substituted with one or more moieties RG5, preferably not more than two, more preferably not more than one moiety RG5. Herein, preferred heteroaryls are 2-pyridyl, 3-pyridyl, 4-pyridyl, 2,6-pyrimidyl, 3,5-pyrimidyl, 2,5-pyrimidyl, 2,4-pyrimidyl, 2,4 imidazyl, 2,5 imidazyl, 2,3-pyrazyl, 3,4-pyrazyl, oxazol, isoxazol, thiazol, oxazoline, 2-pyrryl, 3-pyrryl, 2-thiophene, and 3-thiophene.

In some embodiments, Q₁ in Formula (4) is C₃-C₅ heteroaryl, and is optionally further substituted with at least one moiety RG5, preferably not more than two, more preferably not more than one moiety RG5, and Q₂ is H. In some embodiments, Q₁ in Formula (4) is a phenyl ring, and is optionally further substituted with at least one moiety RG5, preferably not more than two, more preferably not more than one moiety RG5, and Q₂ is —H. In some embodiments, Q₁ in Formula (4) is a phenyl ring, and is optionally further substituted with at least one moiety RG5, preferably not more than two, more preferably not more than one moiety RG5, and Q₂ is a phenyl ring, and is optionally further substituted with at least one moiety RG5, preferably not more than two, more preferably not more than one moiety RG5.

In some embodiments, Q₁ in Formula (4) is a phenyl ring, and is optionally further substituted with at least one moiety RG5, preferably not more than two, more preferably not more than one moiety RG5, and Q₂ is selected from the group consisting of C₆ aryl, and C₃₋₅ heteroaryl, and is optionally further substituted with at least one moiety RG5, preferably not more than two, more preferably not more than one moiety RG5. In some embodiments, Q₁ in Formula (4) is C₁-C₁₂ (hetero)alkyl, and is optionally further substituted with at least one moiety RG5, preferably not more than two, more preferably not more than one moiety RG5, and Q₂ selected from the group consisting of C₆ aryl, and C₃₋₅ heteroaryl, and is optionally further substituted with at least one moiety RG5, preferably not more than two, more preferably not more than one moiety RG5. In some embodiments, Q₁ in Formula (4) is C₁-C₁₂ (hetero)alkyl, and is optionally further substituted with at least one moiety RG5, preferably not more than two, more preferably not more than one moiety RG5, and Q₂ in Formula (4) is C₁-C₁₂ (hetero)alkyl, and is optionally further substituted with at least one moiety RG5, preferably not more than two, more preferably not more than one moiety RG5. In some embodiments, Q₁ in Formula (4) is C₃-C₁₂ (hetero)alkenyl, and is optionally further substituted with at least one moiety RG5, preferably not more than two, more preferably not more than one moiety RG5, and Q₂ in Formula (4) is C₁-C₁₂ (hetero)alkenyl, and is optionally further substituted with at least one moiety RG5, preferably not more than two, more preferably not more than one moiety RG5.

Preferably, the tetrazine satisfies Formula (4x):

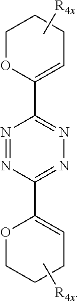

Herein, each R₄ₓ is independently according to RG1, preferably —(S^{P})ᵢ—C^{B}. Preferably, the C^{B} in R₄ₓ is a Targeting Agent as defined herein. Preferably, the C^{B} in R₄ₓ is a protein or a peptide, more preferably an antibody, or an antibody fragment, wherein preferably the antibody fragment is a diabody, nanobody, of minibody. Most preferably, the C^{B} in R₄ₓ is the antibody CC49 or the diabody AVP0458.

Preferably, the S^{P} in R₄ₓ is coupled to the vinyl ether ring via a moiety —C₁0.3 (hetero)alkylene-O—, more preferably —CH₂—O—.

Preferably, the S^{P} in R₄ₓ comprises a polymer, more preferably polyethylene glycol (PEG). Preferably, the S^{P} in R₄ₓ comprises or is a moiety —C₁₋₃ (hetero)alkylene-(R4ₓ₁—CH₂—(CH₂—O—CH₂)ₚ₁—CH₂)ₚ₂—R₄ₓ₂. Therein, p1 is an integer of from 1 to 24, preferably of from 5 to 15, more preferably of from 8 to 10, and p2 is 0 or 1. More preferably, the S^{P} in R₄ₓ comprises or is a moiety —CH₂—(R₄ₓ₁—CH₂—(CH₂—O—CH₂)ₚ₁—CH₂)ₚ₂—R₄ₓ₂.

R₄ₓ₁ is according to RG2a, RG2b, or RG2c, and is preferably selected from the group consisting of —OC(O)—, —C(O)O—, —OC(O)—NH—, —NH—C(O)O—, —OC(S)—, —C(S)O—, —OC(S)—NH—, —NH—C(S)O—, —NHC(O)—, —C(O)NH—, —NHC(S)—, —C(S)NH—, —NHC(O)O—, —O—C(O)NH—, —NHC(S)O—, —O—C(S)NH—, —NHC(O)NH—, and —NHC(S)NH—. Preferably, R₄ₓ₁ is —OC(O)—NH—.

R₄ₓ₂ is —C(O)—, —C(S)—, —OC(O)—, —OC(S)—, —OC(O)—NH—, OC(S)—NH—, —NH—, —NHC(O)—, —NHC(S)—, NHC(O)NH—, —NHC(S)NH—, —OC(O)—NH—C₁₋₃ (hetero)alkylene-, or —NH—C(O)—C₁₋₃ (hetero)alkylene-, —OC(O)—NH—C₁₋₃ (hetero)alkylene-R₄ₓ₃, or —NH—C(O)—C₁₋₃ (hetero)alkylene-R₄ₓ₃. It will be understood that R₄ₓ₂ is coupled to the C^{B}, optionally via another spacer. Preferably, the C₁₋₃ (hetero)alkylene is a C₁ (hetero)alkylene. Preferably, if p1 is 0, then R₄ₓ₂ is —OC(O)—, —NHC(O)—, —OC(O)—NH—C₁₋₃ (hetero)alkylene-R₄ₓ₃, or —NH—C(O)—C₁₋₃ (hetero)alkylene-R₄ₓ₃.

R₄ₓ₃ is according to RG2a, RG2b, or RG2c, preferably RG2b. More preferably, R₄ₓ₃ is

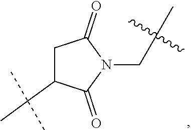

wherein the wiggly line indicates a bond to the C₁₋₃ (hetero)alkylene and the dashed line a bond to the C^{B}.

Tetrazines of Formula (4x) are particularly advantageous, as they have excellent stability and reactivity, also in physiological conditions. As such, tetrazines according to Formula (4x) are particularly useful in embodiments wherein the tetrazine of Formula (4x) is administered to the subject prior to administering the dienophile as described herein. Then, the tetrazine of Formula (4x), which comprises a Targeting Agent, can first accumulate at the target site, after which the dienophile can be administered to said subject. The dienophile then does not necessarily comprise a Targeting Agent, but is simply only unmasked at the target site after reaction with the tetrazine of Formula (4x) that is accumulated at said target site. Preferably, the tetrazine according to Formula (4x) is a tetrazine according to Formula (4x1):

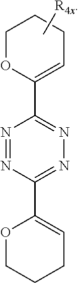

Most preferably, the tetrazine according to Formula (4x) is a tetrazine according to Formula (4x2):

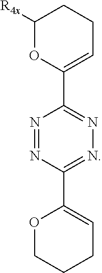

In some embodiments, the tetrazine is in accordance with any one of the Formulae (6)-(13):

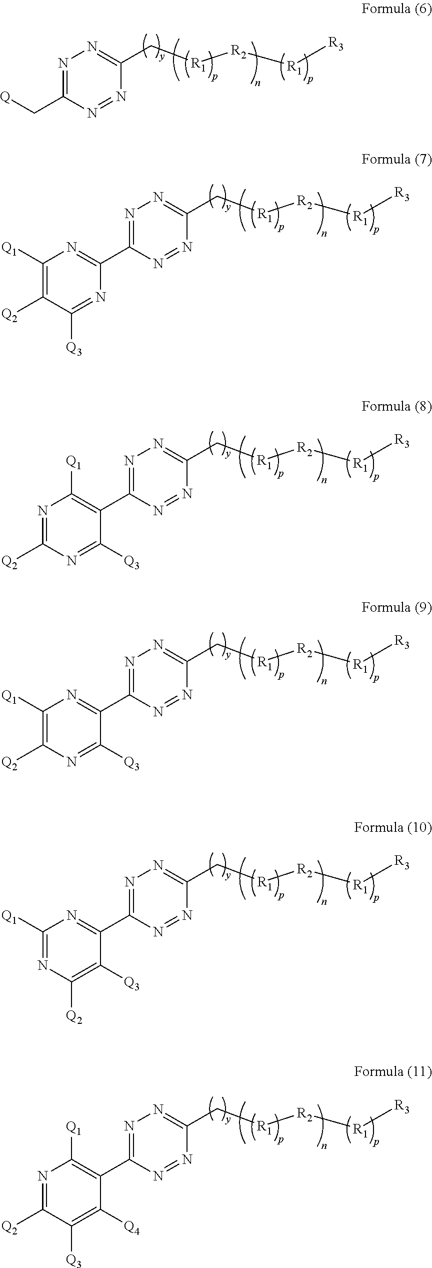

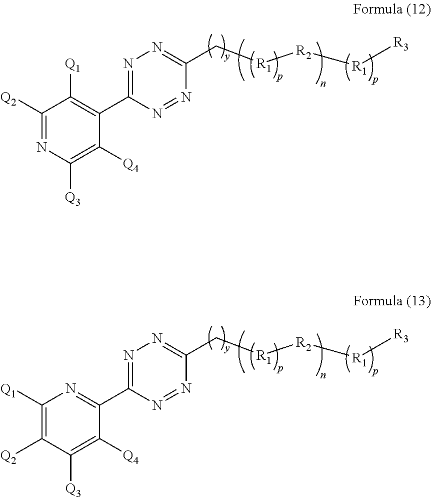

wherein each moiety Q, Q₁, Q₂, Q₃, and Q₄ is independently selected from the group consisting of hydrogen and moieties according to RG5 as defined herein. For the moiety —(CH₂)_{y}—((R₁)ₚ—R₂)ₙ—(R₁)ₚ—R₃; y is defined as for z of RG5; R₁ is as defined for R₁₀ of RG5; p is as defined as for h of RG5; R₂ is as defined as for R₁₁ of RG5; n is as defined as for n of RG5; and R₃ is as defined as for R₁₂ of RG5.

Preferably, in any one of Formulae (6), (7), (8), (9), (10), (11), (12), and (13), at least one moiety selected from the group consisting of Q, Q₁, Q₂, Q₃, and Q₄ is hydrogen.

Preferably, in any one of Formulae (7), (8), (9), (10), (11), (12), and (13), at least two, more preferably at least three, most preferably all, moieties selected from the group consisting of Q₁, Q₂, Q₃, and Q₄ are hydrogen.

Preferably, for all compounds disclosed herein comprising a group Q, Q₁, Q₂, Q₃, Q₄ or —(CH₂)_{y}—((R₁)ₚ—R₂)ₙ—(R₁)ₚ—R₃, at least one of these has a molecular weight in a range of from 100 Da to 3000 Da. Preferably, at least one of these has a molecular weight in a range of from 100 Da to 2000 Da. More preferably, at least one of these has a molecular weight in a range of from 100 Da to 1500 Da, even more preferably in a range of from 150 Da to 1500 Da. Even more preferably still, at least one of these has a molecular weight in a range of from 150 Da to 1000 Da, most preferably in a range of from 200 Da to 1000 Da.

Preferably, for all compounds disclosed herein comprising a group Q, Q₁, Q₂, Q₃, Q₄ or —(CH₂)_{y}—((R₁)ₚ—R₂)ₙ—(R₁)ₚ—R₃, none of these groups has a molecular weight of more than 3000 Da, in particular in the case the Activator needs to efficiently extravasate into tissues.

On the other hand, when the Activator is meant to stay in circulation, for example when the Activator is a Clearing Agent or a Cleaving Agent, it is preferred that for all compounds disclosed herein comprising a group Q, Q₁, Q₂, Q₃, Q₄ or —(CH₂)—((R₁)ₚ—R₂)ₙ—(R₁)ₚ—R₃, one or more of these groups has a molecular weight of more than 3000 Da.

### Formula (14)

In some embodiments, the diene is a tetrazine satisfying Formula (14):

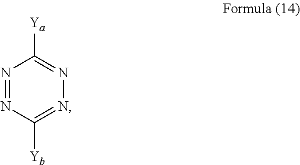

and salts, solvates, and hydrates thereof, wherein Yₐ is selected from the group consisting of Y₁, Y₂, Y₃, Y₄, Y₅, Y₆, Y₇, and Y₈:

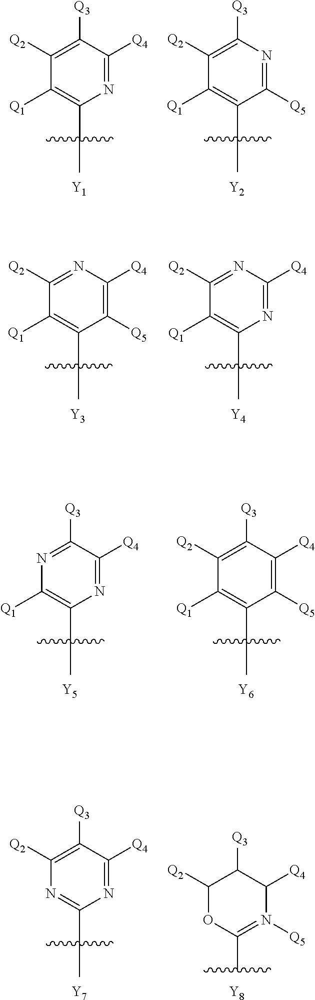

In Formula (14), Y_{b} is according to RG1, and is preferably selected from the group consisting of Y₁, Y₂, Y₃, Y₄, Y₅, Y₆, Y₇, Y₈, hydrogen, X₄₇, and —(S^{P})ᵢ—C^{B}; wherein i is 0 or 1. For Formula (14), each Q₁ and Q₅, are independently selected from the group consisting of X₄₅, hydrogen, X₄₇ and —(S^{P})ᵢ—C^{B}; each Q₂ and Q₄, are independently selected from the group consisting of X₄₆, hydrogen, X₄₇, and —(S^{P})ᵢ—C^{B}; each Q₃ is independently selected from the group consisting of hydrogen, X₄₇, and —(S^{P})ᵢ—C^{B}.

Preferably, the compound of Formula (14) comprises at least one X₄₅ or X₄₆ group.

Each X₄₅ is independently selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁, SO₃H, and PO₃H₂.

Each X₄₆ is independently selected from the group consisting of N(X₅₀)₂, C(X₅₁)₂N(X₅₀)₂, NX₅₀C(O)X₅₁, NX₅₀C(S)X₅₁, OH, SH, C(O)OH, C(S)OH, C(O)SH, C(S)SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, NX₅₀OX₅₁, SO₃H, and PO₃H₂.

Each X₅₀ and X₅₁ is independently selected from the group consisting of hydrogen, X₄₈, and —(S^{P})ᵢ—C^{B}. Each X₄₈ is independently selected from the group consisting of hydrogen, C₁-C₄ (hetero)alkyl, C₂-C₄ (hetero)alkenyl, and C₄₋₆ (hetero)aryl; wherein for X₄₈ the (hetero)alkyl, (hetero)alkenyl, and (hetero)aryl are optionally substituted with a moiety selected from the group consisting of —Cl, —F, —Br, —I, —OH, —NH₂, ═O, —SH, —SO₃H, —PO₃H, —PO₄H₂, and —NO₂; and optionally contain at most two heteroatoms selected from the group consisting of —O—, —S—, —NH—, —P—, and —Si—, wherein the N, S, and P atoms are optionally oxidized. Preferably, X₅₀ is hydrogen.

Each X₄₇ is independently selected from the group consisting of —F, —Cl, —Br, —I, —OX₄₉, —N(X₄₉)₂, —SO₃, —PO₃⁻, —NO₂, —CF₃, —SX₄₉, S(═O)₂N(X₄₉)₂, OC(═O)X₄₉, SC(═O) X₄₉, OC(═S)X₄₉, SC(═S)X₄₉, NX₄₉C(═O)—X₄₉, NX₄₉C(═S)—X₄₉, NX₄₉C(═O)O—X₄₉, NX₄₉C(═S)O—X₄₉, NX₄₉C(═O)S—X₄₉, NX₄₉C(═S)S—X₄₉, OC(═O)N(X₄₉)₂, SC(═O)N(X₄₉)₂, OC(═S)N(X₄₉)₂, SC(═S)N(X₄₉)₂, NX₄₉C(═O)N(X₄₉)₂, NX₄₉C(═S)N(X₄₉)₂, C(═O)X₄₉, C(═S)X₄₉, C(═O)N(X₄₉)₂, C(═S)N(X₄₉)₂, C(═O)O—X₄₉, C(═O)S—X₄₉, C(═S)O—X₄₉, C(═S)S—X₄₉, —S(O)X₄₉, —S(O)₂X₄₉, NX₄₉S(O)₂X₄₉, —ON(X₄₉)₂, —NX₄₉OX₄₉, C₁-C₈ (hetero)alkyl, C₂-C₈ (hetero)alkenyl, C₂-C₈ alkynyl, C₆-C₁₂ aryl, C₂-C₁₂ heteroaryl, C₃-C₈ cycloalkyl, C₅-C₈ cycloalkenyl, C₃-C₁₂ (hetero)alkyl(hetero)aryl, C₃-C₁₂ (hetero)arylalkyl, C₄-C₁₂ (hetero)alkylcycloalkyl, C₄-C₁₂ cycloalkylalkyl, C₅-C₁₂ cycloalkyl(hetero)aryl, C₅-C₁₂ (hetero)arylcycloalkyl, and combinations thereof, wherein the (hetero)alkyl, (hetero)alkenyl, alkynyl, aryl, heteroaryl, cycloalkyl, cycloalkenyl, (hetero)alkyl(hetero)aryl, (hetero)arylalkyl, (hetero)alkylcycloalkyl, cycloalkylalkyl, cycloalkyl(hetero)aryl and (hetero)arylcycloalkyl are optionally substituted with a moiety selected from the group consisting of —Cl, —F, —Br, —I, —OX₄₉, —N(X₄₉)₂, —SO₃X₄₉, —PO₃(X₄₉)₂, —PO₄(X₄₉)₂, —NO₂, —CF₃, ═O, ═NX₄₉, and —SX₄₉.

Each X₄₉ is independently selected from the group consisting of hydrogen, C₁-C₈ (hetero)alkyl, C₂-C₈ (hetero)alkenyl, C₂-C₈ alkynyl, C₆-C₁₂ aryl, C₂-C₁₂ heteroaryl, C₃-C₅ cycloalkyl, C₅-C₈ cycloalkenyl, C₃-C₁₂ (hetero)alkyl(hetero)aryl, C₃-C₁₂ (hetero)arylalkyl, C₄-C₁₂ (hetero)alkylcycloalkyl, C₄-C₁₂ cycloalkylalkyl, C₅-C₁₂ cycloalkyl(hetero)aryl and C₅-C₁₂ (hetero)arylcycloalkyl, wherein X₄₉ not being hydrogen is optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH.

Preferably, at most two, more preferably at most one of Q₁, Q₂, Q₃, Q₄, and Q₅ are C^{B}; wherein the compound according to Formula (14) preferably comprises for each individual Yₐ and Y_{b} at most four C^{B}, more preferably at most two C^{B}, most preferably at most one C^{B}; wherein the compound according to Formula (14) preferably comprises at least one C^{B}; wherein preferably for each individual Yₐ and Y_{b} at most three, more preferably at most two of Q₁, Q₂, Q₃, Q₄, and Q₅ are not hydrogen; wherein preferably for each individual Yₐ and Y_{b} at most two of Q₁, Q₂, Q₃, Q₄, and Q₅ are X₄₅ or X₄₆, wherein preferably for each individual Yₐ and Y_{b} one of Q₁, Q₂, Q₄, and Q₅ is X₄₅ or X₄₆, wherein preferably both Yₐ and Y_{b} comprise at least one X₄₅ or X₄₆, wherein preferably both Yₐ and Y_{b} comprise one X₄₅ or X₄₆, wherein preferably both Yₐ and Y_{b} comprise one X₄₅ or X₄₆, whereby the X₄₅ comprised in Yₐ is the same as the X₄₅ comprised in Y_{b}, and/or the X₄₆ comprised in Yₐ is the same as the X₄₆ comprised in Y_{b}, wherein preferably Yₐ and Y_{b} are both independently selected Y₁, or both independently selected Y₂, or both independently selected Y₃, or both independently selected Y₄, or both independently selected Y₅; or both independently selected Y₇; or both independently selected Y₈.

Preferably, in Formula (14), when Q₁ is a X₄₇ or —(S^{P})ᵢ—C^{B}, then for Q₁ the X₄₇ and —(S^{P})ᵢ—C^{B} are not a group in accordance with the definition of X₄₅. Preferably, in Formula (14), when Q₅ is a X₄₇ or —(S^{P})ᵢ—C^{B}, then for Q₅ the X₄₇ and —(S^{P})ᵢ—C^{B} are not a group in accordance with the definition of X₄₅. Preferably, in Formula (14), when Q₂ is a X₄₇ or —(S^{P})ᵢ—C^{B}, then for Q₂ the X₄₇ and —(S^{P})ᵢ—C^{B} are not a group in accordance with the definition of X₄₆. Preferably, in Formula (14), when Q₄ is a X₄₇ or —(S^{P})ᵢ—C^{B}, then for Q₄ the X₄₇ and —(S^{P})ᵢ—C^{B} are not a group in accordance with the definition of X₄₆. Preferably Yₐ equals Y_{b}.

Preferably Yₐ is selected from Y₁, Y₂, Y₃, Y₄ or Y₅ and Y_{b} is hydrogen, X₄₇ or —(S^{P})ᵢ—C^{B}. Preferably Yₐ is selected from Y₁, Y₂, Y₃, Y₄ or Y₅ and Y_{b} is hydrogen. Preferably the compound according to Formula (14) does not comprise —(S^{P})ᵢ—C^{B}.

Preferably when X₄₅ or X₄₆ is N(X₅₀)₂, then one X₅₀ is hydrogen and one X₅₀ is X₄₈ or —(S^{P})ᵢ—C^{B}. Preferably Formula (14) does not comprise X₄₆. Preferably, both Q₁ in Formula (14) are X₄₅. Preferably, both Q₂ in Formula (14) are X₄₆. Preferably, both Q₅ in Formula (14) are X₄₅. Preferably, both Q₄ in Formula (14) are X₄₆.

Preferably, each X₄₅ is independently selected from the group consisting of N(X₅₀)₂, NX₅₀C(O)X₅₁, NX₅₀C(S)X₅₁, OH, SH, NX₅₀C(O)OX₅₁, NX₅₀C(S)OX₅₁, NX₅₀C(O)SX₅₁, NX₅₀C(S)SX₅₁, NX₅₀C(O)N(X₅₁)₂, NX₅₀C(S)N(X₅₁)₂, NX₅₀SO₂X₅₁, NX₅₀SO₃X₅₁, and NX₅₀OX₅₁. More preferably, each X₄₅ is independently selected from the group consisting of N(X₅₀)₂, NX₅₀C(O)X₅₁, NX₅₀C(S)X₅₁, OH and SH.

In some embodiments, X₄₅ is selected from the group consisting of NHX₅₀, C(X₅₁)₂NH₂, CHX₅₁NH₂, CH₂N(X₅₀)₂, CH₂NHX₅₀, NHC(O)X₅₁, NHC(S)X₅₁, OH, and SH. In some embodiments, X₄₅ is NHX₅₀. In some embodiments, X₄₅ is C(X₅₁)₂NH₂. In some embodiments, X₄₅ is CHX₅₁NH₂. In some embodiments, X₄₅ is CH₂N(X₅₀)₂. In some embodiments, X₄₅ is CH₂NHX₅₀. In some embodiments, X₄₅ is NH₂. In some embodiments, X₄₅ is CH₂NH₂. In some embodiments, X₄₅ is NHC(O)X₅₁. In some embodiments, X₄₅ is NHC(S)X₅₁. In some embodiments, X₄₅ is OH. In some embodiments, X₄₅ is SH.

Preferably, X₄₆ is independently selected from the group consisting of N(X₅₀)₂, NX₅₀C(O)X₅₁, NX₅₀C(O)OX₅₁, and NX₅₀C(O)N(X₅₁)₂. More preferably, X₄₆ is selected from the group consisting of N(X₅₀)₂, and NX₅₀C(O)X₅₁. Most preferably, X₄₆ is selected from the group consisting of NHX₅₀ and NHC(O)X₅₁. In some embodiments, X₄₆ is NHX₅₀. In some embodiments, X₄₆ is NH₂. In some embodiments, X₄₆ is NHC(O)X₅₁.

Preferably, each X₄₇ is independently selected from the group consisting of F, —OH, —NH₂, —SO₃⁻, —NO₂, —CF₃, —SH, C₁-C₆ (hetero)alkyl, C₆ aryl, C₄-C₅ heteroaryl, C₅-C₈ (hetero)alkyl(hetero)aryl, C₅-C₈ (hetero)arylalkyl, C₄-C₈ (hetero)alkylcycloalkyl, and C₄-C₈ cycloalkylalkyl. In a more preferred embodiment, each X₄₇ is independently selected from the group consisting of F, —SO₃⁻, —NO₂, —CF₃, C₁-C₆ (hetero)alkyl, C₆ aryl, C₄-C₅ heteroaryl, C₅-C₅ (hetero)alkyl(hetero)aryl, C₅-C₈ (hetero)arylalkyl, C₄-C₈ (hetero)alkylcycloalkyl, and C₄-C₈ cycloalkylalkyl. Preferably, the X₄₇ substituents do not contain heteroatoms. Preferably, X₄₇ is not substituted. In another preferred embodiment, X₄₇ does not contain heteroatoms.

Preferably, each X₄₉ is independently selected from the group consisting of hydrogen, C₁-C₄ (hetero)alkyl, C₂-C₄ (hetero)alkenyl, and C₄₋₆ (hetero)aryl. For X₄₉ the (hetero)alkyl, (hetero)alkenyl, and (hetero)aryl are optionally substituted with a moiety selected from the group consisting of —Cl, —F, —Br, —I, —OH, —NH₂, ═O, —SH, —SO₃H, —PO₃H, —PO₄H₂ and —NO₂; and optionally contain at most two heteroatoms selected from the group consisting of —O—, —S—, —NH—, —P—, and —Si—, wherein the N, S, and P atoms are optionally oxidized.

Preferably, X₄9 is C₁-C₄ (hetero)alkyl. Preferably, the X₄₈ substituents do not contain heteroatoms. Preferably, X₄₉ is not substituted. In another preferred embodiment, X₄₈ does not contain heteroatoms.

Preferably, X₄₉ is selected from the group consisting of hydrogen, C₁-C₈ (hetero)alkyl, C₂-C₈ (hetero)alkenyl, C₂-C₈ (hetero)alkynyl, C₆-C₁₂ aryl, C₂-C₁₂ heteroaryl, C₃-C₅ (hetero)cycloalkyl, C₅-C₈ (hetero)cycloalkenyl, C₃-C₁₂ (hetero)alkyl(hetero)aryl, C₃-C₁₂ (hetero)arylalkyl, C₄-C₁₂ (hetero)alkylcycloalkyl, C₄-C₁₂ cycloalkylalkyl, C₅-C₁₂ cycloalkyl(hetero)aryl and C₅-C₁₂ (hetero)arylcycloalkyl, wherein X₄₉ not being hydrogen is optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH.

Preferably, X₄₉ is selected from the group consisting of hydrogen, C₁-C₄ (hetero)alkyl, C₂-C₄ (hetero)alkenyl, C₂-C₄ (hetero)alkynyl, C₆-C₈ aryl, C₂-C₈ heteroaryl, C₃-C₆ cycloalkyl, C₅-C₆ cycloalkenyl, C₃-C₁₀ (hetero)alkyl(hetero)aryl, C₃-C₁₀ (hetero)arylalkyl, C₄-C₈ (hetero)alkylcycloalkyl, C₄-C₈ cycloalkylalkyl, C₅-C₁₀ cycloalkyl(hetero)aryl and C₅-C₁₀ (hetero)arylcycloalkyl, wherein the X₄₉ not being hydrogen are optionally substituted with a moiety selected from the group consisting of —Cl, —F, —Br, —I, —OH, —NH₂, —SO₃H, —PO₃H, —PO₄H₂, —NO₂, —CF₃, ═O, ═NH, and —SH, and optionally contain one or more heteroatoms selected from the group consisting of O, S, NH, P, and Si, wherein the N, S, and P atoms are optionally oxidized, wherein the N atoms are optionally quaternized.

Preferably, X₄₉ substituents do not contain heteroatoms. Preferably, X₄₉ is not substituted. In another preferred embodiment, X₄₉ does not contain heteroatoms.

Preferably, each X₅₀ is independently selected from the group consisting of hydrogen, X₄₈, and —(S^{P})ᵢ—C^{B}. In some embodiments, X₅₀ is X₄₉. In some embodiments, X₅₀ is —(S^{P})ᵢ—C^{B}. Preferably, X₅₀ is H.

Preferably, each X₅₁ is independently selected from the group consisting of hydrogen, X₄₈, and —(S^{P})ᵢ—C^{B}. In some embodiments, X₅₁ is X₄₉. In some embodiments, X₅₁ is —(S^{P})ᵢ—C^{B}. Preferably, X₅₁ is H.

Preferably, in Formula (14) Q₁ is selected from the group consisting of hydrogen, X₄₇, and —(S^{P})ᵢ—C^{B}. In some embodiments, Q₁ in Formula (14) is hydrogen. In some embodiments, Q₁ in Formula (14) is X₄₇. Preferably, Q₁ in Formula (14) is —(S^{P})ᵢ—C^{B}, and preferably Q₂, Q₃, Q₄, Q₅, Q₆, Q₇, Q₈, Q₉, and Q₁₀ are X₄₅, X₄₆, or hydrogen.

Preferably, Q₂ in Formula (14) is selected from the group consisting of hydrogen, X₄₇, and —(S^{P})ᵢ—C^{B}. In some embodiments, Q₂ in Formula (14) is hydrogen. In some embodiments, Q₂ is in Formula (14) X₄₇. Preferably, Q₂ in Formula (14) is —(S^{P})ᵢ—C^{B}, and preferably Q₁, Q₃, Q₄, Q₅, Q₆, Q₇, Q₈, Q₉, and Q₁₀ are X₄₅, X₄₆, or hydrogen.

Preferably, Q₃ in Formula (14) is selected from the group consisting of hydrogen, X₄₇, and —(S^{P})ᵢ—C^{B}. In some embodiments, Q₃ in Formula (14) is hydrogen. In some embodiments, Q₃ in Formula (14) is X₄₇. Preferably, Q₃ in Formula (14) is —(S^{P})ᵢ—C^{B}, and preferably Q₁, Q₂, Q₄, Q₅, Q₆, Q₇, Q₈, Q₉, and Q₁₀ are X₄₅, X₄₆, or hydrogen.

Preferably, Q₄ in Formula (14) is selected from the group consisting of hydrogen, X₄₇, and —(S^{P})ᵢ—C^{B}. In some embodiments, Q₄ in Formula (14) is hydrogen. In some embodiments, Q₄ in Formula (14) is X₄₇. Preferably, Q₄ in Formula (14) is —(S^{P})ᵢ—C^{B}, and preferably Q₁, Q₂, Q₃, Q₅, Q₆, Q₇, Q₅, Q₉ and Q₁₀ are X₄₅, X₄₆, or hydrogen.

Preferably, Q₅ in Formula (14) is selected from the group consisting of hydrogen, X₄₇, and —(S^{P})ᵢ—C^{B}. In some embodiments, Q₅ in Formula (14) is hydrogen. In some embodiments, Q₅ in Formula (14) is X₄₇. Preferably, Q₅ is —(S^{P})ᵢ—C^{B}, and preferably Q₁, Q₂, Q₃, Q₄, Q₆, Q₇, Q₅, Q₉ and Q₁₀ are X₄₅, X₄₆, or hydrogen.

Preferably, Q₆ in Formula (14) is selected from the group consisting of hydrogen, X₄₇, and —(S^{P})ᵢ—C^{B}. In some embodiments, Q₆ in Formula (14) is hydrogen. In some embodiments, Q₆ in Formula (14) is X₄₇. Preferably, Q₆ in Formula (14) is —(S^{P})ᵢ—C^{B}, and preferably Q₁, Q₂, Q₃, Q₄, Q₅, Q₇, Q₅, Q₉ and Q₁₀ are X₄₅, X₄₆, or hydrogen.

Preferably, Q₇ in Formula (14) is selected from the group consisting of hydrogen, X₄₇, and —(S^{P})ᵢ—C^{B}. In some embodiments, Q₇ in Formula (14) is hydrogen. In some embodiments, Q₇ in Formula (14) is X₄₇. Preferably, Q₇ in Formula (14) is —(S^{P})ᵢ—C^{B}, and preferably Q₁, Q₂, Q₃, Q₄, Q₅, Q₆, Q₈, Q₉ and Q₁₀ are X₄₅, X₄₆, or hydrogen.

Preferably, Q₈ in Formula (14) is selected from the group consisting of hydrogen, X₄₇, and —(S^{P})ᵢ—C^{B}. In some embodiments, Q₈ in Formula (14) is hydrogen. In some embodiments, Q₈ in Formula (14) is X₄₇. Preferably, Q₈ in Formula (14) is —(S^{P})ᵢ—C^{B}, and preferably Q₁, Q₂, Q₃, Q₄, Q₅, Q₆, Q₇, Q₉ and Q₁₀ are X₄₅, X₄₆, or hydrogen.

Preferably, Q₉ in Formula (14) is selected from the group consisting of hydrogen, X₄₇, and —(S^{P})ᵢ—C^{B}. In some embodiments, Q₉ in Formula (14) is hydrogen. In some embodiments, Q₉ in Formula (14) is X₄₇. Preferably, Q₉ in Formula (14) is —(S^{P})ᵢ—C^{B}, and preferably Q₁, Q₂, Q₃, Q₄, Q₅, Q₆, Q₇, Q₈ and Q₁₀ are X₄₅, X₄₆, or hydrogen.

Preferably, Q₁₀ in Formula (14) is selected from the group consisting of hydrogen, X₄₇, and —(S^{P})ᵢ—C^{B}. In some embodiments, Q₁₀ in Formula (14) is hydrogen. In some embodiments, Q₁₀ in Formula (14) is X₄₇. Preferably, Q₁₀ in Formula (14) is —(S^{P})ᵢ—C^{B}, and preferably Q₁, Q₂, Q₃, Q₄, Q₅, Q₆, Q₇, Q₅ and Q₉ are X₄₅, X₄₆, or hydrogen.

For Y₈, it is preferred that Q₂, Q₃, Q₄ are hydrogen and Q₁ is as defined for R₄ₓ.

### Dienes for Payload Release

Also dienes can be provided from which a payload is released upon reaction with a compound of the disclosure. Such dienes are described in, inter alia, WO 2018/004338 and Wang et al., Organic Letters 2022, volume 25, pages 5293-5297; both references are incorporated herein by reference.

Preferably, dienes for payload release have a structure according to Formula (15):

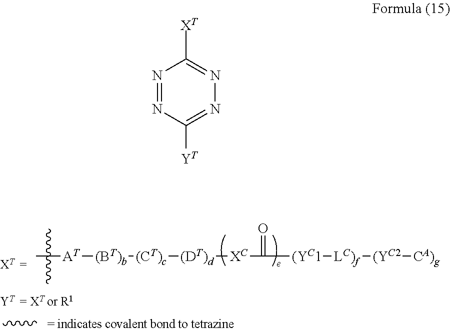

In Formula (15), Y^{T} is as defined for Y_{b} in relation to Formula (14).

In Formula (15), X^{T} is (i) -A^{T}-(B^{T})_{b}—(X^{C}—C(O))ₑ—(S^{P})ⱼ—C^{A} or (ii) —C(R₅₀)₂—(S^{P})ⱼ—C^{A}; wherein C^{A} is Construct A, which is a payload; j is 0 or 1; wherein b is 0 or 1; wherein e is 0 or 1; when X^{T} is (i), —(S^{P})ⱼ—C^{A} is connected to the remainder of X^{T} via O, S, secondary N, or a tertiary N, that is part of —(S^{P})ⱼ—C^{A}; when X^{T} is (ii), —(S^{P})ⱼ—C^{A} is connected to the remainder of X^{T} via O, S, or OC(O), that is part of —(S^{P})ⱼ—C^{A}.

In Formula (15), preferably j is 0.

In Formula (15), Construct A is preferably as defined herein, and more preferably C^{A} i an organic molecule or an inorganic molecule; and even more preferably C^{A} is a drug, protein, peptide, a chelating moiety, or toxin; wherein preferably the chelating moiety chelates a radionuclide.

In Formula (15), S^{P} is a spacer, preferably as defined herein, and most preferably S^{P} is a self-immolative linker, preferably as defined herein.

In Formula (15), A^{T} and B^{T}, are each independently are selected from the group consisting of CR²₂, C═O, C═CR³₂, C═NR⁶, NR⁴, O, S, S(═O), or S(═O)₂, provided that no sets consisting of adjacent atoms are present selected from the group consisting of —O—O—, —S—N(R⁴)—, —O—S—, —O—S(═O)—, —O—S(═O)₂—, and —S—S—, and wherein one R₂ or R₄ group from B^{T} and one R₂ or R₄ group from A^{T} can together be a bond, so as to form a double bond, and wherein two or more groups selected from R², R³, R⁴, R⁵, R⁶ can together form a C₆-C₁₂ aryl, or a 3- to 8-membered heterocycle; wherein when b is 0, A^{T} is CR²₂; wherein when b is 1, B^{T} is CR²₂.

In Formula (15), X^{C} is O, S, or NR⁶.

In Formula (15), each of R¹, R², R³, R⁴, R⁵, R⁶, and R₅₀ is individually selected from RG1 as defined herein.

In Formula (15), preferably each R¹, R², R³ and R⁵ is independently selected from the group consisting of H, (hetero)alkyl, aryl, heterocycle, carbocycle, F, CF₃, CF₂—R′, OR′, SR′, CN, C(═O)R′, S(═O)₂R″, S(═O)₂OR′, OS(═O)₂R″, PO₃R′₂, Si—R″₃, Si—O—R″₃, B(OR′)₂, S(═O)₂NR′₂, NR'S(═O)₂R″, C(═O)NR′₂, C(═S)NR′₂, NR′₂, NR″₃⁺, NR′C(═O)R′, NR′C(═S)R′, NR′C(═O)NR′₂, NR′C(═S)NR′₂ wherein each R′ is independently selected from the group consisting of H, (hetero)alkyl, aryl, carbocycle, heterocycle, and each R″ is independently selected from the group consisting of (hetero)alkyl, aryl, carbocycle, heterocycle, wherein all (hetero)alkyl, aryl, carbocycle, heterocycle moieties comprised in R¹, R², R³ and R⁵ can be unsubstituted or substituted, and wherein two R′ or R″ groups can together form a ring.

In Formula (15), preferably each R⁴ and R⁶ is independently selected from the group consisting of H, (hetero)alkyl, aryl, heterocycle, carbocycle, OR′, C(═O)R′, C(═O)NR′₂, C(═S)NR′₂, C(═NR′)NR′₂, NR′₂, preferably such that no sets consisting of adjacent atoms are present selected from the group consisting of —N—N—, —N—O—, —N—S—, —N—Si— and —N—P—, and wherein each R′ is independently selected from the group consisting of (hetero)alkyl, aryl, carbocycle, heterocycle, wherein all (hetero)alkyl, aryl, carbocycle, heterocycle moieties comprised in R⁴ and R⁶ can be unsubstituted or substituted, and wherein two R′ groups can together form a ring.

In Formula (15), preferably at least one X₅₀ is hydrogen, more preferably at least one X₅₀ is hydrogen and the other X₅₀ is hydrogen or C₁₋₃ alkyl, and most preferably both X₅₀ are hydrogen.

In Formula (15), optionally one or more of R¹, R², R³, R⁴, R⁵, R⁶, R₅₀ and S^{P}, is bound to a Construct B C^{B}, and/or S^{P}—C^{B}; wherein S^{P} is a spacer.

### Methods for Preparing a Compound of the Disclosure

The disclosure also relates to methods for preparing compounds of the disclosure, compositions of the disclosure, or intermediates of the disclosure.

One such method is a method for synthesizing
(i) a compound according to the disclosure, or a salt, solvate, hydrate, and/or an enantiomer thereof, or
(ii) a composition according to the disclosure;
   wherein said method comprises the step (PI), wherein step (PI) requires subjecting a compound Z or a salt, solvate, hydrate, and/or an enantiomer thereof, to photoisomerization, wherein compound Z comprises a (Z)-bicyclo[6.1.0]non-3-ene moiety, wherein at least one allylic carbon of said moiety is in the R-configuration and is substituted with R₄₈; R₄₈ is selected from the group consisting of —OH, —O-acetyl, —O—C₁₋₄ alkyl, halogen, active carbonate, and a releasable group;
the carbon atom at position 1 of said moiety is in the R-configuration;
the carbon atom at position 8 of said moiety is in the S-configuration. Preferably the carbon atom at position 9 of said moiety is substituted, more preferably with a group R₄₉ as defined herein. Preferably compound Z is an intermediate, or a salt, solvate, hydrate, and/or an enantiomer thereof, wherein the intermediate is according to Formula (INT15-3) or (INT13-3) as defined herein.

Due to the photoisomerization, compounds of the disclosure are formed, wherein R₄₈ may be in the axial or equatorial position (denoted by either “AX” or “EQ” in Formulae of the disclosure). If desired, these axial and equatorial isomers can be readily separated as shown in the Examples. This separation step is herein referred to as step (SEP), which preferably comprises applying chromatography, more preferably applying silica column chromatography and/or high-performance liquid chromatography (HPLC).

### Synthesis of 1,5-Derivatives

As used herein, “1,5-derivatives” refers to compounds of the disclosure of which the Formula contains “15”, such as Formula (IV-EQ15EN).

As used herein, step (15a) refers to a step of contacting 1,5-cyclooctadiene with a catalyst, preferably Rh₂(OAc)₄, ethyl diazoacetate, and an organic solvent, preferably dichloromethane. These starting materials are all commercially available. Step (15a) is preferably carried out at ambient temperatures, such as in a range of from 10 °C to 50 °C, preferably in a range of from 12 °C to 40 °C, more preferably in a range of from 15° to 30 °C. Preferably, the reaction mixture obtained in step (15a) is subjected to purification, preferably by chromatography, more preferably by silica column chromatography. Step (15a) affords a mixture of the endo and exo isomers of compound 1.1:

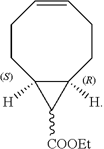

As use herein, step (15b) refers to a step of hydrolyzing the ester in the mixture of the endo and exo isomers of compound 1.1. Preferably, in step (15b) the mixture of compounds is dissolved in a solvent, preferably methanol, and contacted with an aqueous solution of a strong base, preferably NaOH or KOH. Step (15b) is preferably carried out at ambient temperature, such as in a range of from 10 °C to 50 °C, preferably in a range of from 12 °C to 40 °C, more preferably in a range of from 15° to 30 °C. Preferably, after the reaction is sufficiently complete, the solvent is removed by evaporation, and the residue is preferably subjected to extraction. Step (15b) yields a mixture of endo and exo isomers of compound 1.2:

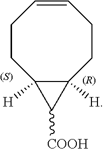

The disclosure relates to a method of synthesizing intermediate (INT15-1), wherein said method comprises step (15c). As used herein, step (15c) refers to a step of subjecting the mixture of endo and exo isomers of compound 1.2 to halolactonization, preferably iodolactonization. Preferably, in step (15c) the mixture of endo and exo isomers of compound 1.2 is contacted with an organic solvent, preferably dichloromethane, water, and a weak base, preferably hydrogencarbonate, and at least one halogen. Preferably, the at least one halogen is HA₂ and HA⁻, wherein HA is a halogen, more preferably the at least one halogen is I₂ and a iodide salt, preferably KI. Preferably, the reaction mixture obtained in step (15c) is subjected to extraction. Preferably, the reaction mixture obtained in step (15c) is subjected to purification, preferably by chromatography, more preferably by silica column chromatography. Step (15c) is preferably carried out at ambient temperatures, preferably in a range of from 10 °C to 50 °C, preferably in a range of from 12 °C to 40 °C, more preferably in a range of from 15° to 30 °C. Step (15c) affords intermediate (INT15-1) as defined herein.

The disclosure relates to a method of synthesizing intermediate (INT15-2), wherein said method comprises step (15d). As used herein, step (15d) refers to a step of subjecting the intermediate (INT15-1) to an elimination reaction. Preferably, in step (15d) intermediate (INT15-1) is contacted with an organic solvent, preferably toluene, and a strong base, preferably 1,8-diazabicyclo(5.4.0)undec-7-ene (DBU) or 1,5-diazabicyclo[4.3.0]non-5-ene (DBN). The reaction of step (15d) is preferably carried out at a temperature in a range of from 30 °C to 90 °C, preferably in a range of from 40 °C to 80 °C, more preferably in a range of from 45° to 65 °C. Preferably, the reaction mixture obtained in step (15d) is subjected to extraction. Preferably, the reaction mixture obtained in step (15d) is subjected to purification, preferably by chromatography, more preferably by silica column chromatography. Step (15d) yields intermediate (INT15-2).

The disclosure relates to a method of synthesizing intermediate (INT15-3), wherein said method comprises step (15e). As used herein, step (15e) refers to a step of subjecting the intermediate (INT15-2) to ester formation or hydrolysis. In step (15eA), the intermediate (INT15-2) is preferably contacted with water (for a hydrolysis reaction) or an alcohol (for ester formation), preferably methanol, and a strong base, preferably KOH and/or NaOH for a hydrolysis reaction, and preferably KOH, NaOH, and/or NaOCH₃ for ester formation. The reaction of step (15e) is preferably carried out at a temperature in a range of from 30 °C to 90 °C, preferably in a range of from 40 °C to 80 °C, more preferably in a range of from 450 to 65 °C. Step (15e) affords intermediate (INT15-3), wherein preferably IN² is —O—CH₃, and preferably IN³ is hydrogen, which can be obtained in an ester formation reaction using methanol.

### Synthesis of 1,3-Derivatives

As used herein, “1,3-derivatives” refers to compounds of the disclosure of which the Formula contains “13”, such as Formula (IV-EQ13EN).

As used herein, step (13a) refers to a step of contacting 1,3-cyclooctadiene with a catalyst, preferably Rh₂(OAc)₄, ethyl diazoacetate, and an organic solvent, preferably dichloromethane. These starting materials are all commercially available. Step (13a) is preferably carried out at ambient temperatures, such as in a range of from 10 °C to 50 °C, preferably in a range of from 12 °C to 40 °C, more preferably in a range of from 15° to 30 °C. Preferably, the reaction mixture obtained in step (13a) is subjected to purification, preferably by chromatography, more preferably by silica column chromatography. Step (13a) affords a mixture of compounds 1.11:

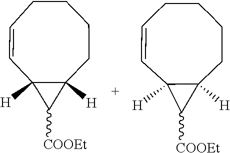

As used herein, step (13b) refers to a step of hydrolyzing the ester in the mixture of compounds 1.11. Preferably, in step (13b) the mixture of compounds is dissolved in a solvent, preferably methanol, and contacted with an aqueous solution of a strong base, preferably NaOH or KOH. Step (13b) is preferably carried out at ambient temperatures, such as in a range of from 10 °C to 50 °C, preferably in a range of from 12 °C to 40 °C, more preferably in a range of from 150 to 30 °C. Preferably, after the reaction is sufficiently complete, the solvent is removed by evaporation, and the residue is preferably subjected to extraction. Step (13b) yields a mixture of diastereoisomers of compound 1.12:

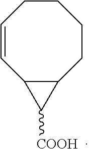 [SMILES: O=C(O)C1C2/C=C\CCCCC21]

The disclosure relates to a method of synthesizing intermediate (INT13-1), wherein said method comprises step (13c). As used herein, step (13c) refers to a step of subjecting the mixture of diastereoisomers of compound 1.12 to halolactonization, preferably iodolactonization. Preferably, in step (13c) the mixture of diastereoisomers of compound 1.12 is contacted with an organic solvent, preferably dichloromethane, water, and a weak base, preferably hydrogencarbonate, and at least one halogen. Preferably, the at least one halogen is HA₂ and HA⁻, wherein HA is a halogen, more preferably the at least one halogen is I₂ and a iodide salt, preferably KI. Preferably, the reaction mixture obtained in step (13c) is subjected to extraction. Preferably, the reaction mixture obtained in step (13c) is subjected to purification, preferably by chromatography, more preferably by silica column chromatography. Step (13c) is preferably carried out at ambient temperatures, preferably in a range of from 10 °C to 50 °C, preferably in a range of from 12 °C to 40 °C, more preferably in a range of from 15° to 30 °C. Step (13c) affords intermediate (INT13-1) as defined herein.

The disclosure relates to a method of synthesizing intermediate (INT13-2), wherein said method comprises step (13d). As used herein, step (13d) refers to a step of subjecting the intermediate (INT13-1) to an elimination reaction. Preferably, in step (13d) intermediate (INT13-1) is contacted with an organic solvent, preferably toluene, and a strong base, preferably 1,8-diazabicyclo(5.4.0)undec-7-ene (DBU) or 1,5-diazabicyclo[4.3.0]non-5-ene (DBN). The reaction of step (13d) is preferably carried out at a temperature in a range of from 30 °C to 90 °C, preferably in a range of from 40 °C to 80 °C, more preferably in a range of from 450 to 65 °C. Preferably, the reaction mixture obtained in step (13d) is subjected to extraction. Preferably, the reaction mixture obtained in step (13d) is subjected to purification, preferably by chromatography, more preferably by silica column chromatography. Step (13d) yields intermediate (INT13-2)

The disclosure relates to a method of synthesizing intermediate (INT13-3), wherein said method comprises step (13e). As used herein, step (13e) refers to a step of subjecting the intermediate (INT13-2) to ester formation or hydrolysis (step (13eA)) and acetylation (step (13eB)). Since steps (13eA) and (13eB) can be carried out sequentially without the requirement of a purification step, these steps are collectively referred to as step (13e). In step (13eA), the intermediate (INT13-2) is preferably contacted with water (for hydrolysis) or an alcohol (for ester formation), preferably methanol, and a strong base, preferably KOH and/or NaOH for a hydrolysis reaction, and preferably KOH, NaOH, and/or NaOCH₃ for ester formation. The reaction of step (13eA) is preferably carried out at a temperature in a range of from 30 °C to 90 °C, preferably in a range of from 40 °C to 80 °C, more preferably in a range of from 450 to 65 °C. After the reaction is sufficiently complete, the reaction mixture of step (13eA) is preferably subjected to evaporation to remove the solvent, and the residue is redissolved in an organic solvent, preferably dimethylformamide (DMF), to form a second solution. Then, in step (13eB) said second solution is preferably contacted with iodomethane, a base, preferably pyridine and/or 4-dimethylaminopyridine (DMAP), an organic solvent, preferably toluene, and an acetylating agent, preferably acetic anhydride. Preferably, in step (13eB) the contacting is at a temperature in a range of from −20° to 10 °C, preferably in a range of from −5 °C to 5°, most preferably of about 0 °C, after which the reaction mixture is allowed to reach ambient temperature, preferably in a range of from 10 °C to 50 °C, more preferably in a range of from 12 °C to 40 °C, most preferably in a range of from 15° to 30 °C. Preferably, then the reaction mixture of step (13eB) is quenched, and preferably extracted, and preferably subjected to purification, preferably by applying chromatography, more preferably by applying silica column chromatography. Step (13e) affords intermediate (INT13-3), wherein preferably IN² is —O—CH₃, and preferably IN⁴ is acetyl, which can be obtained with an ester formation in step (13eA) using methanol, and the acetylation of step (13eB).

### Synthesis of Conjugates

The disclosure also relates to a method of synthesizing a compound of the disclosure wherein R₄₈ is a releasable group, wherein said method comprises step (CP), wherein step (CP) requires coupling S′ or —(S^{P})ⱼ—C^{A} as defined herein to a compound of the disclosure wherein R₄₈ is selected from the group consisting of —OH, halogen, and active carbonate. If a group S^{P} is coupled in said method, said step is followed by a step of coupling C^{A} as defined herein to said group S^{P}. Coupling strategies are well-known in the art and readily available to the skilled person.

Preferably, in said method of synthesizing a compound of the disclosure wherein R₄₈ is a releasable group, wherein said method comprises the step of contacting —(S^{P})ⱼ—C^{A} with a compound of the disclosure wherein R₄₉ is an active carbonate, preferably wherein R₄₈ is —OC(O)O—N-succinimidyl, and preferably j is 0. Since the group —(S^{P})ⱼ—C^{A} will contain at least one atom selected from the group consisting of O, S, secondary N, and tertiary N; said group —(S^{P})ⱼ—C^{A} will be coupled via said at least one atom to the —O—C(O)O— group, since N-succinimidyl is a good leaving group. Thus, a compound according to the disclosure wherein R⁴⁸ is a releasable group can be formed. Interestingly and advantageously, in this method, using a compound of the disclosure wherein R₄₈ is an active carbonate, the nature of R₄₉ does not matter. Even if R₄₉ is an active ester, reactions with the group —(S^{P})ⱼ—C^{A} are typically selective for the active carbonate at the R₄₈ position.

Synthetic Routes Towards Intermediates of the Disclosure and/or Compounds of the Disclosure

Preferably, the method for synthesizing an intermediate of Formula (INT15-1) or the enantiomer thereof comprises the step (15c) as defined herein. Preferably, the method for synthesizing an intermediate of Formula (INT15-1) or the enantiomer thereof comprises the step (15b) as defined herein, followed by step (15c) as defined herein. Preferably, the method for synthesizing an intermediate of Formula (INT15-1) or the enantiomer thereof comprises the step (15a) as defined herein, followed by the step (15b) as defined herein, followed by step (15c) as defined herein.

Preferably, the method for synthesizing an intermediate of Formula (INT15-2) or the enantiomer thereof comprises the step (15d) as defined herein. Preferably, the method for synthesizing an intermediate of Formula (INT15-2) or the enantiomer thereof comprises the step (15c) as defined herein, followed by step (15d) as defined herein. Preferably, the method for synthesizing an intermediate of Formula (INT15-2) or the enantiomer thereof comprises the step (15b) as defined herein, followed by the step (15c) as defined herein, followed by step (15d) as defined herein. Preferably, the method for synthesizing an intermediate of Formula (INT15-2) or the enantiomer thereof comprises the step (15a) as defined herein, followed by step (15b) as defined herein, followed by the step (15c) as defined herein, followed by step (15d) as defined herein.

Preferably, the method for synthesizing an intermediate of Formula (INT15-3) or the enantiomer thereof comprises the step (15e) as defined herein. Preferably, the method for synthesizing an intermediate of Formula (INT15-3) or the enantiomer thereof comprises the step (15d) as defined herein, followed by step (15e) as defined herein. Preferably, the method for synthesizing an intermediate of Formula (INT15-3) or the enantiomer thereof comprises the step (15c) as defined herein, followed by the step (15d) as defined herein, followed by step (15e) as defined herein. Preferably, the method for synthesizing an intermediate of Formula (INT15-3) or the enantiomer thereof comprises the step (15b) as defined herein, followed by step (15c) as defined herein, followed by the step (15d) as defined herein, followed by step (15e) as defined herein. Preferably, the method for synthesizing an intermediate of Formula (INT15-3) or the enantiomer thereof comprises the step (15a) as defined herein, followed by step (15b) as defined herein, followed by step (15c) as defined herein, followed by the step (15d) as defined herein, followed by step (15e) as defined herein.

Preferably, the method for synthesizing an intermediate of Formula (INT13-1) or the enantiomer thereof comprises the step (13c) as defined herein. Preferably, the method for synthesizing an intermediate of Formula (INT13-1) or the enantiomer thereof comprises the step (13b) as defined herein, followed by step (13c) as defined herein. Preferably, the method for synthesizing an intermediate of Formula (INT13-1) or the enantiomer thereof comprises the step (13a) as defined herein, followed by the step (13b) as defined herein, followed by step (13c) as defined herein.

Preferably, the method for synthesizing an intermediate of Formula (INT13-2) or the enantiomer thereof comprises the step (13d) as defined herein. Preferably, the method for synthesizing an intermediate of Formula (INT13-2) or the enantiomer thereof comprises the step (13c) as defined herein, followed by step (13d) as defined herein. Preferably, the method for synthesizing an intermediate of Formula (INT13-2) or the enantiomer thereof comprises the step (13b) as defined herein, followed by the step (13c) as defined herein, followed by step (13d) as defined herein. Preferably, the method for synthesizing an intermediate of Formula (INT13-2) or the enantiomer thereof comprises the step (13a) as defined herein, followed by step (13b) as defined herein, followed by the step (13c) as defined herein, followed by step (13d) as defined herein.

Preferably, the method for synthesizing an intermediate of Formula (INT13-3) or the enantiomer thereof comprises the step (13e) as defined herein. Preferably, the method for synthesizing an intermediate of Formula (INT13-3) or the enantiomer thereof comprises the step (13d) as defined herein, followed by step (13e) as defined herein. Preferably, the method for synthesizing an intermediate of Formula (INT13-3) or the enantiomer thereof comprises the step (13c) as defined herein, followed by the step (13d) as defined herein, followed by step (13e) as defined herein. Preferably, the method for synthesizing an intermediate of Formula (INT13-3) or the enantiomer thereof comprises the step (13b) as defined herein, followed by step (13c) as defined herein, followed by the step (13d) as defined herein, followed by step (13e) as defined herein. Preferably, the method for synthesizing an intermediate of Formula (INT13-3) or the enantiomer thereof comprises the step (13a) as defined herein, followed by step (13b) as defined herein, followed by step (13c) as defined herein, followed by the step (13d) as defined herein, followed by step (13e) as defined herein.

Preferably, the method for synthesizing a compound of the disclosure comprises step (PI) as defined herein followed by step (SEP) as defined herein.

If the compound of the disclosure to be synthesized has a group R₄₈ that is a releasable group the method for synthesizing a compound of the disclosure may comprise (i) step (PI) as defined herein; (ii) steps (PI) and (SEP) as defined herein; (iii) steps (PI) and (CP) as defined herein; or (iv) steps (PI), (SEP), and (CP) as defined herein.

Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15e) as defined herein, followed by step (PI) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15d) as defined herein, followed by step (15e) as defined herein, followed by step (PI) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15c) as defined herein, followed by the step (15d) as defined herein, followed by step (15e) as defined herein, followed by step (PI) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15b) as defined herein, followed by step (15c) as defined herein, followed by the step (15d) as defined herein, followed by step (15e) as defined herein, followed by step (PI) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15a) as defined herein, followed by step (15b) as defined herein, followed by step (15c) as defined herein, followed by the step (15d) as defined herein, followed by step (15e) as defined herein, followed by step (PI) as defined herein.

Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises step (SEP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (PI) as defined herein, followed by step (SEP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15e) as defined herein, followed by step (PI) as defined herein, followed by step (SEP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15d) as defined herein, followed by step (15e) as defined herein, followed by step (PI) as defined herein, followed by step (SEP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15c) as defined herein, followed by the step (15d) as defined herein, followed by step (15e) as defined herein, followed by step (PI) as defined herein, followed by step (SEP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15b) as defined herein, followed by step (15c) as defined herein, followed by the step (15d) as defined herein, followed by step (15e) as defined herein, followed by step (PI) as defined herein, followed by step (SEP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15a) as defined herein, followed by step (15b) as defined herein, followed by step (15c) as defined herein, followed by the step (15d) as defined herein, followed by step (15e) as defined herein, followed by step (PI) as defined herein, followed by step (SEP) as defined herein.

Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises step (SEP) as defined herein, followed by step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises step (PI) as defined herein, followed by step (SEP) as defined herein, followed by step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15e) as defined herein, followed by step (PI) as defined herein, followed by step (SEP) as defined herein, followed by step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15d) as defined herein, followed by step (15e) as defined herein, followed by step (PI) as defined herein, followed by step (SEP) as defined herein, followed by step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15c) as defined herein, followed by the step (15d) as defined herein, followed by step (15e) as defined herein, followed by step (PI) as defined herein, followed by step (SEP) as defined herein, followed by step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15b) as defined herein, followed by step (15c) as defined herein, followed by the step (15d) as defined herein, followed by step (15e) as defined herein, followed by step (PI) as defined herein, followed by step (SEP) as defined herein, followed by step (CP) as defined herein. Preferably, the method for synthesizing a compound according to any one of Formulae (II-EQ15), (II-AX15), (III-EQ15), (III-AX15), (IV-EQ15EN), (IV-AX15EN), (IV-EQ15EX), (IV-AX15EX), (V-EQ15EN), (V-AX15EN), (V-EQ15EX), and (V-AX15EX) or the enantiomer thereof comprises the step (15a) as defined herein, followed by step (15b) as defined herein, followed by step (15c) as defined herein, followed by the step (15d) as defined herein, followed by step (15e) as defined herein, followed by step (PI) as defined herein, followed by step (SEP) as defined herein, followed by step (CP) as defined herein.

Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13e) as defined herein, followed by step (PI) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13d) as defined herein, followed by step (13e) as defined herein, followed by step (PI) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13c) as defined herein, followed by the step (13d) as defined herein, followed by step (13e) as defined herein, followed by step (PI) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13b) as defined herein, followed by step (13c) as defined herein, followed by the step (13d) as defined herein, followed by step (13e) as defined herein, followed by step (PI) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13a) as defined herein, followed by step (13b) as defined herein, followed by step (13c) as defined herein, followed by the step (13d) as defined herein, followed by step (13e) as defined herein, followed by step (PI) as defined herein.

Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises step (SEP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (PI) as defined herein, followed by step (SEP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13e) as defined herein, followed by step (PI) as defined herein, followed by step (SEP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13d) as defined herein, followed by step (13e) as defined herein, followed by step (PI) as defined herein, followed by step (SEP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13c) as defined herein, followed by the step (13d) as defined herein, followed by step (13e) as defined herein, followed by step (PI) as defined herein, followed by step (SEP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13b) as defined herein, followed by step (13c) as defined herein, followed by the step (13d) as defined herein, followed by step (13e) as defined herein, followed by step (PI) as defined herein, followed by step (SEP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13a) as defined herein, followed by step (13b) as defined herein, followed by step (13c) as defined herein, followed by the step (13d) as defined herein, followed by step (13e) as defined herein, followed by step (PI) as defined herein, followed by step (SEP) as defined herein.

Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises step (SEP) as defined herein, followed by step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises step (PI) as defined herein, followed by step (SEP) as defined herein, followed by step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13e) as defined herein, followed by step (PI) as defined herein, followed by step (SEP) as defined herein, followed by step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13d) as defined herein, followed by step (13e) as defined herein, followed by step (PI) as defined herein, followed by step (SEP) as defined herein, followed by step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13c) as defined herein, followed by the step (13d) as defined herein, followed by step (13e) as defined herein, followed by step (PI) as defined herein, followed by step (SEP) as defined herein, followed by step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13b) as defined herein, followed by step (13c) as defined herein, followed by the step (13d) as defined herein, followed by step (13e) as defined herein, followed by step (PI) as defined herein, followed by step (SEP) as defined herein, followed by step (CP) as defined herein. Preferably, the method for synthesizing a compound according to any one of Formulae (II-EQ13), (II-AX13), (III-EQ13), (III-AX13), (IV-EQ13EN), (IV-AX13EN), (IV-EQ13EX), (IV-AX13EX), (V-EQ13EN), (V-AX13EN), (V-EQ13EX), and (V-AX13EX) or the enantiomer thereof comprises the step (13a) as defined herein, followed by step (13b) as defined herein, followed by step (13c) as defined herein, followed by the step (13d) as defined herein, followed by step (13e) as defined herein, followed by step (PI) as defined herein, followed by step (SEP) as defined herein, followed by step (CP) as defined herein.

### Alternative Synthesis Route to 1,3-Derivatives

The disclosure also relates to a method for synthesizing compounds 1.17a and 1.17b as disclosed herein in Example 1.14, wherein said method comprises step (17a). In step (17a), an intermediate of Formula (INT13-2) or an enantiomer thereof is subjected to photoisomerization, yielding compounds 1.17a and 1.17b as disclosed herein in Example 1.14 or enantiomers thereof. Said compound 1.17a is herein also referred to as an intermediate of Formula (INTAX13-4), and said compound 1.17b is herein also referred to as an intermediate of Formula (INTEQ13-4). Preferably, after photoisomerization a separation step is carried out, preferably using chromatography, to separate said compounds 1.17a and 1.17b.

The disclosure also relates to a method for synthesizing compound 1.18a or another salt thereof as disclosed herein in Example 1.15, wherein said method comprises step (17b-1). As used herein, step (17b-1) refers to a step of subjecting compound 1.17a to hydrolysis. In step (17b-1), the compound 1.17a is preferably contacted with a solvent, preferably methanol, and a strong base, preferably KOH and/or NaOH. Preferably, the reaction of step (17b-1) is carried out at a temperature in a range of from 20 °C to 70°, preferably in a range of from 25 °C to 60 °C, and most preferably of from 30 °C to 50 °C. Once the reaction of step (17b-1) is sufficiently complete, it is preferably quenched, preferably by adding a solvent, preferably ethyl acetate. Thereafter, the solvent is preferably removed, preferably by evaporation, resulting in a residue. The residue is preferably contacted with a solvent, preferably acetonitrile, resulting in a mixture from which compound 1.18a or another salt thereof is isolated, preferably via filtration. Step (17b-1) affords compound 1.18a or another salt thereof.

The disclosure also relates to a method for synthesizing compound 1.19a as disclosed herein in Example 1.17, wherein said method comprises step (17c-1). As used herein, step (17c-1) refers to a step of subjecting compound 1.18a to ester formation. Preferably, in step (17c-1) compound 1.18a is contacted with a solvent, preferably an alcohol, more preferably methanol, and a methoxide salt, preferably sodium methoxide. The resulting solution is preferably mixed at a temperature in a range of from 20 °C to 80°, preferably in a range of from 30 °C to 70 °C, and most preferably of from 40 °C to 60 °C. Once the reaction of step (17c-1) is sufficiently complete, the reaction is preferably neutralized, preferably by contacting the reaction mixture with an acidic ion-exchange resin. Thereafter, the resulting mixture is preferably purified, preferably by filtration. Step (17c-1) affords compound 1.19a.

The disclosure also pertains to a method for synthesizing a compound of the disclosure having a structure of Formula (III-AX13) or (V-AX13EN) or the enantiomers thereof, wherein said method comprises step (17d-1). As used herein, step (17d-1) refers to a step of subjecting compound 1.19a or the enantiomer thereof to activation. Preferably, in step (17d-1) compound 1.19a or the enantiomer thereof is contacted with a solvent, preferably dimethylsulfoxide, and an activating agent, preferably a carbonate, more preferably di-(N-succinimide)-carbonate. Preferably, the reaction in step (17d-1) is carried out at ambient temperature, preferably in a range of from 10 °C to 50 °C, preferably in a range of from 12 °C to 40 °C, more preferably in a range of from 15° to 30 °C. Step(17d-1) affords a compound of the disclosure having a structure of Formula (III-AX13) or (V-AX13EN) or the enantiomers thereof, wherein in said compounds R₄₈ is an active carbonate, preferably —OC(O)—O—N— succinimidyl, and R₄₉ is an active ester, preferably —C(O)—O—N-succinimidyl.

The disclosure also relates to a method for synthesizing compound 1.18b or another salt thereof as disclosed herein in Example 1.16, wherein said method comprises step (17b-2). As used herein, step (17b-2) refers to a step of subjecting compound 1.17b to hydrolysis. In step (17b-2), the compound 1.17b is preferably contacted with a solvent, preferably methanol, and a strong base, preferably KOH and/or NaOH. Preferably, the reaction of step (17b-2) is carried out at a temperature in a range of from 20 °C to 70°, preferably in a range of from 25 °C to 60 °C, and most preferably of from 30 °C to 50 °C. Once the reaction of step (17b-2) is sufficiently complete, it is preferably quenched, preferably by adding a solvent, preferably ethyl acetate. Thereafter, the solvent is preferably removed, preferably by evaporation, resulting in a residue. The residue is preferably contacted with a solvent, preferably acetonitrile, resulting in a mixture from which compound 1.18b or another salt thereof is isolated, preferably via filtration. Step (17b-2) affords compound 1.18b or another salt thereof.

The disclosure also relates to a method for synthesizing compound 1.19b:

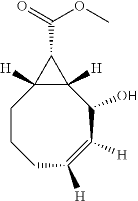

and/or the enantiomer thereof, wherein said method comprises step (17c-2). As used herein, step (17c-2) refers to a step of subjecting compound 1.18b and/or the enantiomer thereof to ester formation. Preferably, in step (17c-2) compound 1.18b and/or the enantiomer thereof is contacted with a solvent, preferably an alcohol, more preferably methanol, and a methoxide salt, preferably sodium methoxide. The resulting solution is preferably mixed at a temperature in a range of from 20 °C to 80°, preferably in a range of from 30 °C to 70 °C, and most preferably of from 40 °C to 60 °C. Once the reaction of step (17c-2) is sufficiently complete, the reaction is preferably neutralized, preferably by contacting the reaction mixture with an acidic ion-exchange resin. Thereafter, the resulting mixture is preferably purified, preferably by filtration. Step (17c-2) affords compound 1.19b and/or the enantiomer thereof.

The disclosure also pertains to a method for synthesizing a compound of the disclosure having a structure of Formula (III-EQ13) or (V-EQ13EN) or the enantiomers thereof, wherein said method comprises step (17d-2). As used herein, step (17d-2) refers to a step of subjecting compound 1.19b or the enantiomer thereof to activation. Preferably, in step (17d-2) compound 1.19b or the enantiomer thereof is contacted with a solvent, preferably dimethylsulfoxide, and an activating agent, preferably a carbonate, more preferably di-(N-succinimide)-carbonate. Preferably, the reaction in step (17d-2) is carried out at ambient temperature, preferably in a range of from 10 °C to 50 °C, preferably in a range of from 12 °C to 40 °C, more preferably in a range of from 15° to 30 °C. Step(17d-1) affords a compound of the disclosure having a structure of Formula (III-EQ13) or (V-EQ13EN) or the enantiomers thereof, wherein in said compounds R₄₈ is an active carbonate, preferably —OC(O)—O—N— succinimidyl, and R₄₉ is an active ester, preferably —C(O)—O—N-succinimidyl.

### Synthesis of Exo Compounds

The disclosure also relates to a method of synthesizing compounds of the disclosure wherein the R₄₉ group is in the exo position, wherein said method comprises step (EX). As used herein, step (EX) refers to a step of contacting a compound of Formula (IV-EQ13EN), (IV-AX13EN), (V-EQ13EN), (V-AX13EN), (IV-EQ15EN), (IV-AX15EN), (V-EQ15EN), (V-AX15EN), or an enantiomer thereof, with a strong base, preferably tert-butoxide, and an organic solvent. The organic solvent is typically selected from the group consisting of tetrahydrofuran, ethanol, ether, and combinations thereof. A preferred combination is tetrahydryofuran and ethanol. Preferably, the ethanol is dry ethanol. Preferably, the ether is wet ether. Preferably, the organic solvent in step (EX) is tetrahydrofuran or ethanol

Preferably, the reaction in step (EX) is carried out at ambient temperature, preferably in a range of from 10 °C to 50 °C, preferably in a range of from 12 °C to 40 °C, more preferably in a range of from 15° to 30 °C. Once the reaction is sufficiently complete, preferably the solvent is removed, preferably by evaporation, yielding a residue. Preferably, the residue is purified, preferably using chromatography, more preferably using silica column chromatography. Depending on the starting material, step (EX) yields a compound of Formula (IV-EQ13EX), (IV-AX13EX), (V-EQ13EX), (V-AX13EX), (IV-EQ15EX), (IV-AX15EX), (V-EQ15EX), (V-AX15EX), or an enantiomer thereof.

### Medical Use

The disclosure further pertains to a compound, composition, or combination, of the disclosure for use as a medicament. As is known to the skilled person, the use as a medicament means that the dienophile or the combination is used in the treatment of a subject. Preferably, said subject is a human being.

The disclosure also pertains to the dienophile of the disclosure or the combination according to the disclosure for use in the treatment of a disease in a subject.

In principle, since virtually any drug can be connected to the (E)-bicyclo[6.1.0]non-3-ene moiety of the compound of the disclosure, any disease for which drugs are available can be treated using a compound of the disclosure. Still, in relation to the disclosure and the disclosure as a whole, the disease is preferably selected from the group consisting of cancer, central nervous system (CNS) diseases, infection, inflammation, and cardiovascular diseases. Preferably, the disease is cancer.

In relation to the disclosure and the disclosure as a whole, the subject is preferably a human.

The disclosure also relates to a method of treating a disease in a subject, wherein said method comprises administering to said subject a compound of the disclosure, a composition of the disclosure, or a combination of the disclosure.

The disclosure also relates to a use of of a compound of the disclosure, a composition of the disclosure, or a combination of the disclosure, for the manufacture of a medicament for the treatment of a disease.

When administering the Prodrug, i.e. a compound of the disclosure wherein C^{A} is a drug, and the Activator to a living system, such as an animal or human, preferably the Activator (preferably connected to a Targeting Agent) is administered first, and it will take a certain time period before the Activator has reached the Primary Target. This time period may differ from one application to the other and may be minutes, days or weeks. After the time period of choice has elapsed, the Prodrug is administered, which will find and react with the Activator. The Activator will thus activate the Prodrug and/or afford Drug release at the Primary Target. Preferably, the time interval between the administration of the Activator and the Prodrug is between 10 minutes and 4 weeks. Preferably, the time interval between the administration of the Activator and the Prodrug is between 1 hour and 2 weeks, preferably between 1 and 168 hours, more preferably between 1 and 120 hours, even more preferably between 1 and 96 hours, most preferably between 3 and 72 hours.

The compounds, compositions, and combinations of the disclosure can be administered via different routes including but not limited to intravenous or subcutaneous injection, intraperitoneal, local injection, oral administration, rectal administration and inhalation. Formulations suitable for these different types of administrations are known to the skilled person. Prodrugs or Activators according to the disclosure can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include for example saline, buffered saline, dextrose, water, glycerol, ethanol, and combinations thereof. The formulation should suit the mode of administration.

It will be understood that the chemical entities administered, viz. the Prodrug and the Activator, can be in a modified form that does not alter the chemical functionality of said chemical entity, such as salts, hydrates, or solvates thereof.

After administration of the Activator, and before the administration of the Prodrug, it is preferred to remove excess Activator by means of a Clearing Agent in cases when Activator activation in circulation is undesired and when natural Activator clearance is insufficient. A Clearing Agent is an agent, compound, or moiety that is administered to a subject for the purpose of binding to, or complexing with, an administered agent (in this case the Activator) of which excess is to be removed from circulation. The Clearing Agent is capable of being directed to removal from circulation. The latter is generally achieved through liver receptor-based mechanisms, although other ways of secretion from circulation exist, as are known to the skilled person. In the disclosure, the Clearing Agent for removing circulating Activator, preferably comprises a dienophile moiety, e.g. as discussed herein, capable of reacting to the diene moiety of the Prodrug.

In other preferred embodiments the Prodrug is administered first, followed by the Activator, wherein the time interval between the administration of the two components ranges from 1 minute to 12 weeks, preferably 1 minute to 2 weeks, preferably from 10 minutes to 3 days. In some embodiments, the Prodrug and Activator are administered at the same time, either as two separate administrations or as a co-administration.

The compound of the disclosure can include a masking moiety that optionally also provides additional advantageous properties. For example, the compound of the disclosure can contain a masking moiety that simultaneously extends the serum half-life and/or targets the compound of the disclosure to a desired site. Preferably, this element is removed chemically, by administration of an activator compound, at the desired body location (e.g., in the tumor microenvironment), restoring pharmacokinetic properties to the payload molecule (e.g., IL12) substantially similar to the naturally occurring payload molecule.

The compound of the disclosures may be targeted to a desired cell or tissue in an active or passive way. As described herein active targeting is typically accomplished through the action of a targeting agent, e.g. a small molecule, peptide, aptamer, protein, antibody, or antibody fragment, binding a target expressed on the target location (for example a tumor-specific antigen or receptor). Passive targeting on the other hand relies on the passive accumulation of the compound of the disclosure at the desired cell or tissue by modulating pharmacokinetics and distribution via molecular properties of the compound of the disclosure (e.g. size). Typically, in these situations the compound of the disclosure contains one or more moieties that extends and modulates the half-life of the construct (e.g. PEG, albumin binders). Preferably the compound of the disclosure is designed to passively target the tumor by means of the Enhanced Permeability and Retention (EPR) effect.

The targeting agent may be attached to the the (E)-bicyclo[6.1.0]non-3-ene moiety of the compound of the disclosure via a chemically cleavable or non-cleavable linker. If attached by a non-cleavable linker, the targeting domain may further aid in retaining the compound of the disclosure at the target site, for example a tumor, and may be considered a retention domain. The targeting domain does not necessarily need to be directly linked to the payload, and may be linked via another element of the compound of the disclosure.

In one aspect the disclosure provides a compound of the disclosure comprising a masking moiety, e.g. a polyethylene glycol (PEG) masking domain, preferably as R₄₉. The masking moiety is conjugated to the the (E)-bicyclo[6.1.0]non-3-ene moiety of the compound of the disclosure, through a linker, and can be separated from the payload by cleavage of the construct due to reaction of the Trigger with a separately provided activator.

Preferably, the Prodrug of the disclosure is designed to be targeted to the site of desired drug activity, for example the tumor microenvironment, while the presence of the compound of the disclosure at non-desired locations (e.g. non-tumor tissue, or blood circulation), is decreasing over time. Administering the activator separated in time from the compound of the disclosure, offers selective drug activity concentrated at the desired locations, while avoiding off-target activity and reducing overall toxicity of drug therapy.

In one embodiment, the compound of the disclosure is provided before the activator. Herein, the activator is provided only once the compound of the disclosure is present at the desired location and at the desired local concentration, while at the same time the compound of the disclosure has been cleared from circulation and non-target tissues to a desired reduced level as compared to the initial level occurring directly after the administration.

In another embodiment, the activator is administered to the subject before the compound of the disclosure is administered to said subject. Herein, the activator may typically be prelocalized at the target site, for example the tumor microenvironment. Such prelocalization can be achieved by injecting a construct comprising one or more activator moieties at or in the vicinity of the target site. Such construct can comprise a biomolecule or a polymer, such as hyaluronic acid, to which the one or more activator moieties are covalently linked. The construct typically has a size that enables it to remain at or in the vicinity of the target site, and it is not cleared efficiently. Prelocalization of the activator can also be achieved by conjugation of the activator to a targeting agent such as a small molecule, peptide, aptamer, protein, antibody, or antibody fragment, binding a target expressed on the target location (for example a tumor-specific antigen). This targeting agent will provide the activator with active tissue targeting. In conjunction, a compound of the disclosure of the disclosure may next be injected only once the targeted activator is present at the desired location and at the desired local concentration. If a targeted activator is used, the compound of the disclosure is preferably administered when the activator has been cleared from circulation and non-target tissues to a desired reduced level as compared to the initial level directly after administration of the activator. As described herein the prelocalized activator will activate the attenuated Prodrug of the disclosure, thereby restoring complete or nearly complete activity of the drug moiety present in the Prodrug of the disclosure. In other embodiments the activator is targeted to the target tissue in a passive way, as described herein for the compound of the disclosure, for example by comprising one or more activator moieties in a construct that targets the tumor by means of the EPR effect (e.g. diene-containing nanoparticles, diene polymer conjugates). In other embodiments both the compound of the disclosure and the activator are targeted to the target tissue by either passive or active targeting as described herein.

The compound of the disclosure can further include a targeting agent such as a small molecule, peptide, antibody, or antibody fragment, binding a target expressed on the target location (for example a tumor-specific antigen). This targeting agent will provide the compound of the disclosure with active tissue targeting. As described herein, when the compound of the disclosure includes a targeting agent, preferably the activator is provided separately only once the distribution of the compound of the disclosure throughout the body is favourable.

In a different embodiment, the activator is conjugated to a targeting agent, comprising a small molecule, (oligo)nucleotide, aptamer, carbohydrate, protein, peptide, peptoid, antibody, or antibody fragment, binding a target expressed on the target location (for example a tumor-specific antigen). The diene, preferably a tetrazine, and the targeting agent may be joined directly, or through a non-cleavable linker.

The half-life extension element of the compound of the disclosure can be, for example, human serum albumin, an antigen-binding polypeptide or small molecule that binds human serum albumin, an immunoglobulin Fc, or a water-soluble polypeptide such as polysarcosine, or an optionally branched or multi-armed polyethylene glycol (PEG), all previously employed in the art to extend serum half-life.

In some embodiments, the masking moiety can also function as a serum half-life extension element (e.g. polyethylene glycol). In some other embodiments, the compound of the disclosure comprises a separate serum half-life extension element.

Preferably, the masking agent also functions as the targeting agent.

In addition to serum half-life extension and/or masking moieties, the pharmaceutical compositions described herein preferably comprise one or more targeting agents that bind to one or more target antigens or one or more regions on a single target antigen. It is contemplated herein that a compound of the disclosure is cleaved, for example, after the compound of the disclosure has bound to a target antigen present in a disease-specific microenvironment. At least one target antigen is involved in and/or associated with a disease, disorder or condition. Exemplary target antigens include those associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease.

In some embodiments, the target antigen is a cell surface molecule such as a protein, lipid or polysaccharide. In some embodiments, the target antigen is present on a tumor cell, virally infected cell, bacterially infected cell, damaged red blood cell, arterial plaque cell, or fibrotic tissue cell. Cell surface target antigens typically are expressed on the surface of a diseased cell or tissue, for example a tumor or a cancer cell, or an immune cell. Such target antigens for tumors include but are not limited to Trophoblast glycoprotein (5T4), Tumor-associated calcium signal transducer 2 (Trop2), CGS-2, EpCAM, EGFR, HER-2, HER-3, c-Met, FOLR1, TAG72, and CEA.

In other embodiments, the target antigen is a molecule such as a protein, lipid or polysaccharide present in the direct vicinity of the diseased cell, e.g. the tumor microenvironment. Typically these molecules are found in the extracellular matrix, and not anymore directly connected to a cell. Such target antigens for tumors include but are not limited to Fibroblast activation protein alpha (FAPa), Tenacin C, Tenacin W, Fibronectin EDB (EDB-FN), and fibronectin EIIIB domain.

In some embodiments, the targeting polypeptides independently comprise a scFv, a VH domain, a VL domain, a non-Ig domain, or a ligand that specifically binds to the target antigen. In some embodiments, the targeting polypeptide serves as a retention domain and is attached to the drug via a non-cleavable linker.

This disclosure also relates to pharmaceutical compositions that comprise a compound of the disclosure or a composition according to the disclosure. Typically, the pharmaceutical composition contains one or more physiologically acceptable carriers and/or excipients.

The disclosure also relates to therapeutic methods that include administering to a subject in need thereof an effective amount of a compound of the disclosure, and/or a pharmaceutical composition thereof, and administering (either before, after or simultaneously with the administration of the compound of the disclosure) an effective amount of the activator molecule. Typically, the subject has, or is at risk of developing, a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease.

The disclosure further relates to methods for treating a tumor or cancer that include administering to a subject in need thereof an effective amount of a compound of the disclosure, and administering (either before, after or simultaneously with the administration of the compound of the disclosure) an effective amount of the activator molecule. In some embodiments, the method for treating a tumor or cancer can include administering an effective amount of the compound of the disclosure and activator intravenously. In some embodiments, the method for treating a tumor or cancer can include administering an effective amount of the compound of the disclosure intravenously, while the activator is administered orally. In some embodiments, the method for treating a tumor or cancer can include administering an effective amount of the compound of the disclosure intravenously, while the activator is administered by intratumoral injection. In some embodiments, the method can further include combinatorial administration of an additional therapeutic agent (e.g. a chemotherapeutic agent, or an immune checkpoint inhibitor).

Method for Imaging and/or for Off-Target Deactivation of Radiotherapeutics

The disclosure also pertains to a non-therapeutic method for imaging a compound according to the disclosure, in a subject, preferably a human, said non-therapeutic method comprising the steps of
(a) administering the compound according to the disclosure to the subject;
(b) imaging the compound according to the disclosure, present in the subject;
wherein the compound according to the disclosure comprises a label, typically as Construct B, wherein the label is selected from the group consisting of radionuclides, fluorescent dyes, and phosphorescent dyes. Preferably, the method of administering is as defined herein.

The disclosure also pertains to a non-therapeutic method for imaging a compound according to the disclosure, in a subject, preferably a human, said non-therapeutic method comprising the steps of
(a) prelocalizing an Activator as defined herein, in the subject;
(b) administering the compound according to the disclosure to the subject;
(c) imaging the compound according to the disclosure, present in the subject;
wherein the compound according to the disclosure comprises a label, typically as Construct B, wherein the label is selected from the group consisting of radionuclides, fluorescent dyes, and phosphorescent dyes. Preferably, step (a) is carried out by administering an Activator comprising a Targeting Agent, preferably an antibody, preferably as C^{B}, or by administering, preferably by injection, a multimeric Activator, preferably a polymer, more preferably hyaluronic acid, coupled to one or more dienes as defined herein. The prelocalization can be at any preferred site within the subject, preferably a tumour, kidney, liver, or other organs.

Preferably, the method of administering is as defined herein.

The disclosure also pertains to a non-therapeutic method for imaging an Activator as defined herein, in a subject, preferably a human, said non-therapeutic method comprising the steps of
(a) prelocalizing a compound according to the disclosure, in the subject;
(b) administering the Activator to the subject;
(c) imaging the Activator, present in the subject;
wherein the Activator comprises a label, typically as Construct B, wherein the label is selected from the group consisting of radionuclides, fluorescent dyes, and phosphorescent dyes. Preferably, step (a) is carried out by administering a compound of the disclosure comprising a Targeting Agent, preferably an antibody, preferably as C^{B}, or by administering, preferably by injection, a multimeric compound of the disclosure, preferably a polymer, more preferably hyaluronic acid, coupled to one or more compounds of the disclosure. The prelocalization can be at any preferred site within the subject, preferably a tumour, kidney, liver, or other organs. Preferably, the method of administering is as defined herein.

The disclosure also relates to a non-therapeutic method for imaging a compound according to the disclosure in a subject, preferably a human, said non-therapeutic method comprising the steps of
(a) administering a compound according to the disclosure, to the subject;
(b) administering an Activator as defined herein, to said subject; and
(c) imaging the compound according to the disclosure present in the subject;
wherein the compound according to the disclosure comprises a Targeting Agent, preferably an antibody, and a radionuclide, preferably a chelating moiety comprising a radionuclide; and wherein the Activator is preferably a Cleaving Agent as defined in WO2020/256545. Preferably, the Targeting Agent is C^{A} or part of C^{A}, and the radionuclide is C^{B} or part of C^{B}; or the Targeting Agent is C^{B} or part of C^{B}, and the radionuclide is C^{A} or part of C^{A}. Preferably, the method of administering is as defined herein.

The disclosure also relates to a non-therapeutic method for imaging an Activator as defined herein, in a subject, preferably a human, said non-therapeutic method comprising the steps of
(a) administering an Activator as defined herein, to said subject;
(b) administering a compound according to the disclosure, to the subject; and
(c) imaging the Activator present in the subject;
wherein the Activator comprises a Targeting Agent, preferably an antibody, and a radionuclide, preferably a chelating moiety comprising a radionuclide; and wherein the Activator is preferably a diene according to Formula (15) as defined herein. Preferably, the Targeting Agent is C^{A} or part of C^{A}, and the radionuclide is C^{B} or part of C^{B}; or the Targeting Agent is C^{B} or part of C^{B}, and the radionuclide is C^{A} or part of C^{A}. In this method, the compound according to the disclosure acts as a Cleaving Agent. Preferably, the method of administering is as defined herein.

The disclosure also relates to a non-therapeutic method for off-target deactivation of a radiotherapeutic in a subject, preferably a human, said non-therapeutic method comprising the steps of
(a) administering a compound according to the disclosure, to the subject; and
(b) administering an Activator as defined herein, to said subject;
wherein the compound according to the disclosure comprises a Targeting Agent, preferably an antibody, and a radionuclide, preferably a chelating moiety comprising a radionuclide; and wherein the Activator is preferably a Cleaving Agent as defined in WO2020/256545. Preferably, the Targeting Agent is C^{A} or part of C^{A}, and the radionuclide is C^{B} or part of C^{B}; or the Targeting Agent is C^{B} or part of C^{B}, and the radionuclide is C^{A} or part of C^{A}. Preferably, the method of administering is as defined herein.

The disclosure also relates to a non-therapeutic method for off-target deactivation of a radiotherapeutic in a subject, preferably a human, said non-therapeutic method comprising the steps of
(a) administering an Activator as defined herein, to said subject; and
(b) administering a compound according to the disclosure, to the subject;
wherein the Activator comprises a Targeting Agent, preferably an antibody, and a radionuclide, preferably a chelating moiety comprising a radionuclide; and wherein the Activator is preferably a diene according to Formula (15) as defined herein. Preferably, the Targeting Agent is C^{A} or part of C^{A}, and the radionuclide is C^{B} or part of C^{B}; or the Targeting Agent is C^{B} or part of C^{B}, and the radionuclide is C^{A} or part of C^{A}. In this method, the compound according to the disclosure acts as a Cleaving Agent. Preferably, the method of administering is as defined herein.

### Use of the Compounds of the Disclosure

The disclosure further pertains to the use of a compound of the disclosure in a bioorthogonal reaction, wherein preferably said use is in vitro and/or non-therapeutic. Preferably, the bioorthogonal reaction is with a diene, preferably as defined herein. Such uses may include releasing payloads in vitro, imaging in a subject, and synthesis.

### Spacers S^{P}

As the skilled person is aware, the specific structure of a spacer used in either a dienophile or diene as described herein does not typically influence whether the payload is released.

However, in some cases specific spacers are preferred. For example, if a payload is to be released, the spacer between e.g. the allylic carbon of the (E)-bicyclo[6.1.0]non-3-ene moiety and the payload is preferably a self-immolative linker. Such a linker, which is typically referred to as L^{C} herein, ensures that upon release of the end of the linker connected to said allylic carbon, a further rearrangement or reaction takes place, after which the payload is decoupled from the linker L^{C}. Below, first spacers in general are discussed, and thereafter the more specific self-immolative linkers.

In general, a spacer S′ as used herein is a moiety according to RG2, more preferably any one of the preferred and/or specific embodiments thereof.

Preferably, a spacer S′ consists of one or multiple Spacer Units S^{U} arranged linearly and/or branched and may be connected to one or more C^{B} moieties and/or one or more L^{C} or T^{R} moieties. The Spacer may be used to connect C^{B} to one T^{R} (Example A below; with reference to Formula 5a and 5b: f, e, a=1) or more T^{R} (Example B and C below; with reference to Formula 5a and 5b: f, e=1, a≥1), but it can also be used to modulate the properties, e.g. pharmacokinetic properties, of the C^{B}-T^{R}_C^{A} conjugate (Example D below; with reference to Formula 5a and 5b: one or more of c,e,g,h≥1). Thus a Spacer unit does not necessarily connect two entities together, it may also be bound to only one component, e.g. the T^{R} or L^{C}. Alternatively, the Spacer may comprise a Spacer Unit linking C^{B} to T^{R} and in addition may comprise another Spacer Unit that is only bound to the Spacer and serves to modulate the properties of the conjugate (Example F below; with reference to Formula 5a and 5b: e≥1). The Spacer may also consist of two different types of S^{U} constructs, e.g. a PEG linked to a peptide, or a PEG linked to an alkylene moiety (Example E below; with reference to Formula 5a and 5b: e≥1). For the sake of clarity, Example B depicts a S^{U} that is branched by using a multivalent branched Su. Example C depicts a S^{U} that is branched by using a linear S^{U} polymer, such as a peptide, whose side chain residues serve as conjugation groups.

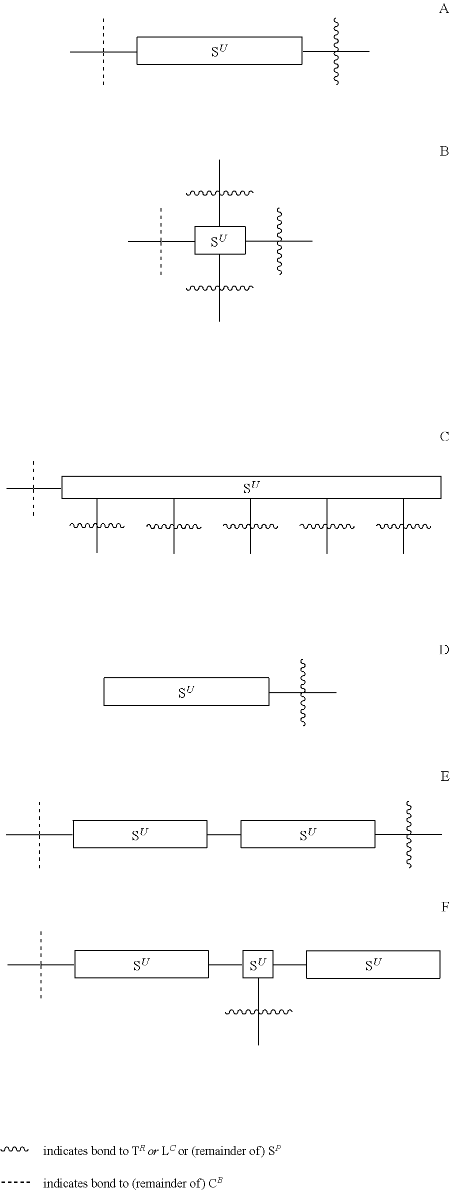

The Spacer may be bound to the Activator in similar designs such as depicted in above examples A-F.

Each individual spacer unit S^{U} may be independently selected from the group of radicals according to RG2. The Spacer Units include but are not limited to amino acids, nucleosides, nucleotides, and biopolymer fragments, such as oligo- or polypeptides, oligo- or polypeptoids, or oligo- or polylactides, or oligo- or poly-carbohydrates, varying from 2 to 200, particularly 2 to 113, preferably 2 to 50, more preferably 2 to 24 and more preferably 2 to 12 repeating units. Preferred biopolymer S^{U} are peptides. Preferably each S^{U} comprises at most 50 carbon atoms, more preferably at most 25 carbon atoms, more preferably at most 10 carbon atoms. In some embodiments the S^{U} is independently selected from the group consisting of (CH₂)r, (C₃-C₈ carbocyclo), O—(CH₂)ᵣ, arylene, (CH₂)ᵣ-arylene, arylene-(CH₂)ᵣ, (CH₂)ᵣ—(C₃-C₈ carbocyclo), (C₃-C₈ carbocyclo)-(CH₂)ᵣ, (C₃-C₅ heterocyclo), (CH₂)ᵣ—(C₃-C_{g} heterocyclo), (C₃-C₈ heterocyclo)-(CH₂)ᵣ, —(CH₂)ᵣC(O)NR′(CH₂)ᵣ, (CH₂CH₂O)ᵣ, (CH₂CH₂O)ᵣCH₂, (CH₂)ᵣC(O)NR′(CH₂ CH₂O)ᵣ, (CH₂)ᵣC(O)NR′(CH₂CH₂O)ᵣCH₂, (CH₂CH₂O)ᵣ C(O)NR′(CH₂CH₂O)ᵣ, (CH₂CH₂O)ᵣ C(O)NR′(CH₂CH₂O)ᵣCH₂, (CH₂CH₂O)ᵣC(O)NR′CH₂; wherein r is independently an integer from 1-10. As used herein, each R′ is independently selected from the group consisting of radicals according to RG1. Preferably, R′ is hydrogen. Other examples of Spacer Units S^{U} are linear or branched polyalkylene glycols such as polyethylene glycol (PEG) or polypropylene glycol (PPG) chains varying from 2 to 200, particularly 2 to 113, preferably 2 to 50, more preferably 2 to 24 and more preferably 2 to 12 repeating units. It is preferred that when polyalkylene glycols such as PEG and PPG polymers are only bound via one end of the polymer chain, that the other end is terminated with —OCH₃, —OCH₂CH₃, OCH₂CH₂CO₂H. Other polymeric Spacer Units are polymers and copolymers such as poly-(2-oxazoline), poly(N-(2-hydroxypropyl)methacrylamide) (HPMA), polylactic acid (PLA), polylactic-glycolic acid (PLGA), polyglutamic acid (PG), dextran, polyvinylpyrrolidone (PVP), poly(1-hydroxymethylethylene hydroxymethyl-formal (PHF). Other exemplary polymers are polysaccharides, glycopolysaccharides, glycolipids, polyglycoside, polyacetals, polyketals, polyamides, polyethers, polyesters. Examples of naturally occurring polysaccharides that can be used as S^{U} are cellulose, amylose, dextran, dextrin, levan, fucoidan, carraginan, inulin, pectin, amylopectin, glycogen, lixenan, agarose, hyaluronan, chondroitinsulfate, dermatansulfate, keratansulfate, alginic acid and heparin. In yet other exemplary embodiments, the polymeric S^{U} comprises a copolymer of a polyacetal/polyketal and a hydrophilic polymer selected from the group consisting of polyacrylates, polyvinyl polymers, polyesters, polyorthoesters, polyamides, oligopeptides, polypeptides and derivatives thereof. Preferred polymeric S^{U} are PEG, HPMA, PLA, PLGA, PVP, PHF, dextran, oligopeptides, and polypeptides. In some embodiments, polymers used in a S^{U} have a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, from 500 dalton to 5 kDa. Other exemplary S^{U} are dendrimers, such as poly(propylene imine) (PPI) dendrimers, PAMAM dendrimers, and glycol-based dendrimers. The S^{U} of the disclosure expressly include but are not limited to conjugates prepared with commercially available cross-linker reagents such as BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB, DTME, BMB, BMDB, BMH, BMOE, BM(PEO)₃ and BM(PEO)₄. To construct a branching Spacer one may use a S^{U} based on one or several natural or non-natural amino acids, amino alcohol, aminoaldehyde, or polyamine residues or combinations thereof that collectively provide the required functionality for branching. For example serine has three functional groups, i.e. acid, amino and hydroxyl groups and may be viewed as a combined amino acid an aminoalcohol residue for purpose of acting as a branching S^{U}. Other exemplary amino acids are lysine and tyrosine. In some embodiments, the Spacer consists of one Spacer Unit, therefore in those cases S^{P} equals Su. Preferably the Spacer consists of two, three or four Spacer Units. In some embodiments, S^{P} has a molecular weight ranging from 2 to 200 kDa, from 2 to 100 kDa, from 2 to 80 kDa, from 2 to 60 kDa, from 2 to 40 kDa, from 2 to 20 kDa, from 3 to 15 kDa, from 5 to 10 kDa, or from 500 dalton to 5 kDa. In some embodiments, the S^{P} has a mass of no more than 5000 daltons, no more than 4000 daltons, no more than 3000 daltons, no more than 2000 daltons, no more than 1000 daltons, no more than 800 daltons, no more than 500 daltons, no more than 300 daltons, no more than 200 daltons. In some aspects the S^{P} has a mass from 100 daltons, from 200 daltons, from 300 daltons to 5000 daltons. In some aspects of the S^{P} has a mass from 30, 50, or 100 daltons to 1000 daltons, from about 30, 50, or 100 daltons to 500 daltons.

Preferably, S^{P} comprises a moiety RG2a, RG2b, RG2c, or a residue of RG1f, as described herein. Preferably, said RG2a, RG2b, RG2c, or a residue of RG1f connects the S^{P} to C^{B}, L^{C}, or T^{R}.

### Self-Immolative Linkers L^{C}

L^{C} is an optional self-immolative linker, which may consist of multiple units arranged linearly and/or branched. The possible L^{C} structures, their use, position and ways of attachment of linkers L^{C}, C^{A} and the T^{R} are known to the skilled person, see for example [Papot et al., Anticancer Agents Med. Chem., 2008, 8, 618-637]. Nevertheless, preferred but non-limiting examples of self-immolative linkers L^{C} are benzyl-derivatives, such as those drawn below. There are two main self-immolation mechanisms: electron cascade elimination and cyclization-mediated elimination. The preferred example below on the left functions by means of the cascade mechanism, wherein the bond between the allylic carbon of the Trigger and the —O— or —S— attached to said carbon is cleaved, and an electron pair of Y^{C}i, for example an electron pair of NR⁶, shifts into the benzyl moiety resulting in an electron cascade and the formation of 4-hydroxybenzyl alcohol, CO₂ and the liberated payload. The preferred example in the middle functions by means of the cyclization mechanism, wherein cleavage of the bond to the NR⁶ on the side of the Trigger leads to nucleophilic attack of the amine on the carbonyl, forming a 5-ring 1,3-dimethylimidazolidin-2-one and liberating the payload. The preferred example on the right combines both mechanisms. This linker will degrade not only into CO₂ and one unit of 4-hydroxybenzyl alcohol (when Y^{C1} is O), but also into one 1,3-dimethylimidazolidin-2-one unit.

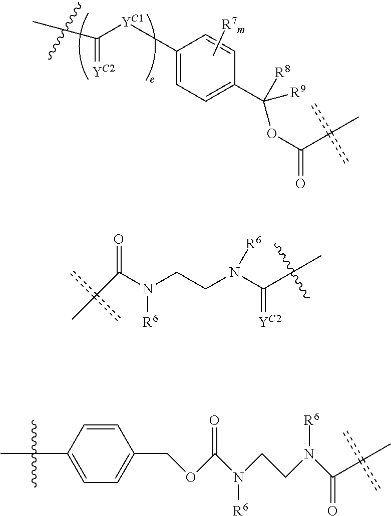

wherein the wiggly line indicates a bond to —O— or —S— on the allylic position of the trans-cyclooctene, and the double dashed line indicates a bond to C^{A}.

By substituting the benzyl groups of aforementioned self-immolative linkers L^{C}, it is possible to tune the rate of release of the payload, caused by either steric and/or electronic effects on the cyclization and/or cascade release. Synthetic procedures to prepare such substituted benzyl-derivatives are known to the skilled person (see for example [Greenwald et al, J. Med. Chem., 1999, 42, 3657-3667] and [Thornthwaite et al, Polym. Chem., 2011, 2, 773-790]. Some preferred substituted benzyl-derivatives with different release rates are drawn below.

Self-immolative linkers that undergo cyclization include but are not limited to substituted and unsubstituted aminobutyric acid amide, appropriately substituted bicyclo[2.2.1] and bicyclo[2.2.2] ring system, 2-aminophenylpropionic acid amides, and trimethyl lock-based linkers, see e.g. [Chem. Biol. 1995, 2, 223], [J. Am. Chem. Soc. 1972, 94, 5815], [J. Org. Chem. 1990, 55, 5867], the contents of which are hereby incorporated by reference. Further preferred examples of L^{C} can be found in WO2009017394(A1), U.S. Pat. No. 7,375,078, WO2015038426A1, WO2004043493, Angew. Chem. Int. Ed. 2015, 54, 7492-7509, the contents of which are hereby incorporated by reference.

Preferably the L^{C} has a mass of no more than 1000 daltons, no more than 500 daltons, no more than 400 daltons, no more than 300 daltons, or from 10, 50 or 100 to 1000 daltons, from 10, 50, 100 to 400 daltons, from 10, 50, 100 to 300 daltons, from 10, 50, 100 to 200 daltons, e.g., 10-1000 daltons, such as 50-500 daltons, such as 100 to 400 daltons.

A person skilled in the art will know that one L^{C} may be connected to another L^{C} that is bound to C^{A}, wherein upon reaction of the Activator with the Trigger T^{R}, L^{c}-L^{c}-C^{A} is released from the T^{R}, leading to self-immolative release of both L^{C} moieties and the payload. With respect to the L^{C} formulas disclosed herein, the L^{C} linking the T^{R} to the other L^{C} then does not release the payload but an L^{C} that is bound via Y^{C}i and further links to C^{A}. The skilled person will acknowledge that this principle also holds for further linkers L^{C} linked to L^{C}, e.g. L^{C}-L^{C}-L^{C}-L^{C}-C^{A}.

Preferably, if the releasable group contains a self-immolative linker, the releasable group is according to any one of Group I, Group II, Group III, and Group IV as shown below. In the structures depicted for said Groups, only bonds to Construct A and an atom (typically oxygen) on the allylic position of the trans-cyclooctene ring (part of the (E)-bicyclo[6.1.0]non-3-ene moiety) are shown for reasons of clarity, but said Construct A and said atom are part of the releasable group.

Releasable groups according to Group I are

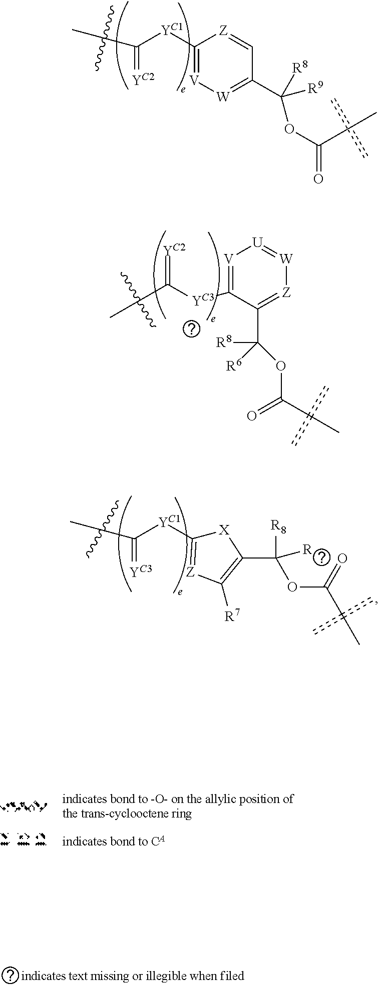

wherein the wiggly line may also indicate a bond to —S— on the allylic position of the trans-cycolooctene, wherein U, V, W, Z are each independently selected from the group consisting of —CR⁷—, and —N—, wherein e is 0 or 1, wherein X is selected from the group consisting of —O—, —S— and —NR⁶—, wherein preferably each R⁸ and R⁹ are independently selected from the group consisting of hydrogen, C₁-C₄ (hetero)alkyl, C₂-C₄ (hetero)alkenyl, and C₄₋₆ (hetero)aryl; wherein for R⁸ and R⁹ the (hetero)alkyl, (hetero)alkenyl, and (hetero)aryl are optionally substituted with a moiety selected from the group consisting of —Cl, —F, —Br, —I, —OH, —NH₂, ═O, —SH, —SO₃H, —PO₃H, —PO₄H₂ and —NO₂ and preferably contain at most two heteroatoms selected from the group consisting of —O—, —S—, —NH—, —P—, and —Si—, wherein the N, S, and P atoms are optionally oxidized. Preferably, for releasable groups of Group I both R⁸ and R⁹ are hydrogen.

The releasable group according to Group II is

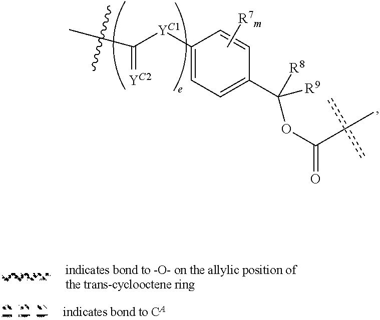

wherein the wiggly line may also indicate a bond to —S— on the allylic position of the trans-cyclooctene, wherein m is an integer between 0 and 2, preferably m is 0, wherein e is 0 or 1.

Preferably, for releasable groups of Group II both R⁸ and R⁹ are hydrogen. Preferably, for releasable groups of Group II R⁷ is methyl or isopropyl. Optionally, R⁶, R⁷, R⁸, R⁹ comprised in said Group I, and II, are —(S^{P})₁—C^{B}.

For all releasable groups according to Group I and Group II Y^{C1} is selected from the group consisting of —O—, —S—, and —NR⁶—, preferably —NR⁶—. For all linkers according to Group I, and Group II, Y^{C2} is selected from the group consisting of O and S, preferably O.

Releasable groups according to Group III are

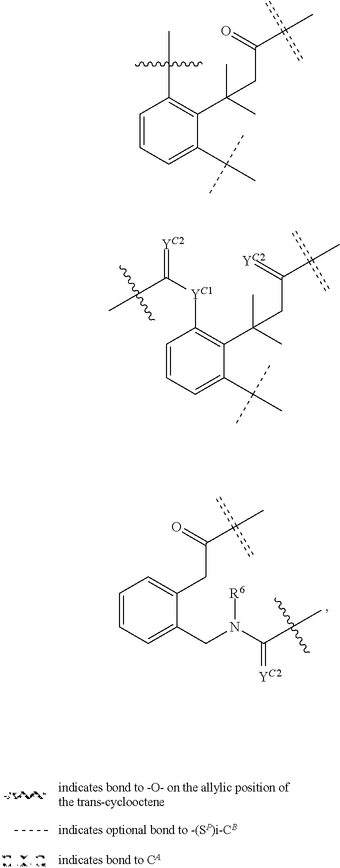

wherein the wiggly line may also indicate a bond to —S— on the allylic position of the trans-cyclooctene.

Releasable groups according to Group IV are

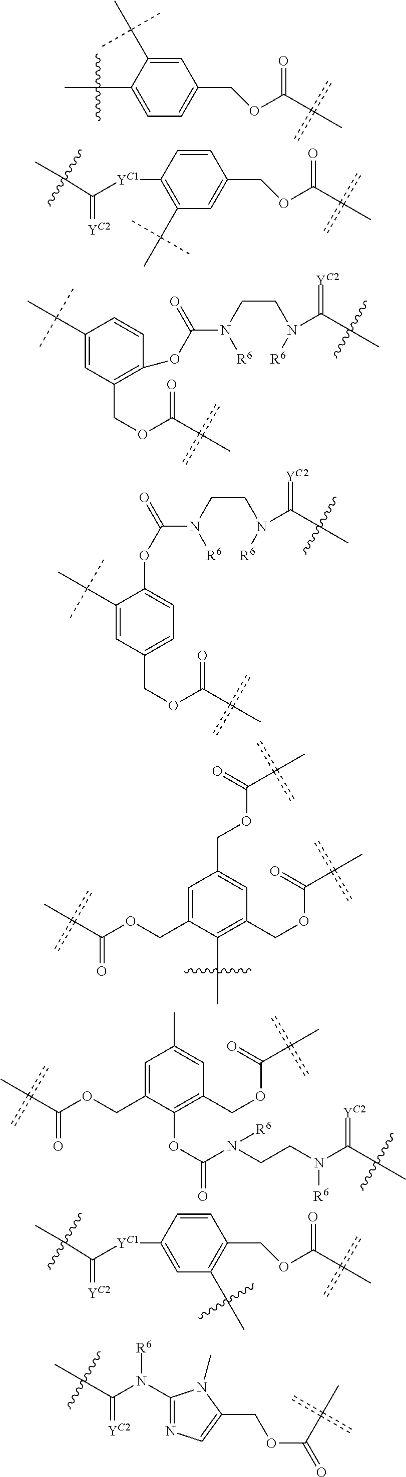

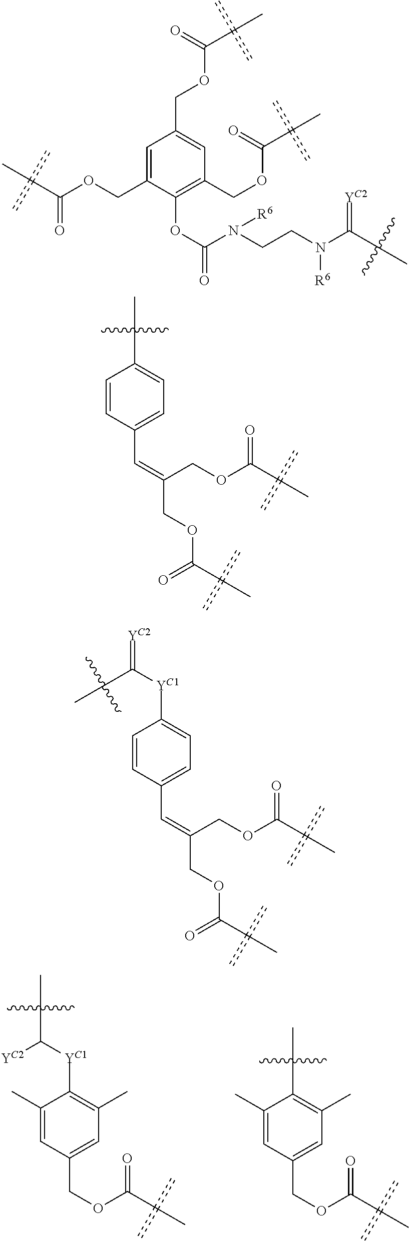

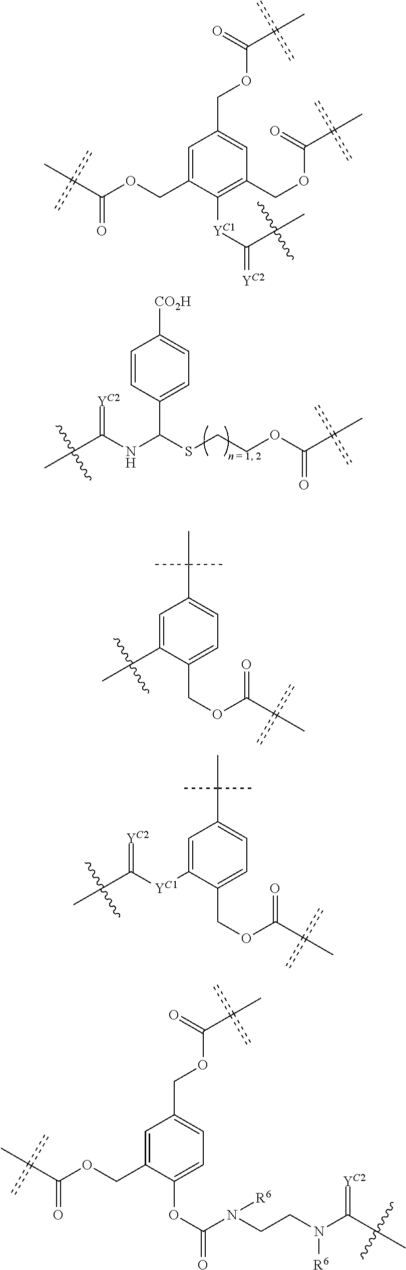

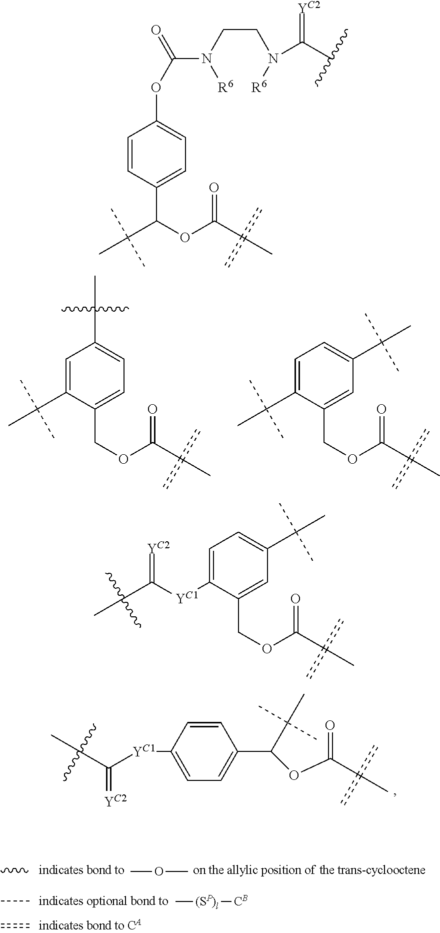

wherein the wiggly line may also indicate a bond to —S— on the allylic position of the trans-cyclooctene.

Preferably, R⁶, R⁷, R⁸, R⁹ are according to RG1 or any preferred embodiment thereof.

Preferably, R⁶, R⁷, R⁸, R⁹ used in this Section are not substituted. Most preferably, R⁶, R⁷, R⁸, R⁹ used in this Section are hydrogen.

### DEFINITIONS

The present disclosure will further be described with respect to particular embodiments and with reference to certain drawings but the disclosure is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where an indefinite or definite article is used when referring to a singular noun e.g. “a” or “an”, “the”, this includes a plural of that noun unless something else is specifically stated.

The verb “to comprise”, and its conjugations, as used in this description and in the claims is used in its non-limiting sense to mean that items following the word are included, but items not specifically mentioned are not excluded.

In addition, reference to an element by the indefinite article “a” or “an” does not exclude the possibility that more than one of the element is present, unless the context clearly requires that there is one and only one of the elements. The indefinite article “a” or “an” thus usually means “at least one”.

Thus, the scope of the expression “a device comprising means A and B” should not be limited to devices consisting only of components A and B. It means that with respect to the present disclosure, the only relevant components of the device are A and B.

The compounds disclosed in this description and in the claims may comprise one or more asymmetric centres, and different diastereomers and/or enantiomers may exist of the compounds. The description of any diene in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the disclosure of the present application is not limited to that specific enantiomer, unless stated otherwise. When the structure of a diene is depicted as a specific diastereomer, it is to be understood that the disclosure of the present application is not limited to that specific diastereomer, unless stated otherwise.

The compounds may occur in different tautomeric forms. The compounds according to the disclosure are meant to include all tautomeric forms, unless stated otherwise. When the structure of a compound is depicted as a specific tautomer, it is to be understood that the disclosure of the present application is not limited to that specific tautomer, unless stated otherwise.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the disclosure of the present application is not limited to that specific endo or exo diastereomer, unless stated otherwise.

Unless stated otherwise, the compounds of the disclosure and/or groups thereof may be protonated or deprotonated. It will be understood that it is possible that a compound may bear multiple charges which may be of opposite sign. For example, in a compound containing an amine and a carboxylic acid, the amine may be protonated while simultaneously the carboxylic acid is deprotonated.

In several formulae, groups or substituents are indicated with reference to letters such as “A”, “B”, “X”, “Y”, and various (numbered) “R” groups. In addition, the number of repeating units may be referred to with a letter, e.g. n in —(CH₂)ₙ—. The definitions of these letters are to be read with reference to each formula, i.e. in different formulae these letters, each independently, can have different meanings unless indicated otherwise.

In several chemical formulae and texts below reference is made to “alkyl”, “heteroalkyl”, “aryl”, “heteroaryl”, “alkenyl”, “alkynyl”, “alkylene”, “alkenylene”, “alkynylene”, “arylene”, “cycloalkyl”, “cycloalkenyl”, “cycloakynyl”, and the like. The number of carbon atoms that these groups have, excluding the carbon atoms comprised in any optional substituents as defined below, can be indicated by a designation preceding such terms (e.g. “C₁-C₈ alkyl” means that said alkyl may have from 1 to 8 carbon atoms). For the avoidance of doubt, a butyl group substituted with a —OCH₃ group is designated as a C₄ alkyl, because the carbon atom in the substituent is not included in the carbon count.

Unsubstituted alkyl groups have the general formula CₙH₂ₙ₊₁ and may be linear or branched. Optionally, the alkyl groups are substituted by one or more substituents further specified in this document. Examples of alkyl groups include methyl, ethyl, propyl, 2-propyl, t-butyl, 1-hexyl, 1-dodecyl, etc.

A cycloalkyl group is a cyclic alkyl group. Unsubstituted cycloalkyl groups comprise at least three carbon atoms and have the general formula CₙH₂ₙ₋₁. Optionally, the cycloalkyl groups are substituted by one or more substituents further specified in this document. Examples of cycloalkyl groups include cyclopropyl, cyclobutyl, cyclopentyl and cyclohexyl.

An alkenyl group comprises one or more carbon-carbon double bonds, and may be linear or branched. Unsubstituted alkenyl groups comprising one C═C double bond have the general formula CₙH₂ₙ₋₁. Unsubstituted alkenyl groups comprising two C═C double bonds have the general formula CₙH₂ₙ₋₃. An alkenyl group may comprise a terminal carbon-carbon double bond and/or an internal carbon-carbon double bond. A terminal alkenyl group is an alkenyl group wherein a carbon-carbon double bond is located at a terminal position of a carbon chain. An alkenyl group may also comprise two or more carbon-carbon double bonds. Examples of an alkenyl group include ethenyl, propenyl, isopropenyl, t-butenyl, 1,3-butadienyl, 1,3-pentadienyl, etc. Unless stated otherwise, an alkenyl group may optionally be substituted with one or more, independently selected, substituents as defined below.

A cycloalkenyl group is a cyclic alkenyl group. An unsubstituted cycloalkenyl group comprising one double bond has the general formula CₙH₂ₙ₋₃. Optionally, a cycloalkenyl group is substituted by one or more substituents further specified in this document. An example of a cycloalkenyl group is cyclopentenyl.

An alkynyl group comprises one or more carbon-carbon triple bonds, and may be linear or branched. Unsubstituted alkynyl groups comprising one C—C triple bond have the general formula CₙH₂ₙ₋₃. An alkynyl group may comprise a terminal carbon-carbon triple bond and/or an internal carbon-carbon triple bond. A terminal alkynyl group is an alkynyl group wherein a carbon-carbon triple bond is located at a terminal position of a carbon chain.

An alkynyl group may also comprise two or more carbon-carbon triple bonds. Unless stated otherwise, an alkynyl group may optionally be substituted with one or more, independently selected, substituents as defined below. Examples of an alkynyl group include ethynyl, propynyl, isopropynyl, t-butynyl, etc.

A cycloalkynyl group is a cyclic alkynyl group. An unsubstituted cycloalkynyl group comprising one triple bond has the general formula CₙH₂ₙ₋₅. Optionally, a cycloalkynyl group is substituted by one or more substituents further specified in this document. An example of a cycloalkynyl group is cyclooctynyl.

An aryl group refers to an aromatic hydrocarbon ring system that comprises six to twenty-four carbon atoms, more preferably six to twelve carbon atoms, and may include monocyclic and polycyclic structures. When the aryl group is a polycyclic structure, it is preferably a bicyclic structure. Optionally, the aryl group may be substituted by one or more substituents further specified in this document. Examples of aryl groups are phenyl and naphthyl. Preferably, an aryl groups is phenyl.

Arylalkyl groups and alkylaryl groups comprise at least seven carbon atoms and may include monocyclic and bicyclic structures. Optionally, the arylalkyl groups and alkylaryl may be substituted by one or more substituents further specified in this document. An arylalkyl group is for example benzyl. An alkylaryl group is for example 4-tert-butylphenyl.

Preferably, heteroaryl groups comprise five to sixteen carbon atoms and contain between one to five heteroatoms. Heteroaryl groups comprise at least two carbon atoms (i.e. at least C₂) and one or more heteroatoms N, O, P or S. A heteroaryl group may have a monocyclic or a bicyclic structure. Optionally, the heteroaryl group may be substituted by one or more substituents further specified in this document. Examples of suitable heteroaryl groups include pyridinyl, quinolinyl, pyrimidinyl, pyrazinyl, pyrazolyl, imidazolyl, thiazolyl, pyrrolyl, furanyl, triazolyl, benzofuranyl, indolyl, purinyl, benzoxazolyl, thienyl, phospholyl and oxazolyl.

Heteroarylalkyl groups and alkylheteroaryl groups comprise at least three carbon atoms (i.e. at least C₃) and may include monocyclic and bicyclic structures. Optionally, the heteroaryl groups may be substituted by one or more substituents further specified in this document.

Where an aryl group is denoted as a (hetero)aryl group, the notation is meant to include an aryl group and a heteroaryl group. Similarly, an alkyl(hetero)aryl group is meant to include an alkylaryl group and an alkylheteroaryl group, and (hetero)arylalkyl is meant to include an arylalkyl group and a heteroarylalkyl group. A C₂-C₂₄ (hetero)aryl group is thus to be interpreted as including a C₂-C₂₄ heteroaryl group and a C₆-C₂₄ aryl group. Similarly, a C₃-C₂₄ alkyl(hetero)aryl group is meant to include a C₇-C₂₄ alkylaryl group and a C₃-C₂₄ alkylheteroaryl group, and a C₃-C₂₄ (hetero)arylalkyl is meant to include a C₇-C₂₄ arylalkyl group and a C₃-C₂₄ heteroarylalkyl group.

In general, when (hetero) is placed before a group, it refers to both the variant of the group without the prefix hetero- as well as the group with the prefix hetero-. Herein, the prefix hetero- denotes that the group contains one or more heteroatoms selected from the group consisting of O, N, S, P, and Si. Preferably, the one or more heteroatoms is selected from the group consisting of O, N, S, and P. It will be understood that for any compound containing a heteroatom, the N, S, and P atoms are optionally oxidized and the N atoms are optionally quaternized. Preferably, up to two heteroatoms are consecutive, such as in for example —CH₂—NH—OCH₃ and —CH₂—O—Si(CH₃)₃. More preferably, however, the heteroatoms are not directly bound to one another.

Examples of heteroalkyls include —CH₂CH₂—O—CH₃, —CH₂CH₂—NH—CH₃, —CH₂CH₂—S(O)—CH₃, —CH═CH—O—CH₃, CH₂CH₂—NH₂, CH₂CH₂—SH, —CH₂CH₂—OH, —CH₂CH₂—COOH, —CH₂C(O)H, —C(O)HCH₃, and —Si(CH₃)₃. Preferably, a C₁-C₄ heteroalkyl contains at most 2 heteroatoms.

Herein, it will be understood that when the prefix hetero- is used for combinations of groups, the prefix hetero- only refers to the one group before it is directly placed. For example, heteroarylalkyl denotes the combination of a heteroaryl group and an alkyl group, not the combination of a heteroaryl and a heteroalkyl group.

Herein, the prefix cyclo- denotes that groups are cyclic. It will be understood that when the prefix cyclo- is used for combinations of groups, the prefix cyclo- only refers to the one group before it is directly placed. For example, cycloalkylalkenylene denotes the combination of a cycloalkylene group (see the definition of the suffix -ene below) and an alkenylene group, not the combination of a cycloalkylene and a cycloalkenylene group. In general, when (cyclo) is placed before a group, it refers to both the variant of the group without the prefix cyclo- as well as the group with the prefix cyclo-.

Herein, the suffix -ene denotes divalent groups, i.e. that the group is linked to at least two other moieties. An example of an alkylene is propylene (—CH₂—CH₂—CH₂—), which is linked to another moiety at both termini. It is understood that if a group with the suffix -ene is substituted at one position with —H, then this group is identical to a group without the suffix. For example, an alkylene attached to an —H is identical to an alkyl group. I.e. propylene, —CH₂—CH₂—CH₂—, attached to an —H at one terminus, —CH₂—CH₂—CH₂—H, is logically identical to propyl, —CH₂—CH₂—CH₃.

Herein, when combinations of groups are listed with the suffix -ene, it refers to a divalent group, i.e. that the group is linked to at least two other moieties, wherein each group of the combination contains one linkage to one of these two moieties. As such, for example alkylarylene is understood as a combination of an arylene group and an alkylene group. An example of an alkylarylene group is -phenyl-CH₂—, and an example of an arylalkylene group is —CH₂-phenyl-.

Unless indicated otherwise, a hetero group may contain a heteroatom at non-terminal positions or at one or more terminal positions. In this case, “terminal” refers to the terminal position within the group, and not necessarily to the terminal position of the entire compound.

For example, C₂ heteroalkylene may refer to —NH—CH₂—CH₂—, —CH₂—NH—CH₂—, and —CH₂—CH₂—NH—. For example, C₂ heteroalkyl may refer to —NH—CH₂—CH₃, —CH₂—NH—CH₃, and —CH₂—CH₂—NH₂.

Herein, it is understood that cyclic compounds (i.e. aryl, cycloalkyl, cycloalkenyl, etc.) are understood to be monocyclic, polycyclic or branched. It is understood that the number of carbon atoms for cyclic compounds not only refers to the number of carbon atoms in one ring, but that the carbon atoms may be comprised in multiple rings. These rings may be fused to the main ring or substituted onto the main ring. For example, C₁₀ aryl optionally containing heteroatoms may refer to inter alia a naphthyl group (fused rings) or to e.g. a bipyridyl group (substituted rings, both containing an N atom).

Unless stated otherwise, any group disclosed herein that is not cyclic is understood to be linear or branched. In particular, (hetero)alkyl groups, (hetero)alkenyl groups, (hetero)alkynyl groups, (hetero)alkylene groups, (hetero)alkenylene groups, (hetero)alkynylene groups, and the like are linear or branched, unless stated otherwise.

The general term “sugar” is herein used to indicate a monosaccharide, for example glucose (Glc), galactose (Gal), mannose (Man) and fucose (Fuc). The term “sugar derivative” is herein used to indicate a derivative of a monosaccharide sugar, i.e. a monosaccharide sugar comprising substituents and/or functional groups. Examples of a sugar derivative include amino sugars and sugar acids, e.g. glucosamine (GlcNH₂), galactosamine (GalNH₂)N-acetylglucosamine (GlcNAc), N-acetylgalactosamine (GalNAc), sialic acid (Sia) which is also referred to as N-acetylneuraminic acid (NeuNAc), and N-acetylmuramic acid (MurNAc), glucuronic acid (GlcA) and iduronic acid (ldoA). A sugar may be without further substitution, and then it is understood to be a monosaccharide. A sugar may be further substituted with at one or more of its hydroxyl groups, and then it is understood to be a disaccharide or an oligosaccharide. A disaccharide contains two monosaccharide moieties linked together. An oligosaccharide chain may be linear or branched, and may contain from 3 to 10 monosaccharide moieties.

The term “amino acid” is used herein in its normal scientific meaning. In particular, amino acids in relation to the disclosure comprise both natural and unnatural amino acids. Preferably, amino acids as used herein are selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, azidolysine, beta-alanine (bAla), 4-aminomethyl phenylalanine (Amf), 4-guanidine phenylalanine (Gnf), 4-aminomethyl-N-isopropyl phenylalanine (Iaf), 3-pyridyl alanine (Pya), 4-piperidyl alanine (Ppa), 4-aminomethyl cyclohexyl alanine (Ama), 4-aminocyclohexyl alanine (Aca), ornithine (Orn), citrulline, hydroxylysine (Hyl), allo-hydroxylysine (aHyl), 6-N-methyllysine (MeLys), desmosine (Des), isodesmosine (Ide), 2-aminoadipic acid (Aad), 3-aminoadipic acid (bAad), 2-aminobutyric acid (Abu), 4-aminobutyric acid (4Abu), 6-aminohexonic acid (Acp), 2-aminoheptanoic acid (Ahe), 2-aminoisobutyric acid (Aib), 3-aminoisobutyric acid (bAib), 2-aminopimelic acid (Apm), 2,4-diaminobutyric acid (Dbu), 2,2′-diaminopimelic acid (Dpm), 2-3-diaminopropionic acid (Dpr), N-ethylglycine (EtGly), N-ethylasparagine (EtAsn), 3-hydroxyproline (3Hyp), 4-hydroxyproline (4Hyp), allo-isoleucine (Alle), sarcosine (MeGly), N-methylisoleucine (MeIle), N-methylvaline (MeVal), norvaline (Nva), and norleucine (Nle). Preferably, an amino acid as referred to herein is a natural amino acid.

The term “protein” is herein used in its normal scientific meaning. Herein, polypeptides comprising about 10 or more amino acids are considered proteins. A protein may comprise natural, but also unnatural amino acids. The term “protein” herein is understood to comprise antibodies and antibody fragments.

The term “peptide” is herein used in its normal scientific meaning. Herein, peptides are considered to comprise a number of amino acids in a range of from 2 to 9.

The term “peptoid” is herein used in its normal scientific meaning.

An antibody is a protein generated by the immune system that is capable of recognizing and binding to a specific antigen. While antibodies or immunoglobulins derived from IgG antibodies are particularly well-suited for use in this disclosure, immunoglobulins from any of the classes or subclasses may be selected, e.g. IgG, IgA, IgM, IgD and IgE.

Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins.

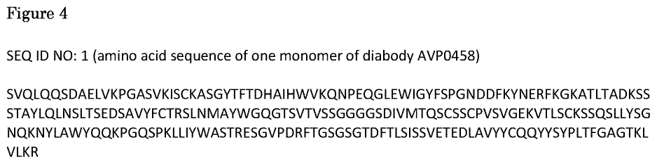

Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)₂, Fab′, Fab′-SH, F(ab′)₂, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which are hereby incorporated by reference. “Antibody fragment” refers to at least a portion of the variable region of the immunoglobulin that binds to its target, i.e. the antigen-binding region. Other embodiments use antibody mimetics as Drug D^{D} or Targeting Agent T^{T}, such as but not limited to Affimers, Anticalins, Avimers, Alphabodies, Affibodies, DARPins, and multimers and derivatives thereof, reference is made to [Trends in Biotechnology 2015, 33, 2, 65], the contents of which is hereby incorporated by reference. For the avoidance of doubt, in the context of this disclosure the term “antibody” is meant to encompass all of the antibody variations, fragments, derivatives, fusions, analogs and mimetics outlined in this paragraph, unless specified otherwise. Preferred antibodies in relation to the disclosure are CC49 and AVP0458. The amino acid sequence of one monomer of AVP0458 is depicted in FIG. 4 (SEQ ID NO: 1). AVP0458 consists of two monomers, wherein each of the two monomers has an amino acid sequence according to SEQ ID NO: 1.

A spacer is herein defined as a moiety that connects two or more elements of a compound. The terms “spacer” and “linker” are used herein interchangeably. Typically, a spacer is herein denoted as S^{P}, and the more specific self-immolative linkers as L^{C}. It will be understood that when herein, it is stated that “each individual S^{P} is linked at all ends to the remainder of the structure” this refers to the fact that the spacer S^{P} connects multiple moieties within a structure, and therefore the spacer has multiple ends by definition. The spacer S^{P} may be linked to each individual moiety via different or identical moieties that may be each individually selected. Typically, these linking moieties are to be seen to be part of spacer S^{P} itself. In case the spacer S^{P} links two moieties within a structure, “all ends” should be interpreted as “both ends”. As an example, if the spacer connects a trans-cylooctene moiety to a Construct B, then “the remainder of the molecule” refers to the trans-cylooctene moiety and Construct B, while the connecting moieties between the spacer and the trans-cyclooctene moiety and Construct B (i.e. at both ends) may be individually selected.

As used herein, an organic molecule is defined as a molecule comprising a C—H bond. Organic compound and organic molecule are used synonymously.

As used herein, an inorganic molecule is defined as any molecule not being an organic molecule, i.e. not comprising a C—H bond. It will be understood that “inorganic molecule” typically also comprises hydrogen, —COOH, etc.

As used herein, a “small molecule” is preferably a small organic molecule. In general, a small molecule has a molecular weight of at most 2 kDa, more preferably at most 1 kDa, more preferably at most 750 Da, more preferably at most 500 Da, and most preferably at most 300 Da. Preferably, a small molecule has a molecular weight of at least 15 Da, more preferably at least 50 Da, more preferably at least 75 Da, and most preferably at least 100 Da.

As used herein, “particle” is preferably defined as a microparticle or a nanoparticle.

A “primary target” as used in the present disclosure can be any molecule, which is present in an organism, tissue or cell. Preferably, a “primary target” relates to a target for a targeting agent for therapy, imaging, theranostics, diagnostics, or in vitro studies.

The term “salt thereof” means a compound formed when an acidic proton, typically a proton of an acid, is replaced by a cation, such as a metal cation or an organic cation and the like. The term “salt thereof” also means a compound formed when an amine is protonated. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted salt” means a salt that is acceptable for administration to a patient, such as a mammal (salts with counter-ions having acceptable mammalian safety for a given dosage regime). Such salts may be derived from pharmaceutically acceptable inorganic or organic bases and from pharmaceutically acceptable inorganic or organic acids.

“Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The unified atomic mass unit or Dalton is herein abbreviated to Da. The skilled person is aware that Dalton is a regular unit for molecular weight and that 1 Da is equivalent to 1 g/mol (grams per mole).

It will be understood that herein, the terms “moiety” and “group” are used interchangeably when referring to a part of a molecule.

It will be understood that when a heteroatom is denoted as —X(R′)₂—, wherein X is the heteroatom and R′ is a certain moiety, then this denotes that two moieties R′ are attached to the heteroatom.

It will be understood that when a group is denoted as, for example, —((R₅₁)₂—R₅₂)₂— or a similar notation, in which R₅₁ and R₅₂ are certain moieties, then this denotes that first, it should be written as —R₅₁—R₅₁—R₅₂—R₅₁—R₅₁—R₅₂— before the individual R₅₁ and R₅₂ moieties are selected, rather than first selecting moieties R₅₁ and R₅₂ and then writing out the formula.

It will be understood that the disclosure is divided into several Sections. It will be understood that the embodiments in each Section can be combined with embodiments from other sections, or in general with any embodiment disclosed herein. In the event that the symbols describing variables (e.g. R-groups, Formula numbers, single letters describing an integer, and the like) in a Section are identical to those of a different Section or another part of the disclosure, it will be understood that said symbols are as defined within the same Section. For example, if Section 2 and Section 3 both describe an R1-group with different definitions, R1 in Section 2 should be interpreted as defined in Section 2. Regardless of these possible identical symbols, it will be understood that the different embodiments between sections may be combined, and the symbols may be redefined (e.g. renumbered) if necessary.

As used herein, “Prodrug” refers to a compound of the disclosure wherein C^{A} is a drug, unless indicated otherwise, as the activity of the drug is significantly reduced by masking, and the drug can be reactivated if desired as described herein.

### Radical Groups (RG)

### Radical Group 1: Terminal Groups

For Radical Group 1 (RG1), the radical is selected from the group consisting of —H, —Cl, —F, —Br, —I, —OH, —NH₂, —COOH, —CONH₂, —CN, —N₃, —NCS, —SCN, —SO₃H, —PO₃H, —PO₄H₂, —NO₂, —CF₃, —CF₂H, —CFH₂, ═O, ═NH, —SH, —SO₂, —(S^{P})ᵢ—C^{B}, (hetero)alkyl, (hetero)alkenyl, (hetero)alkynyl, (hetero)cycloalkyl, (hetero)cycloalkenyl, (hetero)cycloalkynyl, (hetero)aryl, and combinations thereof. Herein, S′ is a spacer as defined herein, C^{B} is Construct B as defined herein, and i is an integer in a range of from 0 to 4, preferably i is 0 or 1.

For RG1, “combinations thereof” in particular refers to (hetero)alkylcycloalkyl, (hetero)alkylcycloalkenyl, (hetero)alkylcycloalkynyl, (hetero)cycloalkylalkyl, (hetero)cycloalkenylalkyl, (hetero)cycloalkynylalkyl, (hetero)alkenylcycloalkyl, (hetero)alkenylcycloalkenyl, (hetero)alkenylcycloalkynyl, (hetero)cycloalkylalkenyl, (hetero)cycloalkenylalkenyl, (hetero)cycloalkynylalkenyl, (hetero)alkynylcycloalkyl, (hetero)alkynylcycloalkenyl, (hetero)alkynylcycloalkynyl, (hetero)cycloalkylalkynyl, (hetero)cycloalkenylalkynyl, (hetero)cycloalkynylalkynyl, (hetero)arylalkyl, (hetero)arylalkenyl, (hetero)arylalkynyl, alkyl(hetero)aryl, alkenyl(hetero)aryl, alkynyl(hetero)aryl, cycloalkyl(hetero)aryl, cycloalkenyl(hetero)aryl, cycloalkynyl(hetero)aryl, (hetero)arylcycloalkyl, (hetero)arylcycloalkenyl, and (hetero)arylcycloalkynyl. In addition, “combinations thereof” in relation to RG1 also refers to e.g. an alkyl group substituted with one or more —Cl and/or —OH groups. As such, RG1 also comprises radicals such as —NH—CH₂—COOH (a glycine residue), which is a combination of a heteroalkyl and —COOH.

Preferably, for RG1 the radical is selected from the group RG1a consisting of —H, —Cl, —F, —Br, —I, —OH, —NH₂, —COOH, —CONH₂, —SO₃H, —PO₃H, —PO₄H₂, —NO₂, —CF₃, ═O, ═NH, —SH, —(S^{P})ᵢ—C^{B}, C₁-C₂₄ (hetero)alkyl, C₂-C₂₄ (hetero)alkenyl, C₂-C₂₄ (hetero)alkynyl, C₃-C₂₄ cycloalkyl, C₂-C₂₄ heterocycloalkyl, C₅-C₂₄ cycloalkenyl, C₃-C₂₄ heterocycloalkenyl, C₇-C₂₄ cycloalkynyl, C₅-C₂₄ (hetero)cycloalkynyl, C₆-C₂₄ aryl, C₂-C₂₄ heteroaryl, and combinations thereof.

More preferably, for RG1 the radical is selected from the group RG1b consisting of —H, —Cl, —F, —Br, —I, —OH, —NH₂, —COOH, —CONH₂, —SO₃H, —PO₃H, —PO₄H₂, —NO₂, —CF₃, ═O, ═NH, —SH, —(S^{P})ᵢ—C^{B}, C₁-C₁₂ (hetero)alkyl, C₂-C₁₂ (hetero)alkenyl, C₂-C₁₂ (hetero)alkynyl, C₃-C₁₂ cycloalkyl, C₂-C₁₂ heterocycloalkyl, C₅-C₁₂ cycloalkenyl, C₃-C₁₂ heterocycloalkenyl, C₇-C₁₂ cycloalkynyl, C₅-C₁₂ (hetero)cycloalkynyl, C₆-C₁₂ aryl, C₂-C₁₂ heteroaryl, and combinations thereof.

Even more preferably, for RG1 the radical is selected from the group RGlc consisting of —H, —Cl, —F, —Br, —I, —OH, —NH₂, —COOH, —CONH₂, —SO₃H, —PO₃H, —PO₄H₂, —NO₂, —CF₃, ═O, ═NH, —SH, —(S^{P})ᵢ—C^{B}, C₁-C₈ (hetero)alkyl, C₂-C₈ (hetero)alkenyl, C₂-C₈ (hetero)alkynyl, C₃-C₈ cycloalkyl, C₂-C₈ heterocycloalkyl, C₅-C₈ cycloalkenyl, C₃-C₅ heterocycloalkenyl, C₇-C₈ cycloalkynyl, C₅-C₈ (hetero)cycloalkynyl, C₆-C₈ aryl, C₂-C₈ heteroaryl, and combinations thereof.

More preferably still, for RG1 the radical is selected from the group RG1d consisting of —H, —Cl, —F, —Br, —I, —OH, —NH₂, —COOH, —CONH₂, —SO₃H, —PO₃H, —PO₄H₂, —NO₂, —CF₃, ═O, ═NH, —SH, —(S^{P})ᵢ—C^{B}, C₁-C₆ (hetero)alkyl, C₂-C₆ (hetero)alkenyl, C₂-C₆ (hetero)alkynyl, C₃-C₆ cycloalkyl, C₂-C₆ heterocycloalkyl, C₅-C₇ cycloalkenyl, C₃-C₅ heterocycloalkenyl, C₈ cycloalkynyl, C₆-C₇ (hetero)cycloalkynyl, phenyl, C₃-C₅ heteroaryl, and combinations thereof.

Most preferably, for RG1 the radical is selected from the group RGle consisting of —H, —Cl, —F, —Br, —I, —OH, —NH₂, —COOH, —CONH₂, —SO₃H, —PO₃H, —PO₄H₂, —NO₂, —CF₃, ═O, ═NH, —SH, —(S^{P})ᵢ—C^{B}, C₁-C₃ (hetero)alkyl, C₃-C₆ cycloalkyl, C₂-C₅ heterocycloalkyl, phenyl, C₄-C₅ heteroaryl, and combinations thereof.

In some embodiments, for RG1 the radical is a conjugation moiety, which is a chemical group that can be used for binding, conjugation or coupling of a Construct, such as Construct-B, or a Spacer, or another molecule or construct of interest. The person skilled in the art is aware of the myriad of strategies that are available for the chemoselective or -unselective or enzymatic coupling or conjugation of one molecule or construct to another.

In some embodiments, RG1 is a moiety that allows conjugation to a protein comprising natural and/or non-natural amino acids. Moieties suitable for conjugation are known to the skilled person. Conjugation strategies are for example found in [O. Boutureira, G. J. L. Bernardes, Chem. Rev., 2015, 115, 2174-2195].

If RG1 is a conjugation moiety, it is preferably selected from the group RG1f consisting of N-maleimidyl, halogenated N-alkylamido, sulfonyloxy N-alkylamido, vinyl sulfone, (activated) carboxylic acids, benzenesulfonyl halides, ester, carbonate, sulfonyl halide, thiol or derivatives thereof, C₂₋₆ alkenyl, C₂₋₆ alkynyl, C₇₋₁₈ cycloalkynyl, C₅₋₁₈ heterocycloalkynyl, bicyclo[6.1.0]non-4-yn-9-yl], C₃₋₁₂ cycloalkenyl, azido, phosphine, nitrile oxide, nitrone, nitrile imine, isonitrile, diazo, ketone, (O-alkyl)hydroxylamino, hydrazine, halogenated N-maleimidyl, aryloxymaleimides, dithiophenolmaleimides, bromo- and dibromopyridazinediones, 2,5-dibromohexanediamide, alkynone, 3-arylpropiolonitrile, 1,1-bis(sulfonylmethyl)-methylcarbonyl or elimination derivatives thereof, carbonyl halide, allenamide, 1,2-quinone, isothiocyanate, isocyanate, aldehyde, triazine, squaric acids, 2-imino-2-methoxyethyl, (oxa)norbornene, (imino)sydnones, methylsulfonyl phenyloxadiazole, aminooxy, 2-amino benzamidoxime, ethynylphosphonamidates, reactive in the Pictet-Spengler ligation and hydrazine- Pictet-Spengler (HIPS) ligation, DNA intercalators, tetrazine, and photocrosslinkers. More preferably, RG1f is N-maleimidyl.

In other embodiments RG1f is selected from the group consisting of hydroxyl, amine, halogens, vinyl pyridine, disulfide, pyridyl disulfide, sulfonyloxy, mercaptoacetamide, anhydride, sulfonylated hydroxyacetamido, sulfonyl chlorides, thiosemicarbazone, hydrazine carboxylate, and arylhydrazide. In yet other embodiments RG1f is a group that can be connected to another group by means of an enzyme, for example sortase or Tubulin tyrosine ligase.

### Radical Group 2: Connecting Groups

For Radical Group 2 (RG2), the radical is selected from the group consisting of (hetero)alkylene, (hetero)alkenylene, (hetero)alkynylene, (hetero)cycloalkylene, (hetero)cycloalkenylene, (hetero)cycloalkynylene, (hetero)arylene, RG2a, and combinations thereof.

The radicals from RG2 are optionally attached to one or more radicals according to RG1. Thus, RG2 also covers e.g. —NH—CH(CH₂OH)—C(O)— (i.e. a serine residue), which is a heteroalkylene attached to —OH and =0.

For RG2, “combinations thereof” in particular refers to alkyl(hetero)arylene, (hetero)arylalkylene, (hetero)arylalkenylene, (hetero)arylalkynylene, alkenyl(hetero)arylene, and alkynyl(hetero)arylene.

Preferably, for RG2 the radical is selected from the group consisting of C₁-C₂₄ (hetero)alkylene, C₂-C₂₄ (hetero)alkenylene, C₂-C₂₄ (hetero)alkynylene, C₃-C₂₄ cycloalkylene, C₂-C₂₄ heterocycloalkylene, C₅-C₂₄ cycloalkenylene, C₃-C₂₄ heterocycloalkenylene, C₇-C₂₄ cycloalkynylene, C₅-C₂₄ (hetero)cycloalkynylene, C₆-C₂₄ arylene, C₂-C₂₄ heteroarylene, RG2a, and combinations thereof.

More preferably, for RG2 the radical is selected from the group consisting of C₁-C₁₂ (hetero)alkylene, C₂-C₁₂ (hetero)alkenylene, C₂-C₁₂ (hetero)alkynylene, C₃-C₁₂ cycloalkylene, C₂-C₁₂ heterocycloalkylene, C₅-C₁₂ cycloalkenylene, C₃-C₁₂ heterocycloalkenylene, C₇-C₁₂ cycloalkynylene, C₅-C₁₂ (hetero)cycloalkynylene, C₆-C₁₂ arylene, C₂-C₁₂ heteroarylene, RG2a, and combinations thereof.

Even more preferably, for RG2 the radical is selected from the group consisting of C₁-C₈ (hetero)alkylene, C₂-C₈ (hetero)alkenylene, C₂-C₈ (hetero)alkynylene, C₃-C₈ cycloalkylene, C₂-C₈ heterocycloalkylene, C₅-C₈ cycloalkenylene, C₃-C₈ heterocycloalkenylene, C₇-C₈ cycloalkynylene, C₅-C₈ (hetero)cycloalkynylene, C₆-C₈ arylene, C₂-C₈ heteroarylene, RG2a, and combinations thereof.

More preferably still, for RG2 the radical is selected from the group consisting of C₁-C₆ (hetero)alkylene, C₂-C₆ (hetero)alkenylene, C₂-C₆ (hetero)alkynylene, C₃-C₆ cycloalkylene, C₂-C₆ heterocycloalkylene, C₅-C₇ cycloalkenylene, C₃-C₅ heterocycloalkenylene, C₈ cycloalkynylene, C₆-C₇ (hetero)cycloalkynylene, phenylene, C₃-C₅ heteroarylene, RG2a, and combinations thereof.

Even more preferably still, for RG2 the radical is selected from the group consisting of C₁-C₃ (hetero)alkylene, C₃-C₆ cycloalkylene, C₂-C₅ heterocycloalkylene, phenylene, C₄-C₅ heteroarylene, RG2a, and combinations thereof.

RG2a is selected from the group consisting of —O—, —S—, —SS—, —NR₄—, —N═N—, —C(O)—, —C(O)NR₄—, —OC(O)—, —C(O)O—, —OC(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(O)₂—, —OS(O)₂—, —S(O₂)O—, —OS(O)₂O—, —OS(O)₂NR₄—, —NR₄S(O)₂O—, —C(O)NR₄S(O)₂NR₄—, —OC(O)NR₄S(O)₂NR₄—, —OS(O)—, —OS(O)O—, —OS(O)NR₄—, —ONR₄C(O)—, —ONR₄C(O)O—, —ONR₄C(O)NR₄—, —NR₄OC(O)—, —NR₄OC(O)O—, —NR₄OC(O)NR₄—, —ONR₄C(S)—, —ONR₄C(S)O—, —ONR₄C(S)NR₄—, —NR₄OC(S)—, —NR₄OC(S)O—, —NR₄OC(S)NR₄—, —OC(S)—, —C(S)O—, —OC(S)O—, —OC(S)NR₄—, —NR₄C(S)—, —NR₄C(S)O—, —SS(O)₂—, —S(O)₂S—, —OS(O₂)S—, —SS(O)₂O—, —NR₄OS(O)—, —NR₄OS(O)O—, —NR₄OS(O)NR₄—, —NR₄OS(O)₂—, —NR₄OS(O)₂O—, —NR₄OS(O)₂NR₄—, —ONR₄S(O)—, —ONR₄S(O)O—, —ONR₄S(O)NR₄—, —ONR₄S(O)₂O—, —ONR₄S(O)₂NR₄—, —ONR₄S(O)₂—, —OP(O)(R₄)₂—, —S^{P}(O)(R₄)₂—, and —NR₄P(O)(R₄)₂—.

Preferably, RG2a is selected from the group consisting of —O—, —S—, —SS—, —NR₄—, —N═N—, —C(O)—, —C(O)NR₄—, —OC(O)—, —C(O)O—, —OC(O)NR₄—, —NR₄C(O)—, —NR₄C(O)O—, —NR₄C(O)NR₄—, —SC(O)—, —C(O)S—, —SC(O)O—, —OC(O)S—, —SC(O)NR₄—, —NR₄C(O)S—, —S(O)—, —S(0)₂—, —C(O)NR₄S(O)₂NR₄—, —OC(O)NR₄S(O)₂NR₄—, —OC(S)—, —C(S)O—, —OC(S)O—, —OC(S)NR₄—, —NR₄C(S)—, —NR₄C(S)O—, and —SS(O)₂—.

More preferably, for RG2 the radical is RG2b or RG2c, most preferably RG2b.

RG2b is selected from the group consisting of

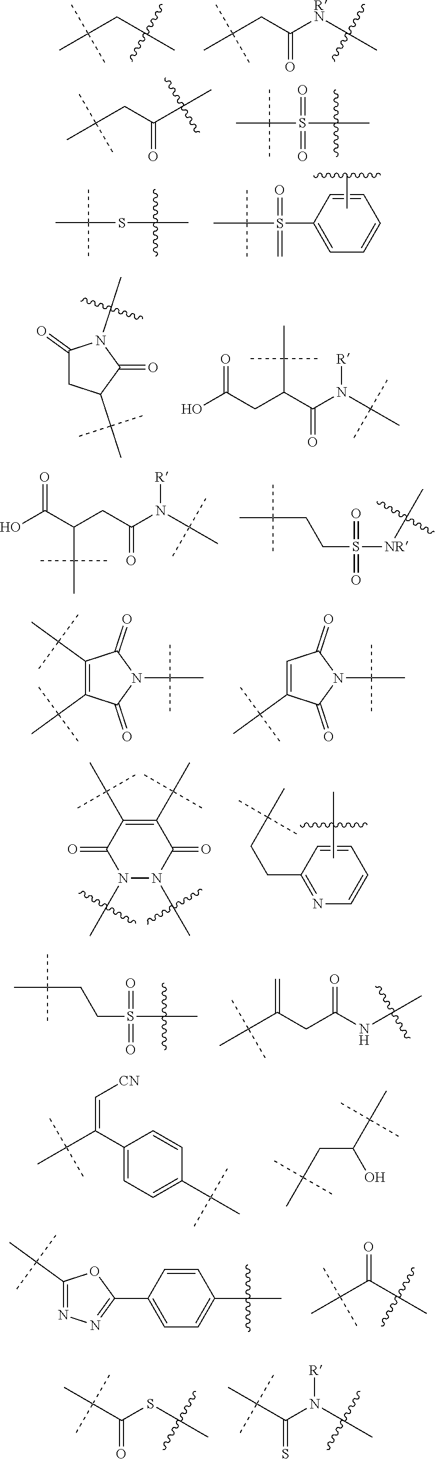

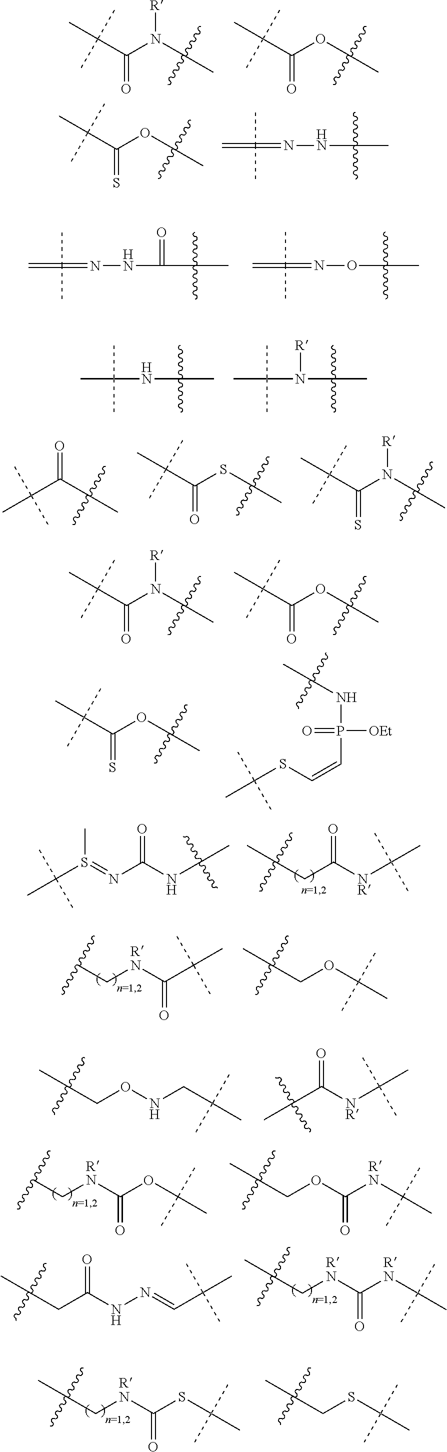

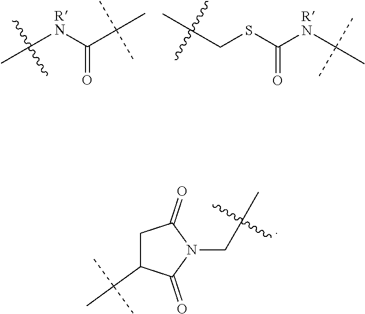

Therein, R′ is a radical according to RG1, preferably R′ is hydrogen or C₁₋₃ alkyl. The dashed and wiggly lines denote bonds to the other parts of the molecule.

RG2c is selected from the group consisting of

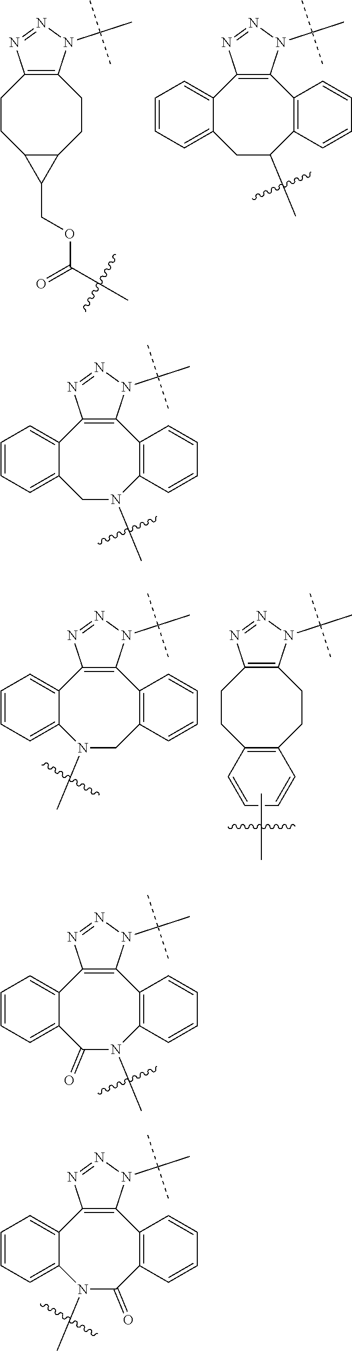

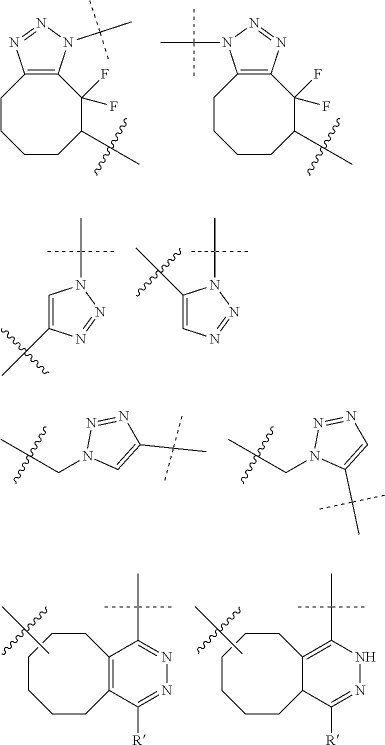

Therein, R′ is a radical according to RG1, preferably R′ is hydrogen or C₁₋₃ alkyl. The dashed and wiggly lines denote bonds to the other parts of the molecule.

### Radical Group 3: Organic Molecule

For Radical Group 3 (RG3) the radical is an organic molecule selected from the group consisting of a nucleic acid, a peptide, a protein, a carbohydrate, an aptamer, a hormone, a toxin, a steroid, a cytokine, a lipid, a small organic molecule as defined herein, a polymer, LNA, PNA, an amino acid, a peptoid, a chelating moiety, a molecule comprising a radionuclide, a fluorescent dye, a phosphorescent dye, a drug, a resin, a bead, an organic particle, a gel, an organic surface, an organometallic compound, a cell, and combinations thereof.

Preferably, for RG3 the radical is a a nucleic acid, a peptide, a protein, a carbohydrate, a lipid, a polymer, an amino acid, a chelating moiety, a drug, or a gel.

As used herein, a nucleic acid is preferably selected from the group consisting of an oligonucleotide, a polynucleotide, DNA, and RNA.

As used herein, a protein is preferably an antibody or a diabody. A preferred antibody is CC49, and a preferred diabody is AVP0458.

As used herein, a carbohydrate is preferably selected from the group consisting of a monosaccharide, an oligosaccharide, and a polysaccharide.

As used herein, a polymer is typically selected from the group consisting of polyethyleneglycol (PEG), poly(N-(2-hydroxypropyl)methacrylamide) (HPMA), polylactic acid (PLA), polylactic-glycolic acid (PLGA), polyglutamic acid (PG), polyvinylpyrrolidone (PVP), poly(1-hydroxymethylethylene hydroxymethyl-formal (PHF), copolymers of a polyacetal/polyketal and a hydrophilic polymer selected from the group consisting of polyacrylates, polyvinyl polymers, polyesters, polyorthoesters, polyamides, oligopeptides, polypeptides and derivatives thereof, oligopeptides, polypeptides, glycopolysaccharides, and polysaccharides such as dextran and hyaluronan. Preferably, a polymer as used herein is polyethylene glycol (PEG).

As used herein, a resin is preferably a polystyrene resin or an agarose resin.

As used herein, an organic particle is preferably a liposome or a polymersome.

As used herein, a chelating moiety is preferably selected from the group consisting of DTPA (diethylenetriaminepentaacetic acid), DOTA (1,4,7,10-tetraazacyclododecane-N,N′,N″,N″-tetraacetic acid), NOTA (1,4,7-triazacyclononane-N,N′,N″-triacetic acid), TETA (1,4,8,11-tetraazacyclotetradecane-N,N′,N″,N′-tetraacetic acid), OTTA (N1-(p-isothiocyanatobenzyl)-diethylenetriamine-N₁,N₂,N₃,N₃-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide), EDTA (ethylenediaminetetraacetic acid), OTAM, TACN, sarcophagine, and 3,4-HOPO-based chelators.

More preferably, herein a chelating moiety is selected from the group consisting of

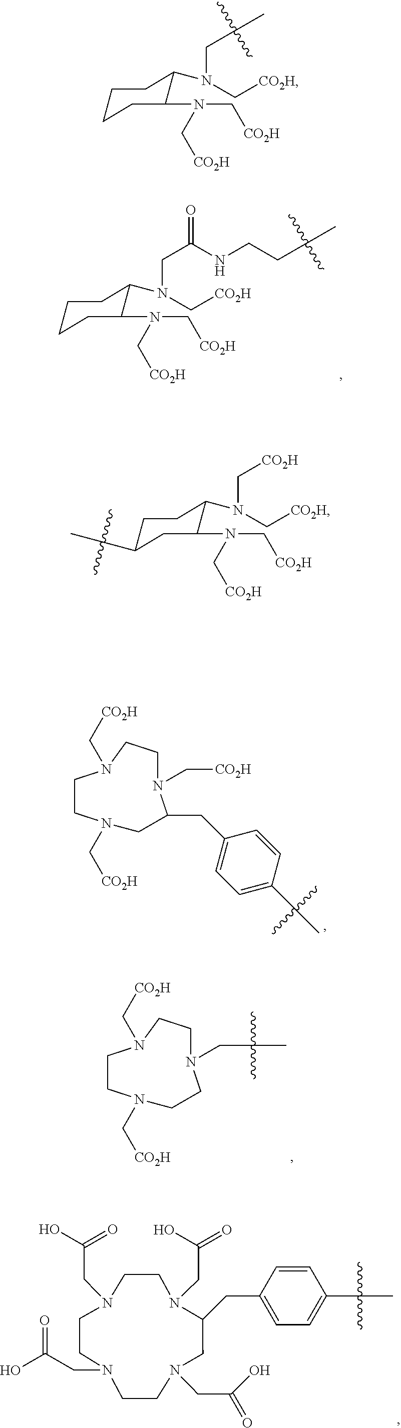 [SMILES: *CN(CC(=O)O)C1CCCCC1N(CC(=O)O)CC(=O)O ; *CCNC(=O)CN(CC(=O)O)C1CCCCC1N(CC(=O)O)CC(=O)O ; *C1CCC(N(CC(=O)O)CC(=O)O)C(N(CC(=O)O)CC(=O)O)C1 ; *c1ccc(CC2CN(CC(=O)O)CCN(CC(=O)O)CCN2CC(=O)O)cc1 ; *CN1CCN(CC(=O)O)CCN(CC(=O)O)CC1 ; *c1ccc(CC2CN(CC(=O)O)CCN(CC(=O)O)CCN(CC(=O)O)CCN2CC(=O)O)cc1]

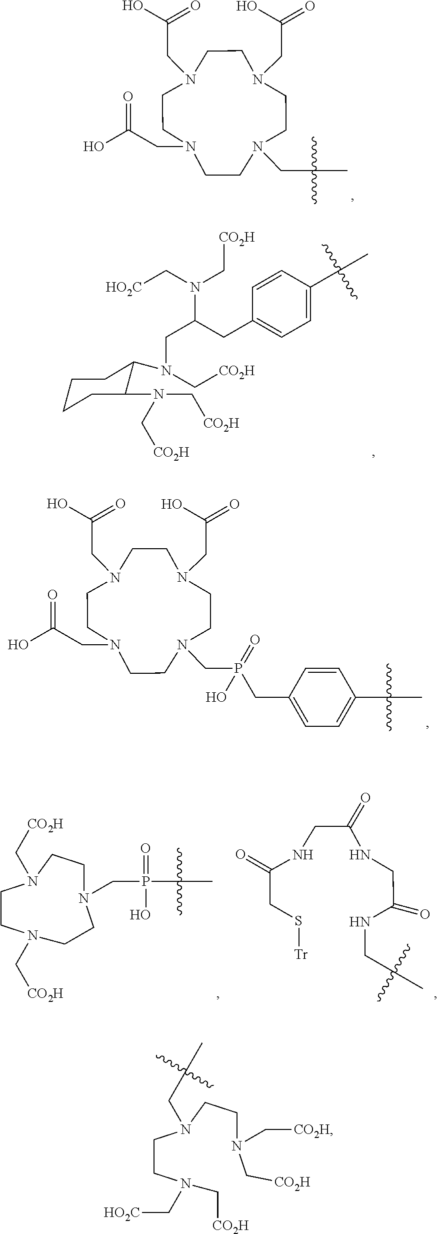

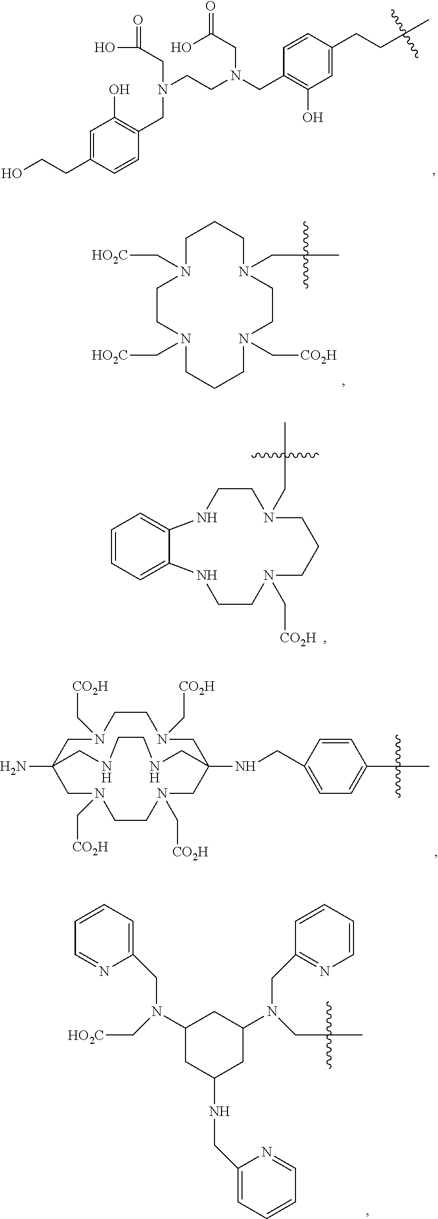 [SMILES: *CCc1ccc(CN(CCN(CC(=O)O)Cc2ccc(CCO)cc2O)CC(=O)O)c(O)c1 ; *CN1CCCN(CC(=O)O)CCN(CC(=O)O)CCCN(CC(=O)O)CC1 ; *CN1CCCN(CC(=O)O)CCNc2ccccc2NCC1 ; *c1ccc(CNC23CNCCNCC(N)(CN(CC(=O)O)CCN(CC(=O)O)C2)CN(CC(=O)O)CCN(CC(=O)O)C3)cc1 ; *CN(Cc1ccccn1)C1CC(NCc2ccccn2)CC(N(CC(=O)O)Cc2ccccn2)C1]

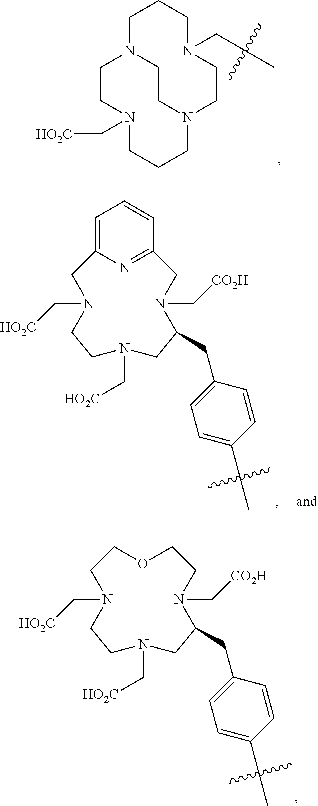 [SMILES: *CN1CCCN2CCN(CCCN(CC(=O)O)CC2)CC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1]

wherein the wiggly line denotes a bond to the remaining part of the molecule, optionally bound via —C(O)NH—, wherein the chelator moieties according to said group optionally chelate a metal, wherein the metal is preferably selected from the group consisting of ⁴⁴Sc, ⁶²Cu, ⁶⁴Cu, ⁶⁶Ga, ⁶⁷Ga, ⁶⁷Cu, ⁶⁸Ga, ⁸⁶Y, ⁸⁹Zr, ⁹⁰Y, ^{99m}Tc, ¹¹¹In, ¹⁶⁶Ho, ¹⁷⁷Lu, ¹⁹⁶Re, ¹⁸⁸Re, ²¹¹Bi, ²¹²Bi, ²¹²Pb, ²¹³Bi, ²¹⁴Bi, and ²²⁵Ac.

### Radical Group 4: Inorganic Molecule

For Radical Group 4 (RG4), the radical is an inorganic molecule selected from the group consisting of an inorganic surface, an inorganic particle, an allotrope of carbon, an inorganic drug, a radionuclide, and combinations thereof.

As used herein, an inorganic surface is preferably selected from the group consisting of chips, wafers, metal such as gold, and silica-based surfaces such as glass.

As used herein, an inorganic particle is preferably selected from the group consisting of beads, silica-based particles, polymer-based materials, and iron oxide particles. Preferably, a bead is a magnetic bead or a gold bead.

As used herein, an allotrope of carbon is preferably selected from the group consisting of fullerenes such as Buckminsterfullerene; graphite, graphene, diamond, Lonsdaleite, Q-carbon, linearn acetylenic carbon, amorphous carbon, and carbon nanotubes.

As used herein, an inorganic drug is preferably cisplatin.

### Radical Group 5: Further Terminal Groups

For RG5 the radical is:

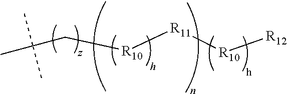

wherein the dashed line indicates a bond to the remaining part of the dienophile or diene.

For RG5, each R₁₀ is independently selected from RG2, preferably from RG2a.

For RG5, each R₁₂ is independently selected from RG2, preferably not being RG2a, RG2b, or RG2c.

For RG5, Ru is selected from RG1 or RG3, preferably RG3, more preferably a protein, polymer, or chelating moiety.

Preferably, z is an integer in a range of from 0 to 12, preferably from 0 to 10, more preferably from 0 to 8, even more preferably from 1 to 6, most preferably from 2 to 4.

Preferably, z is 0. In case the compound according to the disclosure comprises more than one moiety RG5, each z is independently selected.

Preferably, h is 0 or 1. In case the compound according to the disclosure comprises more than one moiety RG5, each h, z, and n is independently selected. Preferably, each n belonging to RG5 is an integer independently selected from a range of from 0 to 24, preferably from 1 to 12, more preferably from 1 to 6, even more preferably from 1 to 3.

Preferably, n is 1. In other preferred embodiments n is an integer in the range from 12 to 24.

Preferably, z is 0, and n is 1. In other embodiments, z is 1, and n is 1. Preferably, the moiety RG5 has a molecular weight in a range of from 100 Da to 3000 Da, preferably, in a range of from 100 Da to 2000 Da, more preferably, in a range of from 100 Da to 1500 Da, even more preferably in a range of from 150 Da to 1500 Da. Even more preferably still, the moiety RG5 has a molecular weight in a range of from 150 Da to 1000 Da, most preferably in a range of from 200 Da to 1000 Da.

Preferably, RG5 is selected from the group RG5a consisting of:

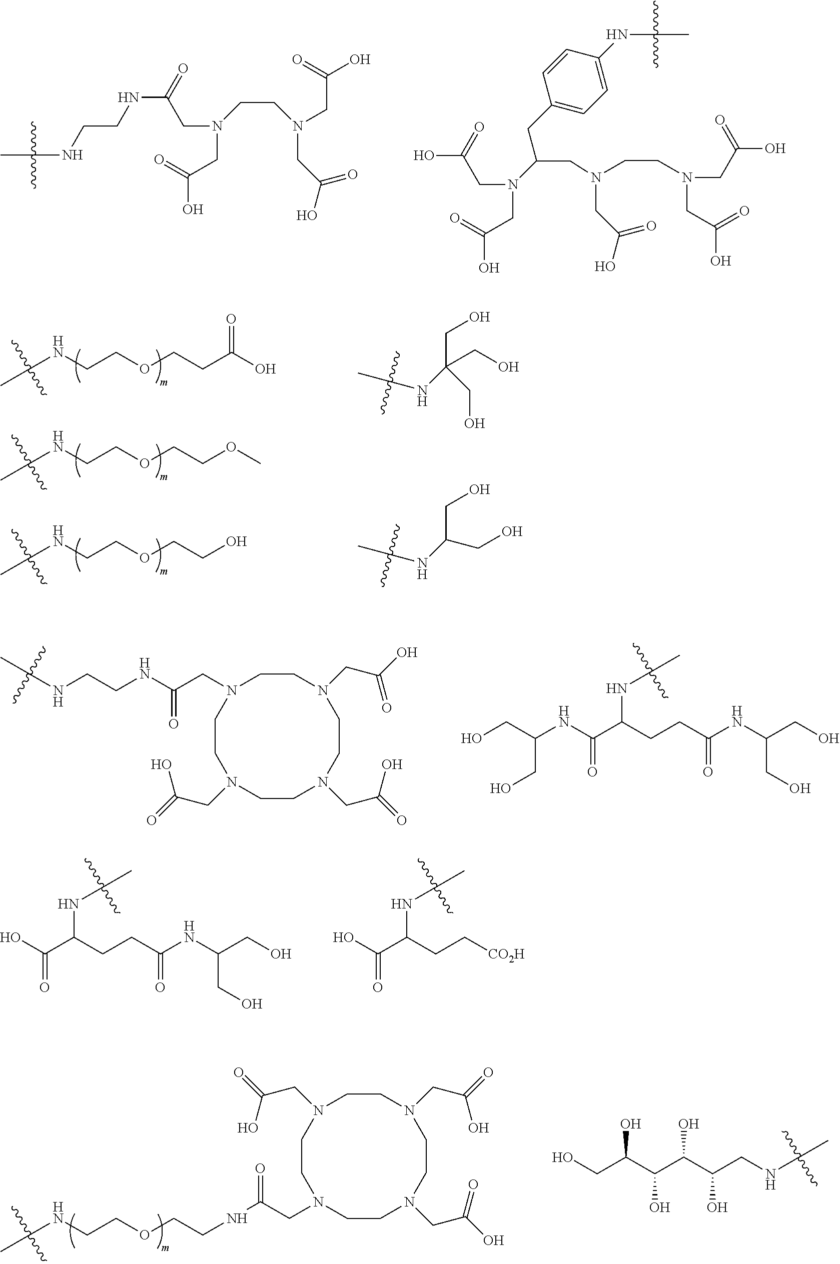

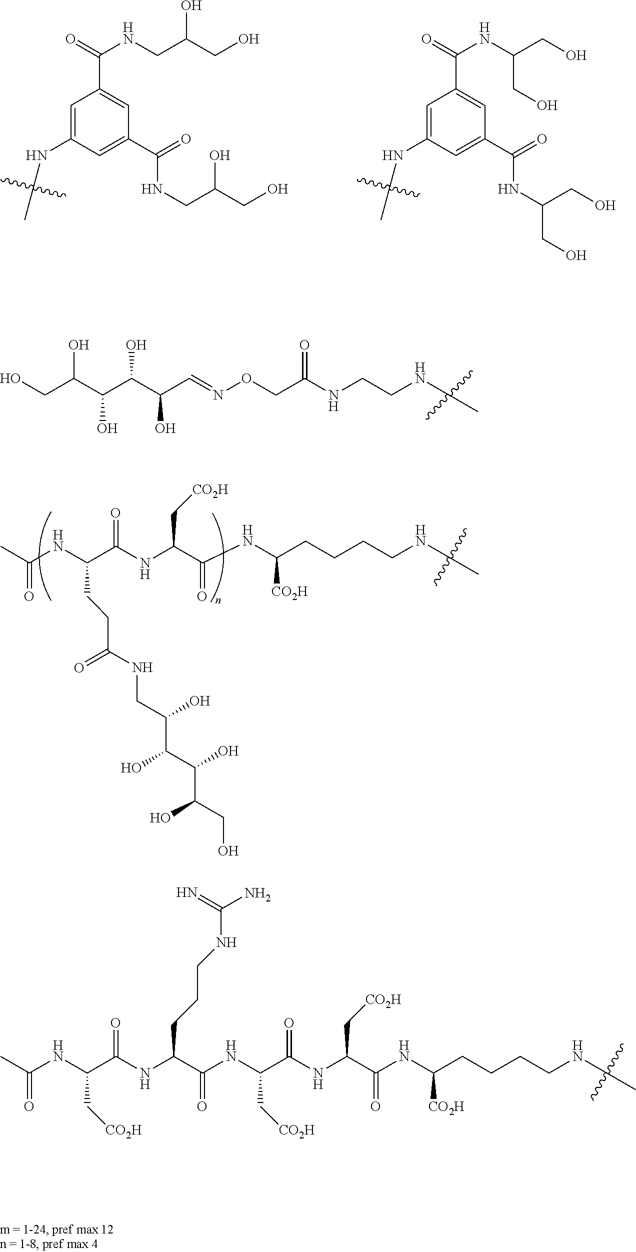

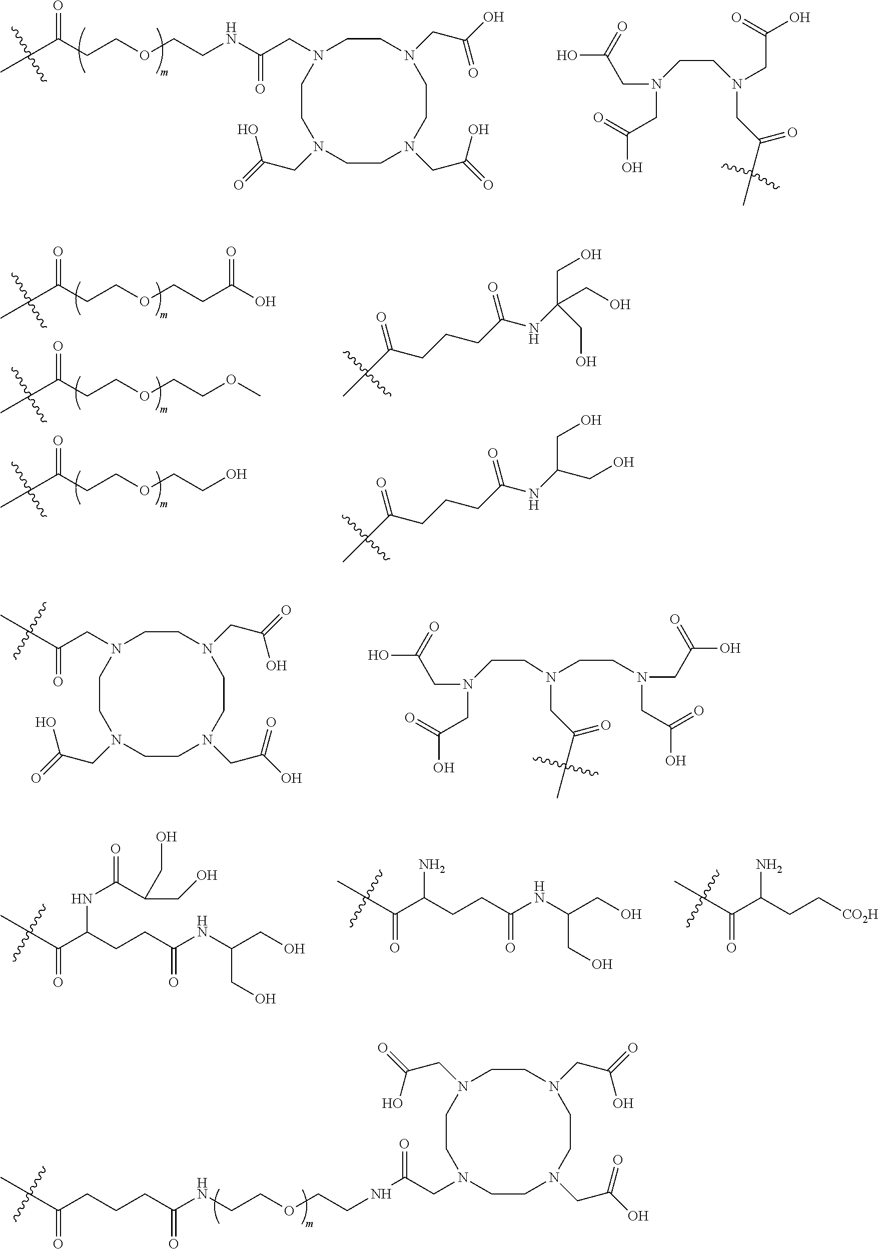

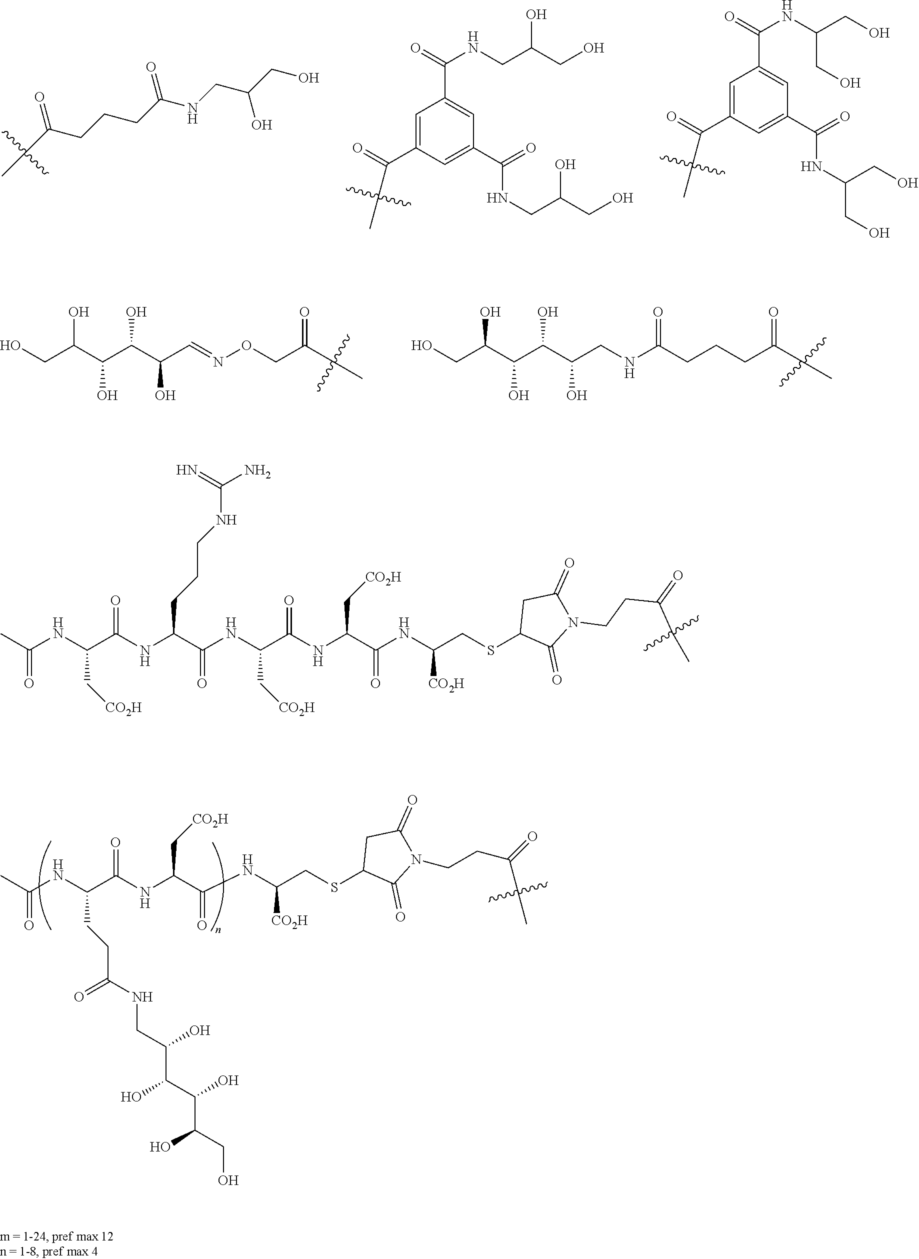

wherein the wiggly line denotes a bond to the remainder of the molecule.

It is understood that when n is more than 1, —((R₁₀)ₕ—R₁₁)ₙ—(R₁₀)ₕ—R₁₂ may be preceded by a group —(R₁₀)ₕ—R₁₁— so as to form a group —(R₁₀)ₕ—R₁₁—((R₁₀)ₕ—R₁₁)ₙ—(R₁₀)ₕ—R₁₂. It is understood that this follows from the definition of how to write out the repeating units, i.e. —((R₁₀)ₕ—R₁₁)₂— would first be written as —(R₁₀)ₕ—R₁₁—(R₁₀)ₕ—R₁₁— before R₁₀, h, and R₁₁ are independently selected.

### Targeting Agents T^{T}

A Targeting Agent, T^{T}, binds to a Primary Target. In order to allow specific targeting of the above-listed Primary Targets, the Targeting Agent T^{T} can comprise compounds including but not limited to antibodies, antibody derivatives, antibody fragments, antibody (fragment) fusions (e.g. bi-specific and tri-specific mAb fragments or derivatives), proteins, peptides, e.g. octreotide and derivatives, VIP, MSH, LHRH, chemotactic peptides, cell penetrating peptide, membrane translocation moiety, bombesin, elastin, peptide mimetics, organic compounds, inorganic compounds, carbohydrates, monosaccharides, oligosacharides, polysaccharides, oligonucleotides, aptamers, viruses, whole cells, phage, drugs, polymers, liposomes, chemotherapeutic agents, receptor agonists and antagonists, cytokines, hormones, steroids, toxins. Examples of organic compounds envisaged within the context of the present disclosure are, or are derived from, dyes, compounds targeting CAIX and PSMA, estrogens, e.g. estradiol, androgens, progestins, corticosteroids, methotrexate, folic acid, and cholesterol.

Examples of Targeting Agents of protein nature include insulin, transferrin, fibrinogen-gamma fragment, thrombospondin, claudin, apolipoprotein E, Affibody molecules such as for example ABY-025, Ankyrin repeat proteins, ankyrin-like repeat proteins, interferons, e.g. alpha, beta, and gamma interferon, interleukins, lymphokines, colony stimulating factors and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin. Examples of peptides as targeting agents include LHRH receptor targeting peptides, EC-1 peptide, RGD peptides, HER2-targeting peptides, PSMA targeting peptides, somatostatin-targeting peptides, bombesin. Other examples of targeting agents include lipocalins, such as anticalins. One particular embodiment uses Affibodies™ and multimers and derivatives.

In one embodiment antibodies are used as the T^{T}. While antibodies or immunoglobulins derived from IgG antibodies are particularly well-suited for use in this disclosure, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)₂, Fab′, Fab′-SH, F(ab′)₂, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Canc. Gen. Prot. 2013 10, 1-18], and [BioDrugs 2014, 28, 331-343], the contents of which are hereby incorporated by reference. “Antibody fragment” refers to at least a portion of the variable region of the immunoglobulin that binds to its target, i.e. the antigen-binding region. Other embodiments use antibody mimetics as T^{T}, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference. For the avoidance of doubt, in the context of this disclosure the term “antibody” is meant to encompass all of the antibody variations, fragments, derivatives, fusions, analogs and mimetics outlined in this paragraph, unless specified otherwise.

Preferably the T^{T} is selected from antibodies and antibody derivatives such as antibody fragments, fragment fusions, proteins, peptides, peptide mimetics, organic molecules, dyes, fluorescent molecules, enzyme substrates.

Preferably the T^{T} being an organic molecule has a molecular weight of less than 2000 Da, more preferably less than 1500 Da, more preferably less than 1000 Da, even more preferably less than 500 Da.

In another preferred embodiment the T^{T} is selected from antibody fragments, fragment fusions, and other antibody derivatives that do not contain a Fc domain.

In another embodiment the T^{T} is a polymer and accumulates at the Primary Target by virtue of the EPR effect. Typical polymers used in this embodiment include but are not limited to polyethyleneglycol (PEG), poly(N-(2-hydroxypropyl)methacrylamide) (HPMA), polylactic acid (PLA), polylactic-glycolic acid (PLGA), polyglutamic acid (PG), polyvinylpyrrolidone (PVP), poly(1-hydroxymethylethylene hydroxymethyl-formal (PHF). Other examples are copolymers of a polyacetal/polyketal and a hydrophilic polymer selected from the group consisting of polyacrylates, polyvinyl polymers, polyesters, polyorthoesters, polyamides, oligopeptides, polypeptides and derivatives thereof. Other examples are oligopeptides, polypeptides, glycopolysaccharides, and polysaccharides such as dextran and hyaluronan. In addition reference is made to [G. Pasut, F. M. Veronese, Prog. Polym. Sci. 2007, 32, 933-961].

In some embodiments the T^{T} can be a cell penetrating moiety, such as cell penetrating peptide. In other embodiments, the T^{T} is a polymer, particle, gel, biomolecule or another above listed T^{T} moiety and is locally injected to create a local depot of Prodrug, which can subsequently be activated by the Activator. In another embodiment the targeting agent T^{T} is a solid material such as but not limited to polymer, metal, ceramic, wherein this solid material is or is comprised in a cartridge, reservoir, depot, wherein preferably said cartridge, reservoir, depot is used for drug release in vivo. In some embodiments, the targeting agent T^{T} also acts as a Drug, denoted as D^{D}.

### Masking Moieties

A Masking Moiety as used herein may also be denoted as M^{M}. Masking moieties M^{M} can for example be an antibody, protein, peptide, polymer, polyethylene glycol, polypropylene glycol carbohydrate, aptamers, oligopeptide, oligonucleotide, oligosaccharide, carbohydrate, as well as peptides, peptoids, steroids, organic molecule, or a combination thereof that further shield the bound drug D^{D} or Prodrug. This shielding can be based on e.g. steric hindrance, but it can also be based on a non covalent interaction with the drug D^{D}. Such Masking Moiety may also be used to affect the in vivo properties (e.g. blood clearance; biodistribution, recognition by the immune system) of the drug D^{D} or Prodrug. Preferably the Masking Moiety is an albumin binding moiety. Preferably, the Masking Moiety equals a Targeting Agent. Preferably, the Masking Moiety is bound to a Targeting Agent. Preferably the Drug D^{D}, is modified with multiple M^{M}, being C^{B}, wherein at least one of the bound M^{M} is T^{T}.

Preferably the T^{R} can itself act as a Masking Moiety. For the sake of clarity, in these embodiments the size of the T^{R} without the attachment of a M^{M} is sufficient to deactivate the payload.

### Drugs

Drugs D^{D} that can be used in a Prodrug relevant to this disclosure are pharmaceutically active compounds. Preferably the pharmaceutically active compound is selected from the group consisting of cytotoxins, antiproliferative/antitumor agents, antiviral agents, antibiotics, anti-inflammatory agents, chemosensitizing agents, radiosensitizing agents, immunomodulators, immunosuppressants, immunostimulants, anti-angiogenic factors, and enzyme inhibitors.

Preferably these pharmaceutically active compounds are selected from the group consisting of antibodies, antibody derivatives, antibody fragments, proteins, aptamers, oligopeptides, oligonucleotides, oligosaccharides, carbohydrates, as well as peptides, peptoids, steroids, toxins, hormones, cytokines, and chemokines.

Most preferably, the drug is a protein, a toxin, a chelating moiety, monomethyl auristatin E, or doxorubicin; wherein preferably the chelating moiety comprises a radionuclide.

Preferably these drugs are low to medium molecular weight compounds, preferably organic compounds (e.g. about 200 to about 2500 Da, preferably about 300 to about 1750 Da, more preferably about 300 to about 1000 Da).

Exemplary cytotoxic drug types for use as conjugates to the Trigger and to be released upon IEDDA reaction with the Activator, for example for use in cancer therapy, include but are not limited to DNA damaging agents, DNA crosslinkers, DNA binders, DNA alkylators, DNA intercalators, DNA cleavers, microtubule stabilizing and destabilizing agents, topoisomerases inhibitors, radiation sensitizers, anti-metabolites, natural products and their analogs, peptides, oligonucleotides, enzyme inhibitors such as dihydrofolate reductase inhibitors and thymidylate synthase inhibitors.

Examples include but are not limited to colchinine, vinca alkaloids, anthracyclines (e.g. doxorubicin, epirubicin, idarubicin, daunorubicin), camptothecins, taxanes, taxols, vinblastine, vincristine, vindesine, calicheamycins, tubulysins, tubulysin M, cryptophycins, methotrexate, methopterin, aminopterin, dichloromethotrexate, irinotecans, enediynes, amanitins, deBouganin, dactinomycines, CC1065 and its analogs, duocarmycins, maytansines, maytansinoids, dolastatins, auristatins, pyrrolobenzodiazepines and dimers (PBDs), indolinobenzodiazepines and dimers, pyridinobenzodiazepines and dimers, mitomycins (e.g. mitomycin C, mitomycin A, caminomycin), melphalan, leurosine, leurosideine, actinomycin, tallysomycin, lexitropsins, bleomycins, podophyllotoxins, etoposide, etoposide phosphate, staurosporin, esperamicin, the pteridine family of drugs, SN-38 and its analogs, platinum-based drugs, cytotoxic nucleosides.

Other exemplary drug classes are angiogenesis inhibitors, cell cycle progression inhibitors, P13K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, and RNA polymerase inhibitors.

In some embodiments, the drug is an auristatin. Examples of auristatins include dolastatin 10, monomethyl auristatin E (MMAE), auristatin F, monomethyl auristatin F (MMAF), auristatin F hydroxypropylamide (AF HPA), auristatin F phenylene diamine (AFP), monomethyl auristatin D (MMAD), auristatin PE, auristatin EB, auristatin EFP, auristatin TP and auristatin AQ. MMAE is a preferred auristatin. Suitable auristatins are also described in U.S. Publication Nos. 2003/0083263, 2011/0020343, and 2011/0070248; PCT Application Publication Nos. WO09/117531, WO2005/081711, WO04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,879,278; 4,816,444; and 4,486,414, the disclosures of which are incorporated herein by reference in their entirety.

Exemplary drugs include the dolastatins and analogues thereof including: dolastatin A (U.S. Pat. No. 4,486,414), dolastatin B (U.S. Pat. No. 4,486,414), dolastatin 10 (U.S. Pat. Nos. 4,486,444, 5,410,024, 5,504,191, 5,521,284, 5,530,097, 5,599,902, 5,635,483, 5,663,149, 5,665,860, 5,780,588, 6,034,065, 6,323,315), dolastatin 13 (U.S. Pat. No. 4,986,988), dolastatin 14 (U.S. Pat. No. 5,138,036), dolastatin 15 (U.S. Pat. No. 4,879,278), dolastatin 16 (U.S. Pat. No. 6,239,104), dolastatin 17 (U.S. Pat. No. 6,239,104), and dolastatin 18 (U.S. Pat. No. 6,239,104), each patent incorporated herein by reference in their entirety. Exemplary maytansines, maytansinoids, such as DM-1 and DM-4, or maytansinoid analogs, including maytansinol and maytansinol analogs, are described in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 6,441,163; 6,716,821 and 7,276,497. Other examples include mertansine and ansamitocin. Pyrrolobenzodiazepines (PBDs), which expressly include dimers and analogs, include but are not limited to those described in [Denny, Exp. Opin. Ther. Patents, 10(4):459-474 (2000)], [Hartley et al., Expert Opin Investig Drugs. 2011, 20(6):733-44], Antonow et al., Chem Rev. 2011, 111(4), 2815-64]. Calicheamicins include, e.g. enediynes, esperamicin, and those described in U.S. Pat. Nos. 5,714,586 and 5,739,116. Examples of duocarmycins and analogs include CC1065, duocarmycin SA, duocarmycin A, duocarmycin B1, duocarmycin B2, duocarmycin C1, duocarmycin C2, duocarmycin D, DU-86, KW-2189, adozelesin, bizelesin, carzelesin, seco-adozelesin, CPI, CBI. Other examples include those described in, for example, U.S. Pat. Nos. 5,070,092; 5,101,092; 5,187,186; 5,475,092; 5,595,499; 5,846,545; 6,534,660; 6,548,530; 6,586,618; 6,660,742; 6,756,397; 7,049,316; 7,553,816; 8,815,226; US20150104407; 61/988,011 filed May 2, 2014 and 62/010,972 filed Jun. 11, 2014; the disclosure of each of which is incorporated herein in its entirety. Exemplary vinca alkaloids include vincristine, vinblastine, vindesine, and navelbine, and those disclosed in U.S. Publication Nos. 2002/0103136 and 2010/0305149, and in U.S. Pat. No. 7,303,749, the disclosures of which are incorporated herein by reference in their entirety. Exemplary epothilone compounds include epothilone A, B, C, D, E, and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO97/19086; WO98/08849; WO98/22461; WO98/25929; WO98/38192; WO99/01124; WO99/02514; WO99/03848; WO99/07692; WO99/27890; and WO99/28324; the disclosures of which are incorporated herein by reference in their entirety. Exemplary cryptophycin compounds are described in U.S. Pat. Nos. 6,680,311 and 6,747,021; the disclosures of which are incorporated herein by reference in their entirety. Exemplary platinum compounds include cisplatin, carboplatin, oxaliplatin, iproplatin, ormaplatin, tetraplatin. Exemplary DNA binding or alkylating drugs include CC-1065 and its analogs, anthracyclines, calicheamicins, dactinomycines, mitromycines, pyrrolobenzodiazepines, indolinobenzodiazepines, pyridinobenzodiazepines and the like. Exemplary microtubule stabilizing and destabilizing agents include taxane compounds, such as paclitaxel, docetaxel, tesetaxel, and carbazitaxel; maytansinoids, auristatins and analogs thereof, vinca alkaloid derivatives, epothilones and cryptophycins. Exemplary topoisomerase inhibitors include camptothecin and camptothecin derivatives, camptothecin analogs and non-natural camptothecins, such as, for example, CPT-11, SN-38, topotecan, 9-aminocamptothecin, rubitecan, gimatecan, karenitecin, silatecan, lurtotecan, exatecan, diflometotecan, belotecan, lurtotecan and S39625. Other camptothecin compounds that can be used in the present disclosure include those described in, for example, J. Med. Chem., 29:2358-2363 (1986); J. Med. Chem., 23:554 (1980); J. Med Chem., 30:1774 (1987). Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib, sunitinib and vatalanib. Exemplary MetAP2 inhibitors include fumagillol analogs, meaning compounds that include the fumagillin core structure. Exemplary cell cycle progression inhibitors include CDK inhibitors such as, for example, BMS-387032 and PD0332991; Rho-kinase inhibitors such as, for example, AZD7762; aurora kinase inhibitors such as, for example, AZD1152, MLN8054 and MHLN8237; PLK inhibitors such as, for example, BI 2536, BI6727, GSK461364, ON-01910; and KSP inhibitors such as, for example, SB 743921, SB 715992, MK-0731, AZD8477, AZ3146 and ARRY-520. Exemplary P13K/m-TOR/AKT signalling pathway inhibitors include phosphoinositide 3-kinase (P13K) inhibitors, GSK-3 inhibitors, ATM inhibitors, DNA-PK inhibitors and PDK-1 inhibitors. Exemplary P13 kinases are disclosed in U.S. Pat. No. 6,608,053, and include BEZ235, BGT226, BKM120, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765. Exemplary AKT inhibitors include, but are not limited to AT7867. Exemplary MAPK signaling pathway inhibitors include MEK, Ras, INK, B-Raf and p38 MAPK inhibitors. Exemplary MEK inhibitors are disclosed in U.S. Pat. No. 7,517,944 and include GDC-0973, GSK1120212, MSC1936369B, AS703026, R05126766 and R04987655, PD0325901, AZD6244, AZD8330 and GDC-0973. Exemplary B-raf inhibitors include CDC-0879, PLX-4032, and SB590885. Exemplary B p38 MAPK inhibitors include BIRB 796, LY2228820 and SB 202190.

Exemplary receptor tyrosine kinases inhibitors include but are not limited to AEE788 (NVP-AEE 788), BIBW2992 (Afatinib), Lapatinib, Erlotinib (Tarceva), Gefitinib (Iressa), AP24534 (Ponatinib), ABT-869 (linifanib), AZD2171, CHR-258 (Dovitinib), Sunitinib (Sutent), Sorafenib (Nexavar), and Vatalinib. Exemplary protein chaperon inhibitors include HSP90 inhibitors. Exemplary inhibitors include 17AAG derivatives, BIIB021, B1IIB028, SNX-5422, NVP-AUY-922 and KW-2478. Exemplary HDAC inhibitors include Belinostat (PR₄₈101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCDO103 (Mocetinostat), MS-275 (Entinostat), PCI-24781, Pyroxamide (NSC 696085), SB939, Trichostatin A and Vorinostat (SAHA). Exemplary PARP inhibitors include iniparib (BSI 201), olaparib (AZD-2281), ABT-888 (Veliparib), AG014699, CEP9722, MK 4827, KU-0059436 (AZD2281), LT-673, 3-aminobenzamide, A-966492, and AZD2461. Exemplary Wnt/Hedgehog signalling pathway inhibitors include vismodegib, cyclopamine and XAV-939. Exemplary RNA polymerase inhibitors include amatoxins. Exemplary amatoxins include alpha-amanitins, beta amanitins, gamma amanitins, eta amanitins, amanullin, amanullic acid, amanisamide, amanon, and proamanullin. Exemplary immunomodulators are APRIL, cytokines, including IL-2, IL-7, IL-10, IL12, IL-15, IL-21, TNF, interferon gamma, GMCSF, NDV-GMCSF, and agonists and antagonists of STING, agonists and antagonists of TLRs including TLR1/2, TLR3, TLR4, TLR7/8, TLR9, TLR12, agonists and antagonists of GITR, CD3, CD28, CD40, CD74, CTLA4, OX40, PD1, PDL1, RIG, MDA-5, NLRP1, NLRP3, AIM2, IDO, MEK, cGAS, and CD25, NKG2A. Other exemplary drugs include puromycins, topetecan, rhizoxin, echinomycin, combretastatin, netropsin, estramustine, cemadotin, discodermolide, eleutherobin, mitoxantrone, pyrrolobenzimidazoles (PBI), gamma-interferon, Thialanostatin (A) and analogs, CDK11, immunotoxins, comprising e.g. ricin A, diphtheria toxin, cholera toxin. In exemplary embodiments of the disclosure, the drug moiety is a mytomycin compound, a vinca alkaloid compound, taxol or an analogue, an anthracycline compound, a calicheamicin compound, a maytansinoid compound, an auristatin compound, a duocarmycin compound, SN38 or an analogue, a pyrrolobenzodiazepine compound, a indolinobenzodiazepine compound, a pyridinobenzodiazepine compound, a tubulysin compound, a non-natural camptothecin compound, a DNA binding drug, a kinase inhibitor, a MEK inhibitor, a KSP inhibitor, a P13 kinase inhibitor, a topoisomerase inhibitor, or analogues thereof. In one preferred embodiment the drug is a non-natural camptothecin compound, vinca alkaloid, kinase inhibitor, (e.g. P13 kinase inhibitor: GDC-0941 and PI-103), MEK inhibitor, KSP inhibitor, RNA polymerase inhibitor, PARP inhibitor, docetaxel, paclitaxel, doxorubicin, dolastatin, calicheamicins, SN38, pyrrolobenzodiazepines, pyridinobenzodiazepines, indolinobenzodiazepines, DNA binding drugs, maytansinoids DM1 and DM4, auristatin MMAE, CC1065 and its analogs, camptothecin and its analogs, SN-38 and its analogs.

In another preferred embodiment the drug is selected from DNA binding drugs and microtubule agents, including pyrrolobenzodiazepines, indolinobenzodiazepines, pyridinobenzodiazepines, maytansinoids, maytansines, auristatins, tubulysins, duocarmycins, anthracyclines, taxanes.

In another preferred embodiment the drug is selected from colchinine, vinca alkaloids, tubulysins, irinotecans, an inhibitory peptide, amanitin and deBouganin.

In another preferred embodiment the drug is a radioactive moiety, said moiety comprising a radioactive isotope for radiation therapy. A radionuclide used for therapy is preferably an isotope selected from the group consisting of ²⁴Na, ³²P, ³³P, ⁴⁷Sc, ⁵⁹Fe, ⁶⁷Cu, ⁷⁶As, ⁷⁷As, ⁸⁰Br, ⁸²Br, ⁸⁹Sr, ⁹⁰Nb, ⁹⁰Y, ¹⁰³Ru, ¹⁰⁵Rh, ¹⁰⁹Pd, ¹¹¹Ag, ¹¹¹I, ¹²¹Sn, ¹²⁷Te, ¹³¹I, ¹⁴⁰La, ¹⁴¹Ce, ¹⁴²Pr, ¹⁴³Pr, ¹⁴⁴Pr, ¹⁴⁹Pm, ¹⁴⁹Tb, ¹⁵¹Pm, ¹⁵³Sm, ¹⁵⁹Gd, ¹⁶¹Tb, ¹⁶⁵Dy, ¹⁶⁶Dy, ¹⁶⁶Ho, ¹⁶⁹Er, ¹⁷²Tm, ¹⁷⁵Y_{b}, ¹⁷⁷Lu, ¹⁸⁶Re, ¹⁸⁸Re, ¹⁹⁸Au, ¹⁹⁹Au, ²¹¹At, ²¹¹Bi, ²¹²Bi, ²¹²Pb, ²¹³Bi, ²¹⁴Bi, ²²³Ra ²²⁴Ra, ²²⁵Ac, and ²²⁷Th. When the radioactive moiety is intended to comprise a metal, such as ¹⁷⁷Lu, such radiometal is preferably provided in the form of a chelate. In such a case the radioactive moiety preferably comprises a structural moiety capable of forming a coordination complex with such a metal. A good example hereof are macrocylic lanthanide(III) chelates derived from 1,4,7,10-tetraazacyclododecane-1,4,7,10-tetraacetic acid (H₄dota). Preferably, the structural moiety capable of forming a coordination complex with such a metal is a chelating moiety as defined herein. In other embodiments the radioactive moiety comprises a prosthetic group (i.e. a phenol) that is bound by a non-metal radionuclide, such as 13′I.

Drugs optionally include a (portion of a) membrane translocation moiety (e.g. adamantine, poly-lysine/arginine, TAT, human lactoferrin) and/or a targeting agent (against e.g. a tumor cell receptor) optionally linked through a stable or labile linker. Exemplary references include: Trends in Biochemical Sciences, 2015, 40, 12, 749; J. Am. Chem. Soc. 2015, 137, 12153-12160; Pharmaceutical Research, 2007, 24, 11, 1977.

It will further be understood that, in addition to one or more targeting agents (or C^{B}) that may be attached to the Trigger or Linker L^{C} a targeting agent T^{T} may optionally be attached to a drug, optionally via a spacer S^{P}.

Alternatively, it will be further understood that the targeting agent (or C^{B}) may comprise one or more additional drugs which are bound to the targeting agent by other types of linkers, e.g. cleavable by proteases, pH, thiols, or by catabolism.

It will be understood that chemical modifications may also be made to the desired compound in order to make reactions of that compound more convenient for purposes of preparing conjugates of the disclosure.

Drugs containing an amine functional group for coupling to the Trigger include mitomycin-C, mitomycin-A, daunorubicin, doxorubicin, aminopterin, actinomycin, bleomycin, 9-amino camptothecin, N8-acetyl spermidine, 1-(2 chloroethyl)1,2-dimethanesulfonyl hydrazide, tallysomycin, cytarabine, dolastatins (including auristatins) and derivatives thereof. Drugs containing a hydroxyl function group for coupling to the Trigger include etoposide, camptothecin, taxol, esperamicin, 1,8-dihydroxy-bicyclo[7.3.1]trideca-4-9-diene-2,6-diyne-13-one (U.S. Pat. No. 5,198,560), podophyllotoxin, anguidine, vincristine, vinblastine, morpholine-doxorubicin, n-(5,5-diacetoxy-pentyl)doxorubicin, and derivatives thereof. Drugs containing a sulfhydryl functional group for coupling to the Trigger include esperamicin and 6-mecaptopurine, and derivatives thereof.

### EXAMPLES

### Example 1: Synthesis of s-TCO Derivatives

### General Procedure A: Photoisomerization

FEP tubing (24 mL of 1/16th inch) coiled around a quartz water cooled jacket containing a 36W UV-C lamp is equilibrated with 50% Et₂O in heptane. A 12 g ISCO cartridge is charged with 5 g of silica (on the bottom), 5 g of 9.5% AgNO₃ on silica (in the middle) and 2 g silica (on top) and equilibrated with 50% Et₂O in heptane. The cis-cyclooctene (1 eq) and methyl benzoate (2 eq) are dissolved in 20 mL 1:1 Et₂O:heptane. The substrate is cycled through the reactor and column at 5 mL/min (4.8 min residence time) and column while bubbling with N₂ for 15 minutes prior to starting the reaction by turning the lamp on. After 3 hours, the column is flushed with Et₂O. The silica is subsequently treated with Et₂O with 12 eq NH₃ (aq) and filtered. The silica is extracted once more with CH₂Cl₂:NH₃ and the combined extracts are concentrated and purified by silica column chromatography (EtOAc in heptanes) and/or purified by preparative HPLC to yield both the axial isomer and the equatorial isomer of the s-TCO in pure form after lyophilization.

### Examples 1.1-1.7—Synthesis of 1,5-s-TCO Derivatives

### Example 1.1—Ethyl (1R,8S,Z)-bicyclo[6.1.0]non-4-ene-9-carboxylate (1.1)

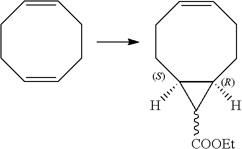

To a mixture of 1,5-cyclooctadiene (20 mL, 163.06 mmol) and Rh₂(OAc)₄ (35 mg, 0.08 mmol) in dichloromethane (DCM, 150 mL) at 0 °C, a solution of ethyl diazoacetate (8.0 mL, 76.07 mmol) in DCM (60 mL) was added dropwise for 2 hours. The reaction mixture was stirred at 20 °C for 18 hours. After which DCM was evaporated in vacuo, and the residue was purified by silica gel column chromatography (eluent: from 100% heptane to 10% ethyl acetate (EtOAc) in heptane) to give 1.1 as a colorless oil as a mixture of endo/exo isomers (9.06 g, 60% yield). ¹H NMR (400 MHz, CDCl₃) δ 5.74-5.45 (m, 2H), 4.26-3.93 (m, 2H), 2.62-2.45 (m, 1H), 2.40-2.34 (m, 2H), 2.25-2.14 (m, 2H), 2.14-1.99 (m, 2H), 1.93-1.78 (m, 2H), 1.46-1.34 (m, 2H), 1.29-1.24 (m, 3H) ppm. ESI-MS: m/z Calc. for C₁₂H₁₈O₂ 194.13 Da; Obs. [M+H]⁺ 195.04 Da.

### Example 1.2—(1R,8S,Z)-Bicyclo[6.1.0]non-4-ene-9-carboxylic acid (1.2)

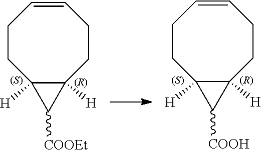

To a solution of compound 1.1 (9.0 g, 46.32 mmol) in methanol (200 mL) was added dropwise a solution of NaOH (36 g, 900 mmol) in water (300 mL). The reaction mixture was stirred at room temperature for 48 hours under N₂ atmosphere. After which methanol was removed in vacuo, and pH of the residue was adjusted to 2 with HCl solution (2 M). The residue was extracted with EtOAc (3×150 mL) and the combined organic layers were dried over Na₂SO₄ anh., filtered and concentrated. The crude product was used directly without further purification as a mixture of endo/exo isomers. ¹H NMR (400 MHz, CDCl₃) δ 5.77-5.54 (m, 2H), 2.62-2.43 (m, 1H), 2.40-2.28 (m, 2H), 2.28-2.18 (m, 2H), 2.17-2.01 (m, 2H), 1.98-1.80 (m, 1H), 1.70-1.64 (m, 1H), 1.58-1.46 (m, 2H) ppm. ESI-MS: m/z Calc. for C₁₀H₁₄O₂ 166.10 Da; Obs. [M+H]⁺ 167.04 Da.

### Example 1.3—(1S,2R,5R,6R,9S)-5-Iodo-7-oxatricyclo[4.3.2.0^{2,9}]undecan-8-one (1.3)

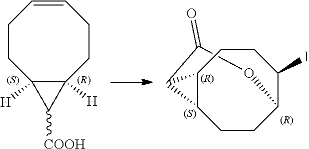

To a solution of compound 1.2 (7.48 g, 45 mmol) in DCM (55 mL) was added water (25 mL) and NaHCO₃ (7.50 g, 89.28 mmol). After stirring at room temperature for 30 minutes, a mixture of KI (7.5 g, 45.18 mmol) and 12 (11.10 g, 43.90 mmol) was added in small portions within 1 hour to the reaction mixture, and the dark brown mixture was stirred at room temperature for 18 hours under N₂ atmosphere. Na₂SO₃ sat. aq. was added to quench the excess I₂, and the reaction mixture was extracted with EtOAc (3×100 mL). The combined organic layers were dried over Na₂SO₄, filtered and concentrated. The residue was purified by silica gel column chromatography (Eluent: from 20% EtOAc in petroleum ether (PE) to 50% EtOAc in PE) to afford compound 1.3 in 30% yield (4.0 g, 13.70 mmol). ¹H NMR (400 MHz, CDCl₃) δ 4.72-4.66 (m, 1H), 4.41-4.31 (m, 1H), 2.75-2.59 (m, 1H), 2.59-2.42 (m, 2H), 2.34-2.14 (m, 3H), 2.11-1.86 (m, 3H), 1.70-1.61 (m, 1H), 1.62-1.44 (m, 1H) ppm. 13C NMR (101 MHz, CDCl₃) δ 179.08, 73.05, 29.89, 27.11, 26.16, 24.64, 22.79, 21.53, 19.83, 18.70 ppm. ESI-MS: m/z Calc. for C₁₀H₁₃IO₂ 292.00 Da; Obs. [M+H]⁺ 293.04 Da.

### Example 1.4—(1S,2R,6R,9S,Z)-7-Oxatricyclo[4.3.2.0^{2,9}]undec-4-en-8-one (1.4)

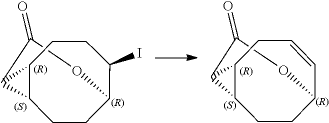

The iodolactone compound 1.3 (0.14 g, 0.48 mmol) was dissolved in toluene (6 mL), and DBU (0.15 mL, 1.00 mmol) was added. The mixture was heated to 55 °C and allowed to stand for 18 hours under N₂ atmosphere. After cooling, the reaction mixture was diluted with brine (15 mL) and extracted with EtOAc (3×15 mL). The combined organic layers were dried over Na₂SO₄, filtered and concentrated. The residue was purified with silica gel column chromatography (Eluent: from 10% EtOAc in PE to 25% EtOAc in PE) to give compound 1.4 (60 mg, 0.36 mmol) in yield of 75%. ¹H NMR (400 MHz, CDCl₃) δ 5.88 (ddd, J=12.3, 7.8, 4.8 Hz, 1H), 5.39 (dddd, J=12.0, 6.1, 2.4, 1.2 Hz, 1H), 4.87 (td, J=5.8, 1.7 Hz, 1H), 2.93-2.64 (m, 2H), 2.47-2.28 (m, 1H), 2.22-2.10 (m, 1H), 2.04 (t, J=1.1 Hz, 1H), 2.00-1.91 (m, 1H), 1.89-1.78 (m, 1H), 1.72-1.57 (m, 1H), 1.54-1.43 (m, 1H) ppm. ¹³C NMR (126 MHz, CDCl₃) δ 174.58, 128.21, 126.61, 75.93, 30.32, 25.55, 23.89, 23.69, 21.04, 19.38 ppm. ESI-MS: m/z Calc. for C₁₀H₁₂O₂ 164.08 Da; Obs. [M+H]⁺ 165.04 Da.

### Example 1.5—Methyl (1R,5R,8S,9S,Z)-5-hydroxybicyclo[6.1.0]non-3-ene-9-carboxylate (1.5)

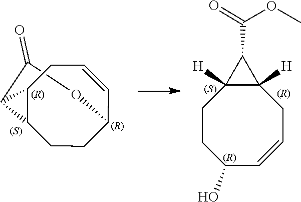

The bicyclic olefin compound 1.4 (50 mg, 0.30 mmol) was dissolved in toluene (8 mL) and methanol (3 mL), and KOH (50 mg, 0.90 mmol) was added. The mixture was heated to 55 °C and allowed to stand for 2 hours under N₂ atmosphere. After cooling the solvent was removed in vacuo, and the residue was dissolved in dimethylformamdie (DMF, 3 mL), and iodomethane (0.15 g, 1.05 mmol) was added to the reaction mixture. The mixture was stirred at 35 °C for 4 hours before diluting with EtOAc (15 mL), and washed with brine (3×10 mL). The organic layer was dried over Na₂SO₄, filtered and concentrated. The residue was purified by silica gel column chromatography (Eluent: from 10% EtOAc in PE to 25% EtOAc in PE) to give compound 1.5 (40 mg, 0.20 mmol) in a yield of 67%. ¹H NMR (400 MHz, CDCl₃) δ 5.65-5.47 (m, 1H), 5.46-5.35 (m, 1H), 5.09-4.98 (m, 1H), 3.66 (s, 3H), 2.74-2.54 (m, 1H), 2.53-2.37 (m, 1H), 2.05-1.87 (m, 3H), 1.87-1.74 (m, 1H), 1.74-1.64 (m, 1H), 1.63-1.49 (m, 1H), 1.46-1.31 (m, 1H), 1.33-1.19 (m, 1H) ppm. ¹³C NMR (126 MHz, CDCl₃) δ 172.12, 132.52, 127.46, 68.62, 51.35, 36.99, 24.94, 24.61, 23.04, 21.55, 18.45 ppm. ESI-MS: m/z Calc. for C₁₁H₁₆O₃ 196.11 Da; Obs. [M+H]⁺ 197.12 Da.

### Example 1.6—Methyl (1R,5R,8S,9S,E)-5-hydroxybicyclo[6.1.0]non-3-ene-9-carboxylate (1.6a, 1.6b; 2 isomers with the allylic release-OH either in axial or equatorial position)

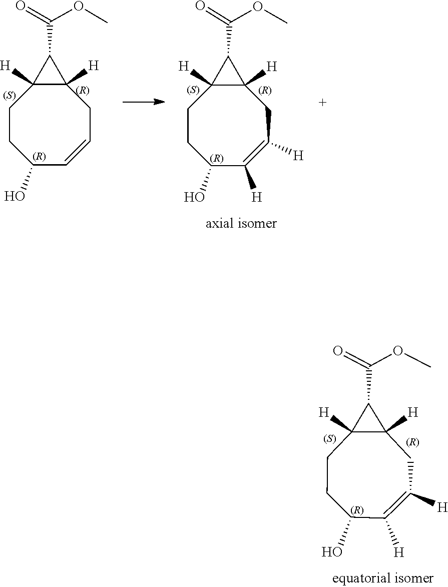

Compound 1.5 (190 mg, 0.97 mmol) was subjected to the General Procedure A to give axial isomer compound 1.6a (8 mg, 0.04 mmol, in 4% yield) and equatorial isomer compound 1.6b (10 mg, 0.05 mmol, in 5% yield).

Axial Isomer (1.6a) ¹H NMR (400 MHz, CDCl₃) δ 6.49 (ddd, J=17.1, 7.4, 7.2 Hz, 1H), 5.65 (dt, J=17.1, 3.5 Hz, 1H), 4.66 (s, 1H), 3.69 (s, 3H), 2.74 (m, 1H), 2.37 (m, 1H), 2.10 (dtd, J=9.3, 9.1, 1.8 Hz, 1H), 2.00 (m, 1H), 1.78 (t, J=9.1 Hz, 1H), 1.7-1.5 (m, 3H), 1.07 (dtd, J=10.8, 9.1, 5.0 Hz, 1H) ppm. ¹³C NMR (101 MHz, CDCl₃) δ 172.12, 138.13, 132.76, 70.72, 51.22, 37.34, 35.73, 26.11, 24.55, 23.99, 20.03 ppm.

### Equatorial Isomer (1.6b)

¹H NMR (400 MHz, CDCl₃) δ 5.76 (dd, J=17.3, 7.2 Hz, 1H), 5.40 (ddd, J=17.3, 11.5, 3.3 Hz, 1H), 4.74 (t, J=7.2 Hz, 1H), 3.69 (s, 3H), 2.67 (m, 1H), 2.44 (m, 1H), 2.2-1.5 (m, 6H), 1.17 (m, 1H) ppm. ¹³C NMR (101 MHz, CDCl₃) δ 172.16, 138.31, 129.09, 68.84, 51.26, 42.96, 27.10, 25.89, 24.56, 22.62, 16.16 ppm.

### Example 1.7-2,5-Dioxopyrrolidin-1-yl (1R,5R,8S,9S,E)-5-((benzyl(methyl)carbamoyl)oxy)bicyclo[6.1.0]non-3-ene-9-carboxylate (1.7)

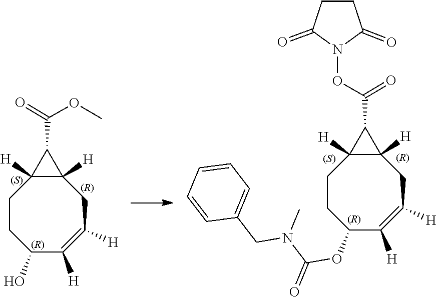

Compound 1.6a (100 mg, 0.51 mmol) was dissolved in methanol (35 mL) and water (2 mL).

KOH (320 mg, 5.70 mmol) was added and the solution was stirred at 30 °C overnight. Citric acid (1.80 g, 9.37 mmol) was added to neutralize the mixture. The reaction mixture was extracted with TBME, washed with water, dried over Na₂SO₄, filtered and concentrated to afford the TCO free carboxylic acid (55 mg as thick oil) in a yield of 60%. The carboxylic acid (30 mg, 0.16 mmol) was dissolved in acetonitrile, di-NHS-carbonate (0.26 g, 1.01 mmol) was added. The reaction mixture was stirred at room temperature for 72 hours. The solvent was evaporated and the residue was purified with silica gel column chromatography (Eluent: 10% EtOAc in PE) to give bis-NHS-TCO (6 mg, 0.014 mmol) in a yield of 9%. The bis-NHS-TCO was dissolved in DCM (5 mL), a solution of N-methylbenzlamine (1.66 mg, 0.014 mmol) in DCM (0.03 mL) was added. The reaction mixture was stirred for 1 hours. The solvent was evaporated at 55 °C. The residue was dissolved in tetrahydrofuran (THF, 5 mL), water (0.2 mL) was added. The solution was stirred at r.t. for 3 days. The mixture was evaporated, dissolved in toluene (6 mL), washed with water (3×1 mL), dried over Na₂SO₄, filtered and concentrated. The residue was purified by silica gel column chromatography (Eluent: from 100% PE to 33% EtOAc in PE) to give the compound 1.7 in a yield of 5 mg (7%). ¹H NMR (400 MHz, CDCl₃) δ 7.4-7.2 (m, 5H), 6.26 (dt, J=16.1, 7.6 Hz, 1H), 5.63 (d, J=17.3, 1H), 5.47 (s, 1H), 4.46 (m, 2H), 2.85 (s, 4H), 2.80 (m, 3H), 2.5-1.0 (m, 9H) ppm. ESI-MS: m/z Calc. for C₂₃H₂₆N₂O₆ 426.18 Da; Obs. [M+H]⁺ 427.17 Da, λₘₐₓ=251 nm.

### Examples 1.8-1.20—Synthesis of 1,3-s-TCO derivatives

### Example 1.8—Ethyl (Z)-bicyclo[6.1.0]non-2-ene-9-carboxylate (1.11)

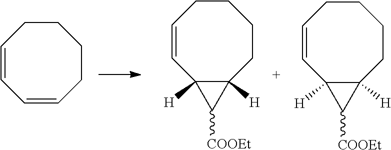

To a mixture of 1,3-cyclooctadiene (5.0 mL, 46.76 mmol) and Rh₂(OAc)₄ (catalytic amount) in DCM (50 mL) at 0 °C, a solution of ethyl diazoacetate (1.0 mL, 9.51 mmol) in DCM (20 mL) was added dropwise for 2 hours. The reaction mixture was stirred at 20 °C for 18 hours. DCM was evaporated in vacuo, and the residue was purified with silica gel column chromatography (Eluent: from 100% heptane to 10% EtOAc in heptane) to give the colorless oil compound 1.11 as a mixture of endo/exo isomers (1 g, 5.1 mmol, 54% yield). ¹H NMR (400 MHz, CDCl₃) δ 5.94-5.65 (m, 1H), 5.48-5.35 (m, 1H), 4.19-4.07 (m, 2H), 2.48-2.30 (m, 1H), 2.16-2.08 (m, 1H), 2.09-1.89 (m, 3H), 1.88-1.73 (m, 1H), 1.71-1.51 (m, 3H), 1.48-1.35 (m, 1H), 1.32-1.22 (m, 2H), 1.15 (t, J=4.7 Hz, 1H), 1.13-1.00 (m, 1H) ppm. ESI-MS: m/z Calc. for C₁₂H₁₈O₂ 194.13 Da; Obs. [M+H]⁺ 195.12 Da.

### Example 1.9—(Z)-Bicyclo[6.1.0]non-2-ene-9-carboxylic acid (1.12)

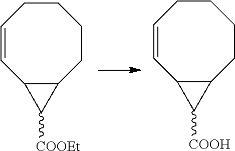 [SMILES: CCOC(=O)C1C2/C=C\CCCCC21 ; O=C(O)C1C2/C=C\CCCCC21]

To a solution of compound 1.11 (1 g, 5.15 mmol) in methanol (20 mL) was added dropwise a solution of NaOH (3.9 g, 98 mmol) in water (30 mL). The reaction mixture was stirred at room temperature for 48 hours under N₂ atmosphere. The methanol was removed in vacuo, and the pH of the residue was adjusted to 2 with HCl solution (2 M). The residue was extracted with EtOAc (3×20 mL) and the combined organic layers were dried over Na₂SO₄ anh., filtered and concentrated. The crude product 1.12 was used directly without further purification as a mixture of endo/exo isomers. ¹H NMR (400 MHz, CDCl₃) δ 5.86-5.63 (m, 1H), 5.51-5.29 (m, 1H), 2.51-2.31 (m, 1H), 2.24-2.15 (m, 1H), 2.12-2.02 (m, 1H), 2.03-1.87 (m, 2H), 1.79-1.55 (m, 3H), 1.49-1.34 (m, 1H), 1.17 (t, J=4.6 Hz, 1H), 1.09 (dtd, J=13.8, 11.8, 3.4 Hz, 1H) ppm. ¹³C NMR (101 MHz, CDCl₃) δ 181.07, 135.50, 123.31, 31.07, 29.94, 29.58, 27.68, 26.70, 26.13, 25.26. ppm. ESI-MS: m/z Calc. for C₁₀H₁₄O₂ 166.10 Da; Obs. [M+H]+ 167.12 Da.

### Example 1.10—(1R,2S,7S,8S,11S)-7-Iodo-9-oxatricyclo[6.3.0.0^{2,11}]undecan-10-one (1.13)

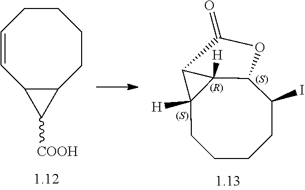

To a solution of compound 1.12 (0.7 g, 4.21 mmol) in DCM (55 mL) was added water (6 mL) and NaHCO₃ (0.7 g, 8.33 mmol). After stirred at room temperature for 30 minutes, a mixture of KI (0.7 g, 4.21 mmol) and 1₂ (1.05 g, 4.13 mmol) was added in small portions within 1 hour to the reaction mixture, and the dark brown mixture was stirred at room temperature for 18 hours under N₂ atmosphere. Subsequently, Na₂SO₃ sat. aq. was added to quench the excess I₂, and the reaction mixture was extracted with EtOAc (3×15 mL). The combined organic layers were dried over Na₂SO₄, filtered and concentrated. The residue was purified by silica gel column chromatography (Eluent: from 20% EtOAc in PE to 50% EtOAc in PE) and gave compound 1.13 in 52% yield (0.62 g, 2.12 mmol). ¹H NMR (400 MHz, CDCl₃) δ 5.03 (t, J=4.1 Hz, 1H), 4.82-4.70 (m, 1H), 2.54-2.40 (m, 1H), 2.29-2.19 (m, 1H), 2.19-1.97 (m, 3H), 1.97-1.83 (m, 2H), 1.71-1.53 (m, 2H), 1.35-1.24 (m, 2H) ppm. ¹³C NMR (101 MHz, CDCl₃) δ 180.43, 82.39, 33.65, 28.75, 24.59, 23.04, 22.66, 22.11, 21.16, 20.97 ppm. ESI-MS: m/z Calc. for C₁₀H₁₃IO₂ 292.00 Da; Obs. [M+H]⁺ 292.96 Da.

### Example 1.11—(1R,2S,8R,11S,Z)-9-Oxatricyclo[6.3.0.0^{2,11}]undec-6-en-10-one (1.14)

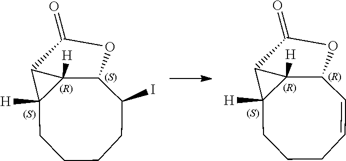

Compound 1.13 (0.50 g, 1.71 mmol) was dissolved in toluene (20 mL), and DBU (0.78 mL, 5.20 mmol) was added. The mixture was heated to 55 °C and allowed to stand for 18 hours under N₂ atmosphere. After cooling the reaction mixture was diluted with brine (30 mL) and extracted with EtOAc (3×25 mL). The combined organic layers were dried over Na₂SO₄, filtered and concentrated. The residue was purified by silica gel column chromatography (Eluent: from 10% EtOAc in PE to 25% EtOAc in PE) to give compound 1.14 (0.15 g, 0.91 mmol) in yield of 54%. ¹H NMR (400 MHz, CDCl₃) δ 5.81-5.54 (m, 2H), 5.27-4.99 (m, 1H), 2.91-2.68 (m, 1H), 2.29 (q, J=6.5 Hz, 1H), 2.11 (ddd, J=8.3, 6.1, 2.0 Hz, 1H), 2.03-1.96 (m, 1H), 1.93-1.83 (m, 1H), 1.72-1.56 (m, 1H), 1.51-1.38 (m, 1H), 1.32-1.12 (m, 2H) ppm. ¹³C NMR (101 MHz, CDCl₃) δ 173.32, 130.77, 125.67, 77.69, 24.20, 24.14, 23.58, 23.15, 21.72, 19.27 ppm. ESI-MS: m/z Calc. for C₁₀H₁₂O₂ 164.08 Da; Obs. [M+H]⁺ 165.00 Da.

### Example 1.12—Methyl (1R,2R,8S,9S,Z)-2-acetoxybicyclo[6.1.0]non-3-ene-9-carboxylate (1.15)

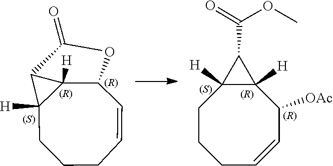

Compound 1.14 (130 mg, 0.80 mmol) was dissolved in methanol (1.5 mL), and KOH (66 mg, 1.18 mmol) was added. The mixture was heated to 55 °C and allowed to stand for 2 hours under N₂ atmosphere. EtOAc (0.4 mL) was added and the mixture was stirred at 55 °C for 20 minutes to quench the excess KOH. After cooling the solvent was removed in vacuo at room temperature, and the residue was dissolved in DMF (4 mL), cooled to 0 °C and iodomethane (0.46 g, 3.80 mmol) was added. The mixture was stirred at room temperature for 2 hours. Subsequently, the reaction mixture was cooled again to 0 °C, pyridine (3 mL), toluene (6 mL), DMAP (8 mg, 0.03 mmol) and acetic anhydride (0.6 mL, 3.80 mmol) were added. The ice bath was removed in 2 hour and the mixture was stirred at room temperature overnight. It was then poured into a mixture of toluene (30 mL), 2M HCl (16 mL) and ice (20 g). The organic layer was washed with water (30 mL). The successive aqueous layers were extracted with toluene (20 mL). The combined organic layers were dried over Na₂SO₄, filtered and concentrated. And the residue was purified by silica gel column chromatography (Eluent: 10% EtOAc in PE) to give compound 1.15 (85 mg, 0.36 mmol) in a yield of 43%. ¹H NMR (500 MHz, CDCl₃) δ 6.20-5.93 (m, 1H), 5.58 (ddd, J=11.0, 3.3, 0.9 Hz, 1H), 5.26-5.06 (m, 1H), 3.58 (s, 3H), 2.36-2.20 (m, 2H), 2.04-1.87 (m, 2H), 1.95 (s, 3H), 1.73-1.64 (m, 1H), 1.65 (t, J=9.1 Hz, 1H), 1.54-1.45 (m, 2H), 1.40-1.23 (m, 1H) ppm. ¹³C NMR (126 MHz, CDCl₃) δ 171.88, 170.57, 133.48, 125.53, 71.05, 51.30, 28.94, 25.95, 24.79, 24.78, 21.15, 20.24, 17.79 ppm. ESI-MS: m/z Calc. for C₁₃H₁₈O₄ 238.12 Da; Obs. [M+H]⁺ 239.08 Da.

### Example 1.13—Methyl (1R,2R,8S,9S,E)-2-hydroxybicyclo[6.1.0]non-3-ene-9-carboxylate (1.16a, 1.16b; 2 isomers with the allylic release-OH either in axial or equatorial position)

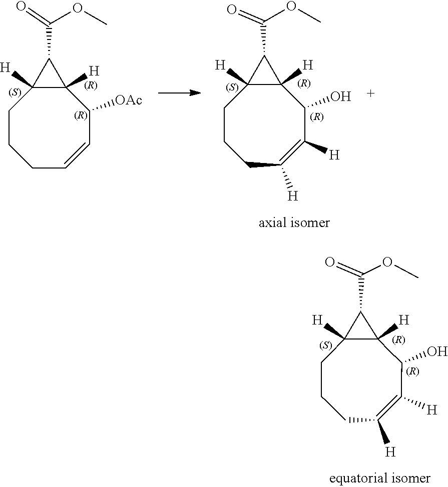

Axial isomer compound 1.16a (5 mg, 0.02 mmol, in 8% yield) and equatorial isomer compound 1.16b (7 mg, 0.03 mmol, in 12% yield) were obtained from compound 1.15 using General Procedure A.

### Axial Isomer (1.16a)

¹H NMR (399 MHz, CDCl₃) δ 5.96 (ddt, J=17.2, 7.6, 1.4, 1H), 5.70 (dd, J=7.5, 2.6 Hz, 1H), 5.57 (ddd, J=17.2, 2.6, 1.3 Hz, 1H), 3.68 (s, 3H), 2.40-2.22 (m, 2H), 2.15-1.97 (m, 2H), 2.04 (s, 3H), 1.90 (m, 2H), 1.75 (ddd, J=10.9, 9.1, 7.4 Hz, 1H), 1.65 (m, 1H), 1.13 (dtd, J=11.5, 9.0, 5.6 Hz, 1H) ppm.

### Equatorial Isomer (1.16b)

¹H NMR (399 MHz, CDCl₃) δ 6.22 (dd, J=17.0, 8.5 Hz, 1H), 5.92 (td, J=8.5, 1.4 Hz, 1H), 5.59 (dddd, J=16.9, 11.3, 4.1, 1.4 Hz, 1H), 3.70 (s, 3H), 2.41 (m, 2H), 2.02 (s, 3H), 2.00 (m, 2H), 1.92 (m, 2H), 1.81 (dd, J=10.0, 9.1 Hz, 1H), 1.24 (m, 2H) ppm.

### Example 1.14—(1R,2S,8R,11S,E)-9-Oxatricyclo[6.3.0.0^{2,11}]undec-6-en-10-one (1.17a, 1.17b)

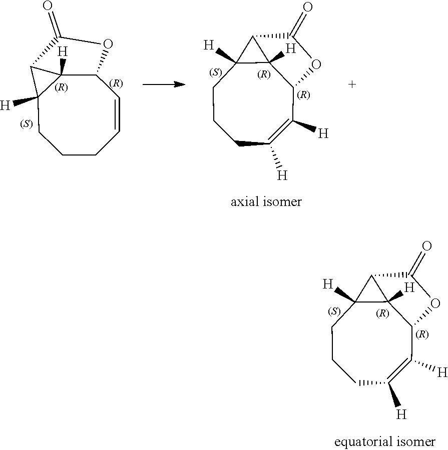

Axial isomer compound 1.17a (155 mg, 0.94 mmol, in 33% yield) and equatorial isomer compound 1.17b (100 mg, 0.61 mmol, in 22% yield) were obtained from compound 1.14 (460 mg, 2.80 mmol) using General Procedure A.

### Axial Isomer (1.17a)

¹H NMR (400 MHz, CDCl₃): δ 6.00 (dddd, J=17.1, 9.7. 6.6, 1.0 Hz, 1H), 5.62 (dt. J=17.1, 1.0 Hz, 1H), 5.29 (d, J=5.0 Hz, 1H), 2.43 (m, 2H), 2.24 (dd, J=9.1, 6.4, 1H), 2.11 (m, 2H), 2.01 (m, 1H), 1.73 (m, 1H), 1.42 (m, 1H), 1.31 (m, 1H) ppm. ¹³C NMR (101 MHz, CDCl₃) δ 175.06, 131.65, 131.07, 77.53, 32.76, 32.55, 25.32, 24.96, 23.78, 20.90 ppm.

### Equatorial Isomer (1.17b)

¹H NMR (400 MHz, CDCl₃): δ 5.68 (m, 2H), 5.44 (dd, J=5.6, 1.8 Hz, 1H), 2.81 (ddd, 7.4, 6.4, 5.8 Hz, 1H), 2.63 (m, 1H), 2.29 (m, 1H), 2.28 (dd, J=9.3, 6.5 Hz, 1H), 2.07 (m, 1H), 1.98 (m, 1H), 1.43 (m, 1H), 1.15 (m, 1H), 0.82 (m, 1H) ppm. ¹³C NMR (101 MHz, CDCl₃) δ ¹³C NMR (101 MHz, CDCl₃) δ 174.24, 139.84, 134.60, 78.28, 36.74, 34.94, 27.40, 27.34, 27.31, 25.93 ppm.

### Example 1.15—(1R,2R,8S,9S,E)-2-Hydroxybicyclo[6.1.0]non-3-ene-9-carboxylic acid, potassium salt (1.18a)

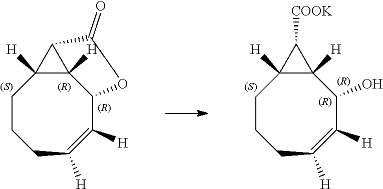

Compound 1.17a (19.4 mg, 0.118 mmol) was dissolved in methanol (0.6 mL), and KOH (10 mg, 0.177 mmol) was added. The solution was stirred at 45 °C for 1 hour under Ar atmosphere. EtOAc (0.5 mL) was added, and the solution was stirred at 45 °C for an additional hour to quench the excess KOH. Subsequently, the solvent was removed in vacuo, and the residue was stirred in acetonitrile (20 mL). The turbid mixture was filtered, and the filtrate was evaporated to dryness to yield 17.4 mg of product as its potassium salt (67% yield). ¹H NMR (400 MHz, MeOD-d4): δ 5.94 (dddd, J=17.0, 8.1, 6.9, 1.1 Hz, 1H), 5.72 (ddd, J=17.0, 2.5, 1.2 Hz, 1H), 4.82 (dd, J=8.1, 2.4 Hz, 1H), 2.35 (m, 1H), 2.10-1.80 (m, 4H), 1.73 (m, 2H), 1.47 (td, J=9.1, 7.9 Hz, 1H), 1.04 (dddd, J=11.0, 10.1, 9.0, 5.1 Hz, 1H) ppm. ¹³C NMR (101 MHz, MeOD-d4) δ 179.03, 139.31, 124.95, 67.58, 31.56, 29.84, 29.46, 24.66, 21.93, 21.15 ppm.

### Example 1.16—(1S,2S,8R,9R,E)-2-Hydroxybicyclo[6.1.0]non-3-ene-9-carboxylic acid, potassium salt (1.18b)

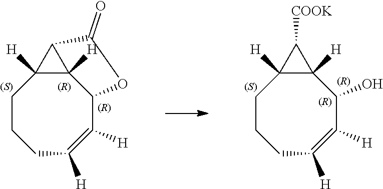

Compound 1.17b (19.5 mg, 0.119 mmol) was dissolved in methanol (0.6 mL), and KOH (10 mg, 0.177 mmol) was added. The solution was stirred at 45 °C for 2 hour under Ar atmosphere. EtOAc (0.5 mL) was added, and the solution was stirred at 45 °C for an additional hour to quench the excess KOH. Subsequently, the solvent was removed in vacuo, and the residue was stirred in acetonitrile (20 mL). The turbid mixture was filtered, and the filtrate was evaporated to dryness to yield 15.3 mg of product 1.18b as its potassium salt (58% yield). ¹H NMR (400 MHz, MeOD-d4): δ 6.01 (dd, J=16.9, 7.8 Hz, 1H), 5.44 (dddd, J=16.9, 11.3, 4.0, 1.6 Hz, 1H), 4.91 (ddd, J=9.4, 7.8, 1.5 Hz, 1H), 2.35 (dtd, J=10.5, 4.0, 1.6 Hz, 1H), 2.17 (ddd, J=15.1, 7.2, 4.5 Hz, 1H), 1.97 (t, J=8.8 Hz, 1H), 1.93 (m, 1H), 1.87 (m, 1H), 1.78 (qd, J=11.5, 4.8 Hz, 1H), 1.34 (dddd, J=13.5, 12.0, 10.9, 4.3 Hz, 1H), 1.18 (dt, J=15.1, 11.4 Hz, 1H), 0.89 (dddd, J=11.7, 10.4, 8.4, 4.6 Hz, 1H) ppm. ¹³C NMR (101 MHz, MeOD-d4) δ 179.14, 140.18, 129.45, 70.61, 38.36, 33.94, 29.55, 28.76, 28.04, 24.83 ppm.

### Example 1.17—Methyl (1R,2R,8S,9S,E)-2-hydroxybicyclo[6.1.0]non-3-ene-9-carboxylate (1.19a)

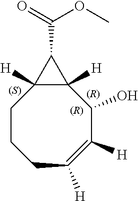

Compound 1.17a (9.75 mg, 0.0595 mmol) was dissolved in methanol (0.6 mL), and NaOMe (0.040 mL 25% in methanol, 0.185 mmol) was added. The solution was stirred at 50 °C for 24 hours under Ar atmosphere, and then neutralized by addition of IR120-H acidic ion-exchange resin (100 mg), filtered, and evaporated to dryness to yield the product as a colorless oil in a yield of 11 mg (95%). ¹H NMR (400 MHz, MeOD-d4): δ 5.95 (dddd, J=17.1, 8.0, 6.9, 1.1 Hz, 1H), 5.73 (ddd, J=17.0, 2.5, 1.2 Hz, 1H), 4.82 (dd, J=8.1, 2.4 Hz, 1H), 3.67 (s, 3H), 2.33 (m, 1H), 2.00 (m, 1H), 1.96 (dd, J=10.2, 9.2 Hz, 1H), 1.87 (m, 2H), 1.75 (m, 2H), 1.47 (td, J=9.1, 8.1 Hz, 1H), 1.04 (dddd, J=11.0, 10.1, 9.0, 5.2 Hz, 1H) ppm. ¹³C NMR (101 MHz, CDCl₃) δ 175.13, 131.63, 131.09, 77.56, 52.22, 32.77, 32.55, 25.32, 24.97, 23.79, 20.90 ppm.

### Example 1.18—2,5-Dioxopyrrolidin-1-yl (1R,2R,8S,9S,E)-2-((((2,5-dioxopyrrolidin-1-yl)oxy)carbonyl)oxy) bicyclo[6.1.0]non-3-ene-9-carboxylate (1.20)

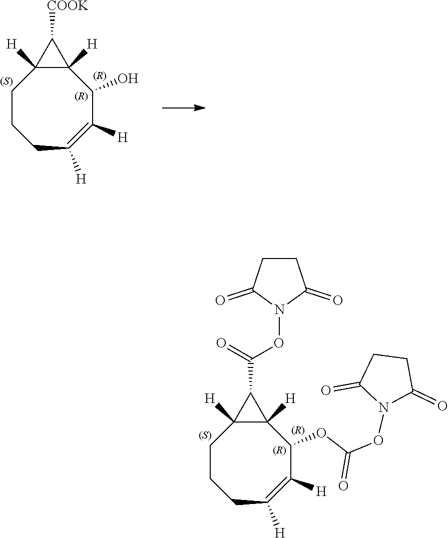

Compound 1.18a (13 mg, 0.059 mmol) was dissolved in dimethylsulfoxide (DMSO, 0.6 mL), and di-NHS-carbonate (75 mg, 0.296 mmol) was added. The clear mixture was stirred at room temperature for 15 minutes to yield the desired compound 1.20, which was used without further purification. ESI-MS: m/z Calc. for C₁₉H₂₀N₂O₉ 420.12 Da; Obs. [M+Na]⁺ 443.17 Da.

### Example 1.19—2,5-Dioxopyrrolidin-1-yl (1R,2R,8S,9S,E)-2-((benzyl(methyl)carbamoyl)oxy)bicyclo[6.1.0]non-3-ene-9-carboxylate (1.21)

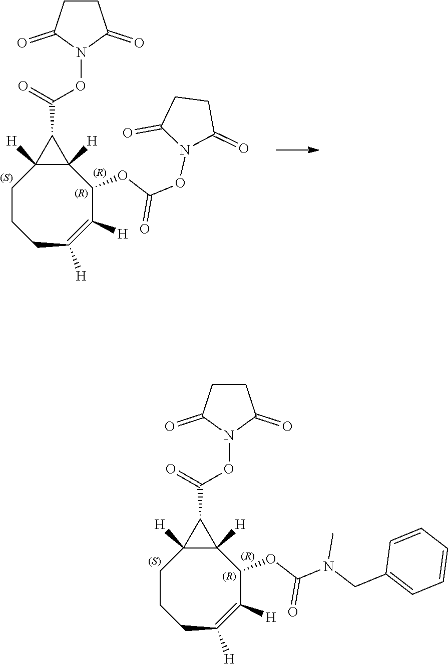

Compound 1.20 (0.059 mmol) in DMSO (0.6 mL) was added N-methylbenzylamine (36 mg, 0.30 mmol), and the clear mixture was stirred at room temperature for 45 minutes. Then, the solution was diluted with acetonitrile (0.6 mL), formic acid (0.04 mL), and water (1.2 mL), and purified by RP preparative HPLC (50% acetonitrile in water), and subsequently freeze-dried, to yield the product as a white powder (6.0 mg, 24%). ¹H NMR (400 MHz, CDCl₃, mixture of two conformers): δ 7.35-7.15 (m, 5H), 6.02 (m, 1H), 5.82 (m, 1H), 5.65 (dd, J=17.4, 4.1 Hz, 1H), 4.85 (d, J=15.2 Hz, 0.53H), 4.82 (d, J=15.5 Hz, 0.47H), 4.20 (d, J=15.3 Hz, 0.53H), 3.84 (d, J=15.5 Hz, 0.47H), 2.84 (s, 1.88H), 2.80 (s, 2.12H), 2.72 (s, 3H), 2.35 (m, 1H), 2.25 (t, J=9.6 Hz, 1H), 2.10 (m, 2H), 1.94 (m, 3H), 1.71 (m, 1H), 1.54 (m, 1H) ppm. ESI-MS: m/z Calc. for C₂₃H₂₆N₂O₆ 426.18 Da; Obs. [M+Na]⁺ 449.33 Da.

### Example 1.20—Doxorubicin-1,3-s-TCO-NHS (1.22)

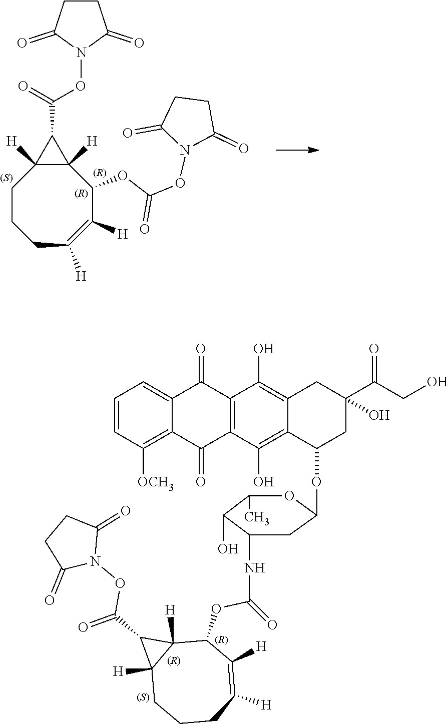

To a solution of compound 1.20 (0.050 mmol) in DMSO (0.6 mL) was added DIPEA (32 mg, 0.25 mmol), and doxorubicin hydrochloride (66 mg, 0.114 mmol). The dark orange and clear solution was stirred at room temperature for 3 hours, and then diluted with acetonitrile (1.2 mL), formic acid (0.05 mL), and water (1.8 mL). The turbid mixture was centrifuged and filtered, and purified by RP preparative HPLC (50% acetonitrile in water), and subsequently freeze-dried, to yield the product as an orange powder (2.5 mg mixture of diastereomers, 6%). ESI-MS: m/z Calc. for C₄₂H₄₄N₂O₁₇ 848.26 Da; Obs. [M+Na]⁺ 871.50 Da, [M−H]⁻ 847.42 Da.

### Examples 1.21-1.24 Synthesis of exo 1,3-s-TCO and 1,5-s-TCO derivatives

### Example 1.21—Methyl (1R,2R,8S,9R,Z)-2-hydroxybicyclo[6.1.0]non-3-ene-9-carboxylate (1.23)

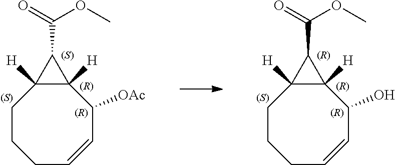

To a solution of compound 1.15 (1 eq) in dry tetrahydrofuran (THF) (0.1M of compound 1.15 in THF) is added a solution of 1M potassium tert-butoxide in THE (1.2 eq). Alternative solvents to be used in addition to or instead of THE are ethanol and ether, which may be dry or wet; preferably a mixture of THE and dry ethanol is used, or wet ether. The reaction mixture is stirred at room temperature overnight. Solvent is evaporated in vacuo, and the residue is purified with silica gel column chromatography (Eluent: 20% EtOAc in heptane) to give compound 1.23.

### Example 1.22—Methyl (1R,2R,8S,9R,E)-2-hydroxybicyclo[6.1.0]non-3-ene-9-carboxylate (1.24a, 1.24b; 2 isomers with the allylic release-OH either in axial or equatorial position)

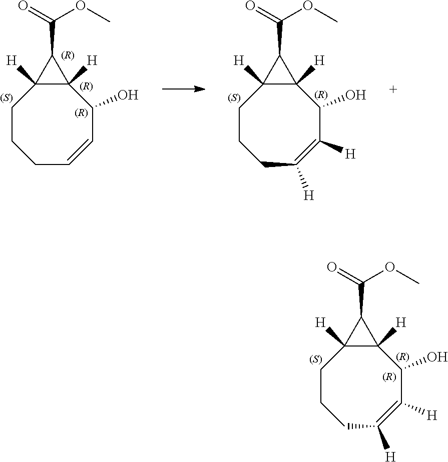

Axial isomer compound 1.24a and equatorial isomer compound 1.24b are obtained from compound 1.23 using General Procedure A.

### Example 1.23—Methyl (1R,5R,8S,9R,Z)-5-hydroxybicyclo[6.1.0]non-3-ene-9-carboxylate (1.25)

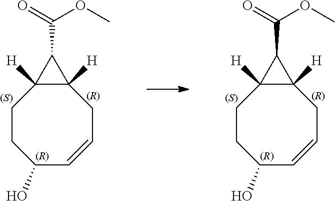

To a solution of compound 1.5 (1 eq) in dry THF (0.1M of compound 1.5 in THF) is added a solution of 1M potassium tert-butoxide in THE (1.2 eq). The reaction mixture is stirred at room temperature overnight. Solvent is evaporated in vacuo, and the residue is purified with silica gel column chromatography (Eluent: 20% EtOAc in heptane) to give compound 1.25.

### Example 1.24—Methyl (1R,5R,8S,9R,E)-5-hydroxybicyclo[6.1.0]non-3-ene-9-carboxylate (1.26a, 1.26b; 2 isomers with the allylic release-OH either in axial or equatorial position)

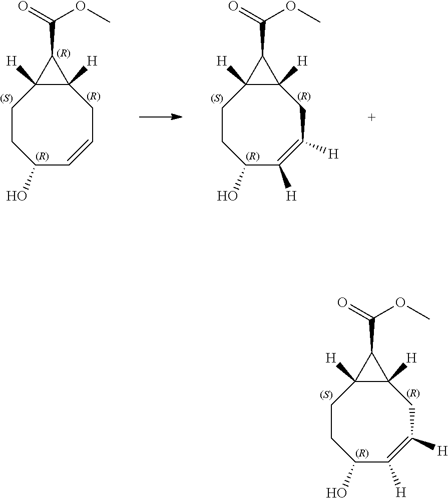

Axial isomer compound 1.26a and equatorial isomer compound 1.26b is obtained from compound 1.25 using General Procedure A.

### Example 2: Reactivity Measurements

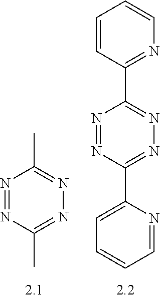

The second-order rate constant of the IEDDA reaction of 3,6-dimethyl-1,2,4,5-tetrazine (2.1) or 3,6-di(pyridin-2-yl)-1,2,4,5-tetrazine (2.2) and a compound of the disclosure in MeCN at 20 °C was determined by UV-Vis spectrometry under second-order reaction conditions (n=1). A cuvette was filled with a solution of 3,6-dimethyl-1,2,4,5-tetrazine or 3,6-di(pyridin-2-yl)-1,2,4,5-tetrazine (3 mL 0.083 mM in MeCN), and equilibrated at 20 °C. Next, a solution of the compound of the disclosure (10.0 μL 25 mM in acetonitrile, 2.50×10⁻⁷ mol) was added, and the solution was quickly homogenized and inserted in the UV spectrometer. The absorption at λ=540 nm (specific for the tetrazine moiety) was measured every second in a time course experiment. From the decay of this absorption, the conversion was calculated. The second-order rate constant k₂ was determined from the slope of the curve of a plot of 1/c −1/c₀ versus time.

TABLE 1
Rate constants k₂ (M⁻¹ s⁻¹) measured for the
reaction between compounds of the disclosure (TCO)
and derivatives with different activator in MeCN at 20 °C.

| Compound | Activator | k₂ (M⁻¹ s⁻¹) |
|---|---|---|
| 1.6a | 2.1 | 15 |
| | 2.2 | 1500 |
| 1.6b | 2.1 | 3.3 |
| 1.7 | 2.1 | 2.9 |
| | 2.2 | 375 |
| 1.16a | 2.1 | 7.0 |
| 1.16b | 2.1 | 0.3 |
| 1.17a | 2.1 | 11.5 |
| | 2.2 | 1100 |
| 1.17b | 2.1 | 8.7 |
| 1.18a | 2.1 | 35 |
| 1.18b | 2.1 | 4 |
| 1.21 | 2.1 | 8.0 |
| | 2.2 | 605 |

### Example 3: Releasing Activity of s-TCO Derivatives

### Example 3.1—Reaction of 1.7 and 3,6-dimethyl-1,2,4,5-tetrazine

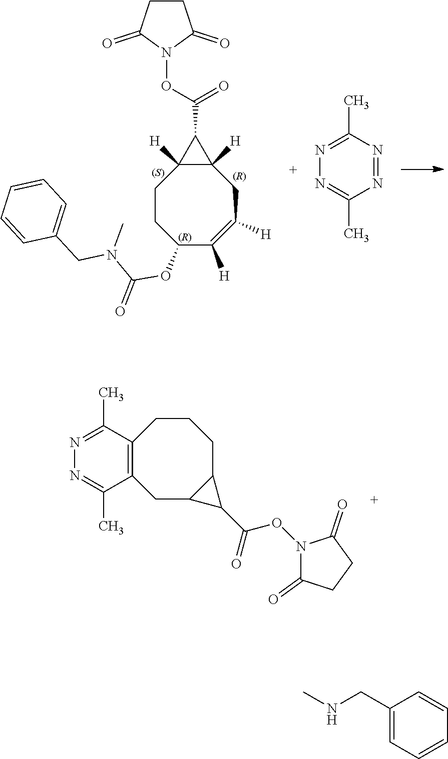

To a stock solution of 1.7 (10 uL 17.8 mM in DMSO, 1.78×10⁻⁷ mol) was added acetonitrile (0.2 mL), PBS (0.8 mL), and a stock solution of 3,6-dimethyl-1,2,4,5-tetrazine (7.1 uL 25 mM in DMSO, 1.78×10⁻⁷ mol). The mixture was homogenized and the pink color disappeared within minutes. After 30 min, HPLC-MS PDA analysis showed the quantitative formation of the pyridazine elimination product without the N-methylbenzylamine. ESI-MS: m/z Calc. for C₁₈H₂₁N₃O₄ 343.15 Da; Obs. [M+H]⁺ 344.25 Da, max=243 nm.

### Example 3.2—Reaction of 1.21 and 3,6-dimethyl-1,2,4,5-tetrazine

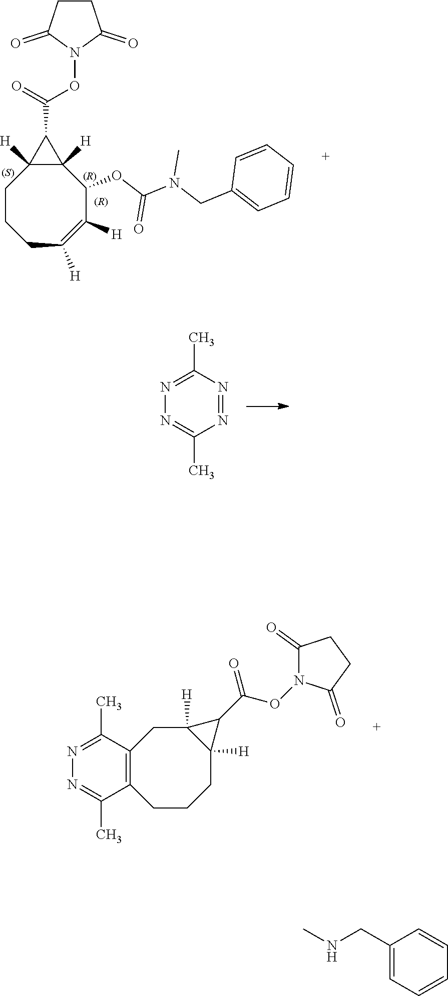

To a solution of 1.21 (3.75×10⁻⁷ mol) in acetonitrile (0.2 mL) was added PBS (0.8 mL), and a stock solution of 3,6-dimethyl-1,2,4,5-tetrazine (20 uL 25 mM in DMSO, 5.0×10⁻⁷ mol). The mixture was homogenized and the pink color disappeared within minutes. After 30 min, HPLC-MS PDA analysis showed the quantitative formation of the elimination product. ESI-MS: m/z Calc. for C₁₈H₂₁N₃O₄ 343.15 Da; Obs. [M+H]⁺ 344.25 Da, λₘₐₓ=250 nm.

### Example 3.3—Reaction of 1.22 and 3,6-dimethyl-1,2,4,5-tetrazine

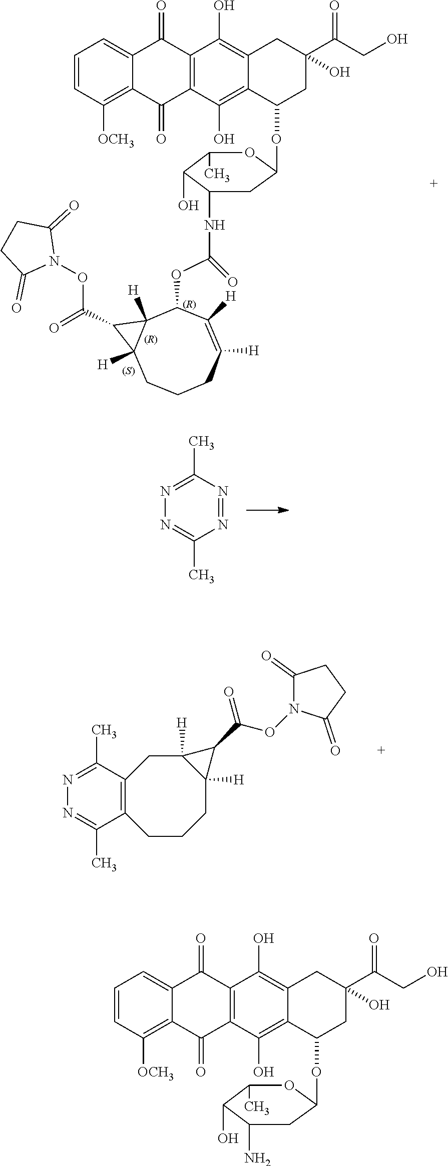

To a solution of 1.22 (3.75×10-8 mol) in acetonitrile (0.02 mL) was added PBS (0.08 mL), and a stock solution of 3,6-dimethyl-1,2,4,5-tetrazine (2 uL 25 mM in DMSO, 5.0×10-8 mol). The mixture was homogenized. and after 30 min HPLC-MS PDA analysis showed the quantitative formation of the elimination product (ESI-MS: m/z Calc. for C₁₈H₂₁N₃O₄ 343.15 Da; Obs. [M+H]⁺ 344.25 Da, Xmax=257 nm) and doxorubicine (ESI-MS: m/z Calc. for C₂₇H₂₉NO₁₁ 543.17 Da; Obs. [M+H]⁺ 544.25, [M−H]⁻ Da 542.33, Xmax=479 nm).

### Example 4: Antibody Conjugation and Evaluation

### Example 4.1—Antibody Conjugation of CC49-1,5-sTCO-Benzylamine (1.8)

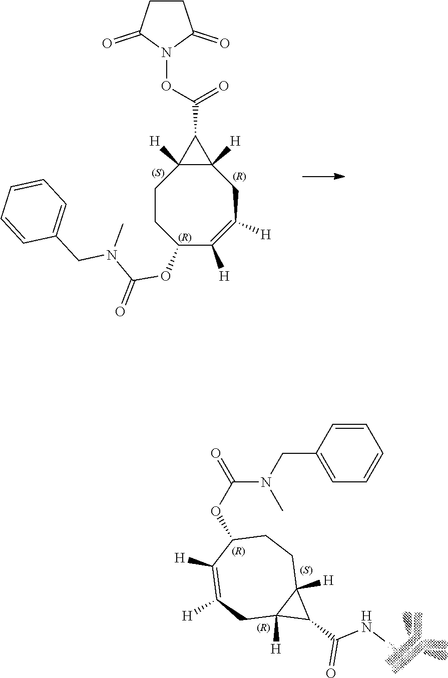

Compound 1.7 was dissolved in dry DMF at a 20 mM concentration. An anti-TAG72 IgG (CC49) was reacted with 40 eq 1.7 in a PBS/propylene glycol/DMF 70:20:10 mixture. The pH was adjusted to 8.5 with 1M sodium carbonate and the reaction mixture was incubated at room temperature in the dark on a roller bench. After 2 h incubation, the conjugation product 1.8 was purified by PD-10 pre-equilibrated with 25% propylene glycol in PBS. SEC and SDS-PAGE analysis of compound 1.8 showed >95% purity while a tetrazine titration showed the presence of an average 3.1 tags per IgG. After 16 months mAb storage in PBS at +4 °C in the dark 78% of the CC49-conjugated TCO was found to be still reactive towards tetrazines, confirming the relative stability of the s-TCO in storage conditions.

### Example 4.2—Kinetic Measurements

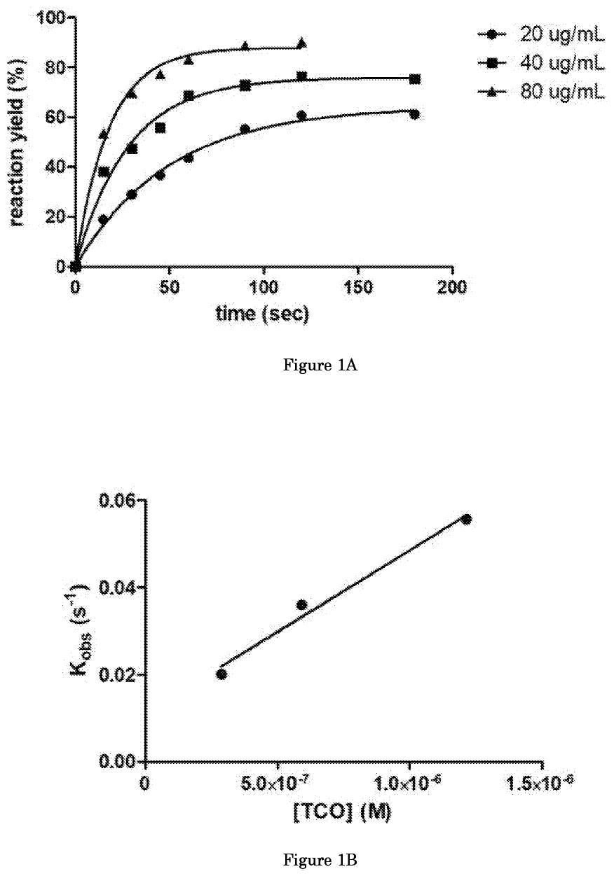

Kinetic measurements between 1.8 and an ¹¹¹In-labeled tetrazine were carried out in pseudo-first order conditions, as previously described (Bioconjugate Chem. 2016, 27, 1697-1706). Briefly, various amounts of mAb conjugate (20, 40 and 80 μg/mL) were reacted with the radiolabeled tetrazine at 37 °C in PBS. Aliquots of the reaction mixtures (20 μL) taken at various time points between 15 sec and 3 min were quenched with a large excess of non-radioactive tetrazine and then analyzed by SDS-PAGE and phosphor imager. The cycloaddition yields were determined from the % radioactivity in the bands corresponding to the mAb. The reaction yields with time were fitted to a first-order exponential (FIG. 1A) and a pseudo first-order rate constant (k_{obs}) was determined from the fit. The values of k_{obs} were then plotted vs. the concentration of TCO and fitted using linear regression (FIG. 1B). Based on k_{obs}=[TCO](k₂), a (49.6±5.1)×10³ M⁻¹s⁻¹ second order rate constant was calculated from the slope of the line.

### Example 4.3—Methyl Benzylamine Release from CC49 Conjugate 1.8

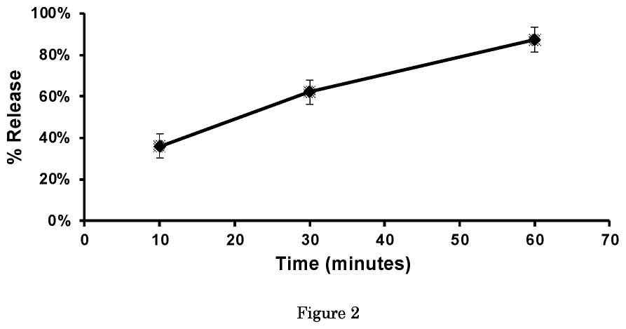

30 μL of CC49-1,5-s-TCO-MBA (1.8) solution (2.56 μg/pL in 25% PG in PBS) was transferred to a 0.5 mL Eppendorf tube, and 0.6 μL 3,6-dimethyl-1,2,4,5-tetrazine 2.1 solution (25 mM in DMSO) was added. The solution was homogenized and incubated at 37 °C for a specific time. Then, 150 μL of ice-cold acetonitrile was added, and the mixture was vortexed, stored at −18 °C for 30 min, and centrifuged for 5 min at 13000 rpm. 100 μL of supernatant was taken and transferred to a sample vial. HPLC-SIM-MS analysis was used to quantify the concentration of N-methylbenzylamine (m/z=+122 Da) using a calibration curve, and the release yield was determined. All time points were performed in triplicate (N=3). 36.1% of releasable N-methylbenzylamine was released at the first recorded timepoint (10 min). There was 62.2% and 87.3% release at the subsequent 30 and 60 minutes timepoints demonstrating efficient payload release. The results of this experiment are shown in FIG. 2.

### Example 4.4—Blood Circulation and In Vivo Stability

The conjugate 1.8 was labeled with iodine-125 using the Bolton-Hunter method, as previously described (Bioconjugate Chem. 2016, 27, 1697-1706). Female tumor-free nude mice (n=3) were injected 200 μg ¹²⁵I-labeled 1.8 (ca. 0.5 MBq/mouse in 100 μL PBS). Blood samples (ca. 40 μL) were withdrawn from the vena saphena at 1 and 6 hours, 1, 2 and 3 days followed by one last collection via heart puncture at euthanasia. The blood samples were immediately measured in a gamma counter together with standards to determine the % injected dose per gram (% ID/g) and then they were stored at −20 °C. The radioactivity levels in blood showed a two-phase elimination profile typical of intact IgGs with a 2.8 h t_{1/2,α} (54.3%) and a 64.4 h t_{1/2,β}.

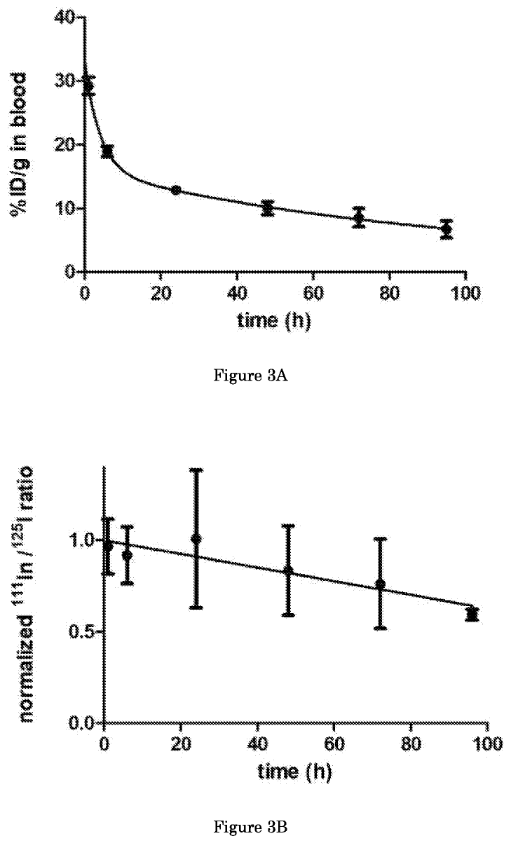

At the end of the experiment, all blood samples were thawed, diluted to 100 μL with PBS and reacted ex vivo with an excess of ¹¹¹In-labeled tetrazine (labeled at 0.15 MBq/μg). After 1 h incubation at 37 °C, the reaction mixtures were centrifuged at 12.5 krpm for 5 min and the supernatants were purified twice via Zeba desalting spin columns (0.5 mL, 40 kDa MW cut-off) pre-equilibrated with PBS, to remove unreacted tetrazine. The eluates were then measured in a gamma-counter with a dual-isotope protocol with cross-contamination correction. As in previous studies (Bioconjugate Chem. 2016, 27, 1697-1706), the In-111/I-125 cpm ratio in the samples was plotted vs time (FIG. 3A). The in vivo TCO deactivation half-life (5.6 days) was calculated from the linear fitting of the data (FIG. 3B).

This shows that the compounds of the disclosure have an excellent stability in vivo.

1. A compound, or a salt, solvate, hydrate, and/or an enantiomer thereof, wherein the compound comprises an (E)-bicyclo[6.1.0]non-3-ene moiety, wherein at least one allylic carbon of said moiety is in the R-configuration and is substituted with R₄₈;
R₄₈ is selected from the group consisting of —OH, —O-acetyl, —O—C₁₋₄ alkyl, halogen, active carbonate, and a releasable group;
the carbon atom at position 1 of said moiety is in the R-configuration;
the carbon atom at position 8 of said moiety is in the S-configuration; and
preferably the carbon atom at position 9 of said moiety is substituted.

2. The compound according to claim 1, or the salt, solvate, hydrate, and/or enantiomer thereof, wherein said compound has a structure according to any one of Formulae (Ia) and (Ib): 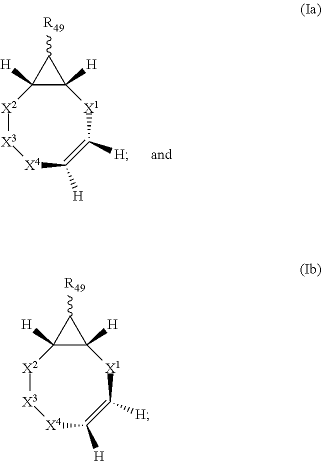
wherein
each of X, X², X³, and X⁴ is independently a substituted or unsubstituted carbon;
at least one of X¹ and X⁴ is —CHR₄₈;
R₄₉ is selected from the group consisting of —C(O)OH, —C(O)O—CH₃, —C(O)NH₂, active esters, and —(S^{P})_{D}—C^{B};
S^{P} is a spacer;
D is 0 or 1, and
C^{B} is a construct B, which is an organic molecule or an inorganic molecule;
preferably one of X¹ and X⁴ is —CHR₄₈; and
preferably D is 1.

3. The compound according to any one of the preceding claims, or the salt, solvate, hydrate, and/or enantiomer thereof, wherein said compound has a structure according to any one of Formulae (II-EQ15), (II-AX15), (II-EQ13), and (II-AX13):

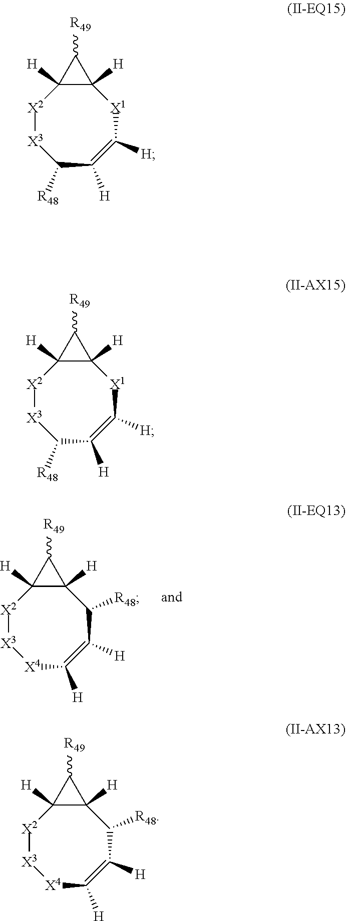

4. The compound according to any one of the preceding claims, or the salt, solvate, hydrate, and/or enantiomer thereof, wherein said compound has a structure according to any one of Formulae (IV-EQ15EN), (IV-AX15EN), (IV-EQ13EN), (IV-AX13EN), (IV-EQ15EX), (IV-AX15EX), (IV-EQ13EX), and (IV-AX13EX):

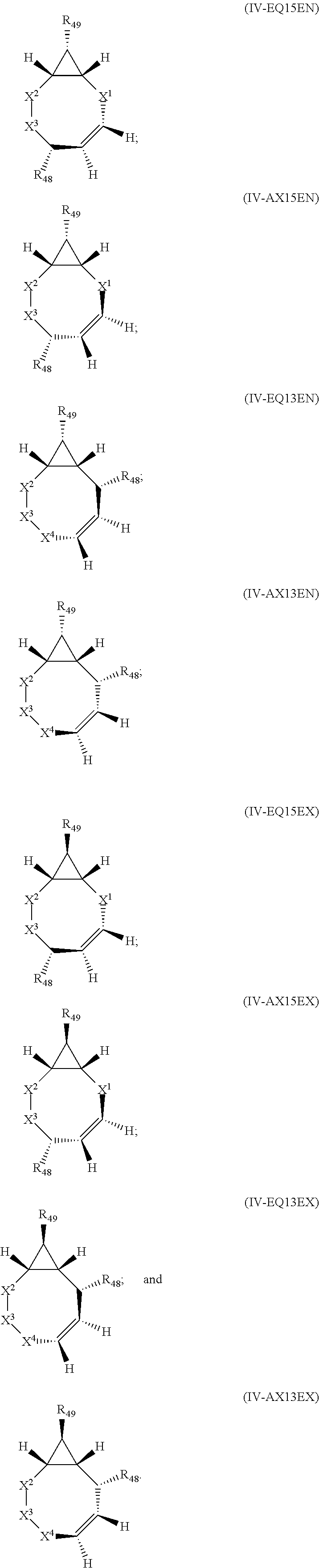

5. The compound according to any one of claims 2 to 4, or the salt, solvate, hydrate, and/or enantiomer thereof, wherein each of X¹, X², X³, and X⁴ is independently C(R₄₇)₂; provided that in Formulae (Ia) and (Ib) at least one of X¹ and X⁴ is —CHR₄₈;
each R₄₇ is independently selected from the group consisting of hydrogen, halogen, (hetero)(cyclo)alkyl, (hetero)(cyclo)alkenyl, (hetero)(cyclo)alkynyl, (hetero)aryl, —(S^{P})_{D}—C^{B}, and combinations thereof:
wherein the (hetero)(cyclo)alkyl, (hetero)(cyclo)alkenyl, (hetero)(cyclo)alkynyl, and (hetero)aryl groups are optionally substituted; and
preferably at most two R₄₇ are not hydrogen,
more preferably at most one R₄₇ is not hydrogen; and
most preferably all R₄₇ are hydrogen.

6. The compound according to any one of claims 2 to 5, or the salt, solvate, hydrate, and/or enantiomer thereof, wherein:
(a) R₄₈ is —OH and R₄₉ is —COOH;
(b) R₄₈ is an active carbonate and R₄₉ is an active ester;
(c) R₄₈ is a releasable group and R₄₉ is an active ester; or
(d) R₄₈ is a releasable group and R₄₉ is —(S^{P})_{D}—C^{B}.

7. The compound according to any one of the preceding claims, or the salt, solvate, hydrate, and/or enantiomer thereof, wherein the releasable group is —(Y¹—C(═Y²))ᵢ—(S^{P})ⱼC^{A}; wherein each of Y¹ and Y² are independently selected from 0, and S; preferably Y¹ and Y² are O;
C^{A} is Construct A, which is a payload;
j is 0 or 1;
i is 0 or 1;
if i is 0, —(S^{P})ⱼ—C^{A} is connected to the remainder of the compound via 0 or S, that is part of —(S^{P})ⱼ—C^{A};
if i is 1, —(S^{P})ⱼ—C^{A} is connected to —C(═Y²)—via O, S, secondary N, or a tertiary N, that is part of —(S^{P})ⱼ—C^{A};
preferably C^{A} is an organic molecule or an inorganic molecule;
preferably j is 0; and
preferably i is 1.

8. The compound according to claim 7, or the salt, solvate, hydrate, and/or enantiomer thereof, wherein C^{A} is selected from the group consisting of a drug, a nucleic acid, a peptide, a protein, a carbohydrate, an aptamer, a hormone, a toxin, a steroid, a cytokine, a lipid, a small organic molecule, a polymer, LNA, PNA, an amino acid, a peptoid, a chelating moiety, a molecule comprising a radionuclide, a fluorescent dye, a phosphorescent dye, a resin, a bead, an organic particle, a gel, an organic surface, an organometallic compound, a cell, an inorganic surface, an inorganic particle, an allotrope of carbon, an inorganic drug, a radionuclide, and combinations thereof, and
preferably C^{A} is a drug;
more preferably C^{A} is a cancer drug.

9. The compound according to any one of claims 2 to 8, or the salt, solvate, hydrate, and/or enantiomer thereof, wherein C^{B} is selected from the group consisting of a nucleic acid, a peptide, a protein, a carbohydrate, an aptamer, a hormone, a toxin, a steroid, a cytokine, a lipid, a small organic molecule, a polymer, LNA, PNA, an amino acid, a peptoid, a chelating moiety, a molecule comprising a radionuclide, a fluorescent dye, a phosphorescent dye, a drug, a resin, a bead, an organic particle, a gel, an organic surface, an organometallic compound, a cell, an inorganic surface, an inorganic particle, an allotrope of carbon, an inorganic drug, a radionuclide, and combinations thereof, and
preferably C^{B} is a protein or a polymer;
more preferably C^{B} is an antibody or polyethylene glycol.

10. A composition comprising:
(a) a compound according to any one of the preceding claims, or the salt, solvate, or hydrate thereof, and
(b) the enantiomer of said compound, or the salt, solvate, or hydrate thereof, and
preferably said composition is a racemic mixture of (a) and (b).

11. A combination of
(A1) a compound according to any one of the preceding claims, or the salt, solvate, hydrate, and/or enantiomer thereof; or
(A2) a composition according to claim 10: with
(B) a diene or a salt, solvate, or hydrate thereof;
preferably the diene is a tetrazine.

12. The compound according to any one of claims 1 to 9, or the salt, solvate, hydrate, and/or enantiomer thereof, the composition according to claim 10; or the combination according to claim 11; for use as a medicament.

13. The compound according to any one of claims 1 to 9, or the salt, solvate, hydrate, and/or enantiomer thereof, the composition according to claim 10; or the combination according to claim 11; for use in the treatment of a disease in a subject,
preferably the subject is a human,
preferably the disease is cancer.

14. A non-therapeutic method for reacting:
(ia) a compound according to any one of claims 1 to 9, or a salt, solvate, hydrate, and/or an enantiomer thereof, or
(iia) a composition according to claim 10;
with a diene or a salt, solvate, or hydrate thereof,
wherein said method comprises the step of contacting (ia) or (iia) with said diene or salt, solvate, or hydrate thereof,
preferably said contacting is in vitro; and
preferably said diene is a tetrazine.

15. An intermediate, or a salt, solvate, hydrate, and/or an enantiomer thereof, selected from the group consisting of Formulae (INT15-1), (INT15-2), (INT15-3), (INT13-1), (INT13-2), (INT13-3), (INTAX13-4), and (INTEQ13-4): 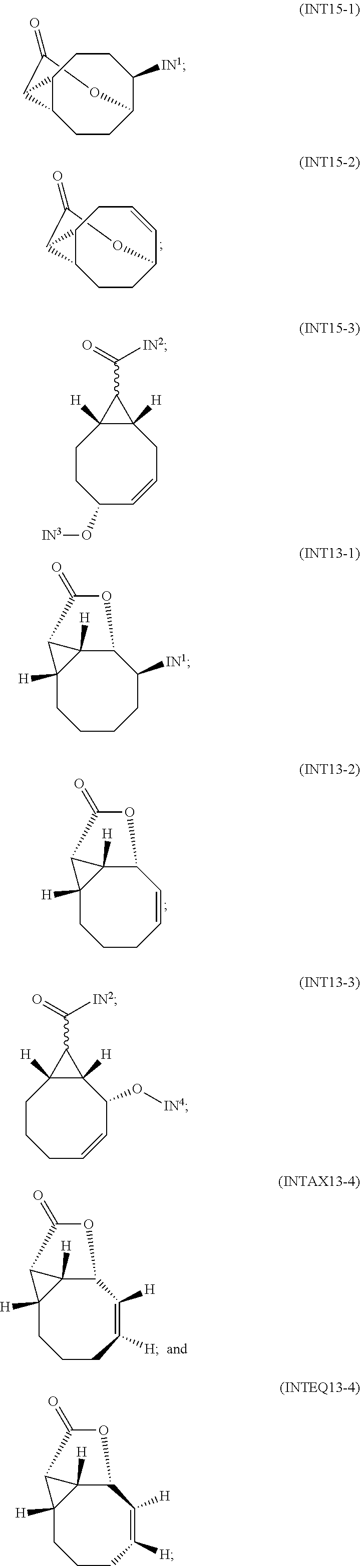
wherein
IN¹ is a halogen;
IN² is —O—C₁₋₄ alkyl or —OH;
IN³ is hydrogen, C₁₋₄ alkyl, or acetyl;
IN⁴ is hydrogen, C₁₋₄ alkyl, or acetyl;
preferably IN¹ is iodine;
preferably IN² is —O—CH₃;
preferably IN³ is hydrogen; and
preferably IN⁴ is acetyl.

16. A method for synthesizing
(i) a compound according to any one of claims 1 to 9, or a salt, solvate, hydrate, and/or an enantiomer thereof, or
(ii) a composition according to claim 10;
wherein said method comprises the step of subjecting a compound Z or a salt, solvate, hydrate, and/or an enantiomer thereof, to photoisomerization, wherein compound Z comprises a (Z)-bicyclo[6.1.0]non-3-ene moiety, wherein at least one allylic carbon of said moiety is in the R-configuration and is substituted with R₄₈;
R₄₈ is selected from the group consisting of —OH, —O-acetyl, —O—C₁₋₄ alkyl, halogen, active carbonate, and a releasable group;
the carbon atom at position 1 of said moiety is in the R-configuration;
the carbon atom at position 8 of said moiety is in the S-configuration; and
preferably the carbon atom at position 9 of said moiety is substituted; and
preferably compound Z is an intermediate, or a salt, solvate, hydrate, and/or an enantiomer thereof, wherein the intermediate is according to Formula (INT15-3) or (INT13-3) as defined in claim 15.


SEQUENCE LISTING (1 sequence)

SEQ ID NO: 1 - protein, 234 aa, synthetic construct
SVQLQQSDAELVKPGASVKISCKASGYTFTDHAIHWVKQNPEQGLEWIGYFSPGNDDFKYNERFKGKATLTADKSSSTAYLQLNSLTSEDSAVYFCTRSLNMAYWGQGTSVTVSSGGGGSDIVMTQSCSSCPVSVGEKVTLSCKSSQSLLYSGNQKNYLAWYQQKPGQSPKLLIYWASTRESGVPDRFTGSGSGTDFTLSISSVETEDLAVYYCQQYYSYPLTFGAGTKLVLKR